(19) United States
(12) Patent Application Publication
(10) Pub. No.: US 2025/0261974 A1
(43) Pub. Date: Aug. 21, 2025

(54) PEDICLE SCREW REDUCER INSTRUMENTS AND METHODS OF USE

(71) Applicant: DePuy Synthes Products, Inc., Raynham, MA (US)

(72) Inventors: Justin Fisk, Franklin, MA (US); Thomas Runco, Providence, RI (US)

(21) Appl. No.: 19/054,514

(22) Filed: Feb. 14, 2025

Related U.S. Application Data
(60) Provisional application No. 63/556,378, filed on Feb. 21, 2024.

(51) Int. Cl. A61B 17/70

(52) U.S. Cl. CPC A61B 17/7086; A61B 17/7091

(57) ABSTRACT

Surgical instruments and methods to aid in reducing or urging a spinal fixation element into a pedicle screw or other implant are disclosed. Disclosed instruments and methods facilitate delivery of a setscrew or other closure, reduction of a rod or other spinal fixation element into a pedicle screw, and securing the rod relative to the pedicle screw using the setscrew. Example instruments and methods can utilize a reducer clip that couples to a proximal end of a pedicle screw receiver head or extension tabs thereof, an inserter-reducer shaft that can couple with a setscrew at its distal end, a reducer handle that can threadably engage with the reducer clip to translate the shaft and thereby reduce a rod, and a driver handle that can rotate the inserter-reducer shaft to engage the setscrew with threads formed on the pedicle screw or extension tabs thereof.

### CROSS-REFERENCE TO RELATED APPLICATIONS

This application claims the benefit of U.S. Provisional Application No. 63/556,378, entitled “Pedicle Screw Reducer Instruments and Methods of Use,” filed on Feb. 21, 2024. The entire contents of this application are incorporated by reference herein.

### FIELD

This disclosure relates generally to surgical instruments and methods of use and, more particularly, to surgical instruments that aid in reducing or urging a spinal fixation element into a pedicle screw or other implant.

### BACKGROUND

Fixation systems can be used in orthopedic surgery or neurosurgery to maintain a desired spatial relationship between multiple bones or bone fragments. For example, in spinal surgery, a spinal fixation system can be implanted into a patient to align and/or fix a desired orientation of one or more vertebrae. A typical spinal fixation system can include bone anchors, such as pedicle screws, implanted in the vertebrae and longitudinal rods or other spinal fixation elements that are secured to the bone anchors by setscrews or other closure mechanisms. Implanting the fixation system can involve multiple steps, e.g., rod reduction, derotation, and setscrew insertion, among others.

One common implantation procedure is rod reduction, whereby a rod or other spinal fixation element is aligned relative to opposed arms of a pedicle screw receiver member and reduced or urged distally toward the pedicle screw into a position where a closure mechanism like a setscrew can be utilized to secure the two components relative to one another. In some instances, a pedicle screw receiver member can have extended tabs that can facilitate alignment of the rod and reduction toward a rod seat at a base of the receiver member. The extended tabs can include threads formed along a portion thereof that can be utilized in connection with a setscrew to enable a greater amount of reduction. In some cases, the tabs can be detached or broken off after the rod is fully reduced and secured using the setscrew.

In certain cases, however, the additional amount of reduction capacity provided by the extended tabs of a pedicle screw receiver member can be insufficient for a given surgical procedure. By way of example, in some cases involving larger patients and minimally invasive surgical techniques, there can be a greater amount of tissue over a vertebra where a pedicle screw is implanted, such that the extended tabs may be insufficient to reach above the tissue and/or a skin surface of the patient. In such cases, there can be a need for additional reduction capacity to complete a procedure.

In addition, certain prior setscrew insertion instruments rigidly position a setscrew or other closure at a distal end of the instrument and, as a result, it can be difficult to properly thread the setscrew into engagement with the threads of the pedicle screw, e.g., on the extension tabs. For example, axial misalignment of the setscrew relative to the pedicle screw receiver member can make engaging the threaded interface difficult and/or damage components, such as the threads of the setscrew.

Accordingly, there is a need for improved pedicle screw reducer instruments that can provide additional reduction capability and alleviate difficulties in surgical procedures like those described herein.

### SUMMARY

The present disclosure addresses the above-noted deficiencies and generally relates to surgical instruments that aid in reducing or urging a spinal fixation element into a pedicle screw or other implant (e.g., another form of bone anchor, etc.). The present disclosure provides streamlined pedicle screw reduction instruments that can facilitate delivery of a setscrew or other closure, reduction of a rod or other spinal fixation element into a pedicle screw, and securing the rod relative to the pedicle screw using the setscrew. Example instruments and methods according to the present disclosure can utilize a reducer clip that couples to a proximal end of a pedicle screw receiver head or extension tabs thereof, an inserter-reducer shaft that can couple with a setscrew at its distal end, a reducer handle that can threadably engage with the reducer clip to translate the shaft and thereby reduce a rod, and a driver handle that can rotate the inserter-reducer shaft to engage the setscrew with threads formed on the pedicle screw or extension tabs thereof.

An example system can include a reducer clip having a lumen extending from a proximal end to a distal end thereof, a recess formed in a sidewall of the reducer clip along a distal portion thereof, and threads formed in the sidewall of the reducer clip along a proximal portion thereof. The system can further include a shaft disposed through the lumen of the reducer clip. The system can further include a reducer handle having a lumen extending from a proximal end to a distal end thereof and threads formed along an outer distal portion thereof, the reducer handle threads interfacing with the threads formed along the proximal portion of the reducer clip. The system can further include a driver handle coupled to a proximal portion of the shaft extending through the reducer handle.

Any of a variety of alternative or additional features can be included and are considered within the scope of the present disclosure. For example, in some embodiments, the driver handle can extend proximal to the reducer handle.

In certain embodiments, the reducer clip can be a monolithic component. In other embodiments, the reducer clip can be a multi-part component.

In some embodiments, the reducer clip can further include a locking sleeve with a locking tab at a distal end thereof that is configured to translate relative to another portion of the reducer clip. In certain embodiments, the reducer clip can further include a biasing element that urges the locking sleeve distally relative to another portion of the reducer clip.

In certain embodiments, the reducer clip can further include opposed tabs extending radially away from a longitudinal axis of the reducer clip.

In some embodiments, the reducer clip can further include a flat formed on an outer proximal portion thereof.

In certain embodiments, the shaft can further include a distal portion configured to rotationally drive a setscrew, a sleeve disposed around the shaft over at least part of the distal portion thereof, and a biasing element disposed proximal to the sleeve and configured to urge the sleeve distally relative to the shaft.

In some embodiments, the shaft can further include a proximal-facing shoulder. This proximal-facing shoulder can act as a bearing surface for the reducer handle to urge the shaft distally relative to a bone anchor and the reducer clip coupled thereto.

In certain embodiments, the shaft can further include a flat formed along a proximal portion thereof. The flat can be utilized to facilitate coupling of the driver handle to the shaft to allow the driver handle to rotate the shaft.

In some embodiments, the reducer handle can further include a distal-facing shoulder disposed proximal to the threads. The distal-facing shoulder can act as a stop configured to contact a proximal-facing portion of the reducer clip and prevent further distal advancement of the reducer handle relative to the reducer clip and bone anchor coupled thereto.

In certain embodiments, the reducer handle can further include a cylindrical proximal portion defining a proximal-facing recess.

In some embodiments, the reducer handle can further include opposed paddles extending radially away from a longitudinal axis of the reducer handle and defining a gap therebetween at a proximal end of the reducer handle.

In certain embodiments, the reducer clip can include a bone anchor extension having an inner sleeve with opposed distally-extending arms and a threaded proximal portion, as well as an outer sleeve disposed over the inner sleeve and configured to translate relative to the inner sleeve to selectively lock the bone anchor extension to a bone anchor.

In some embodiments, the system can further include a bone anchor, wherein the reducer clip is configured to couple to a proximal portion of the bone anchor.

In certain embodiments, the reducer handle can further include a distal-facing shoulder configured to contact a proximal-facing portion of the reducer clip to prevent further distal advancement of the reducer handle relative to the reducer clip.

An example instrument of the present disclosure is a reducer clip that can include a distal sleeve having a lumen extending from a proximal end to a distal end thereof and a recess formed in a sidewall of the distal sleeve along a distal portion thereof. The reducer clip can further include a proximal sleeve having a lumen extending from a proximal end to a distal end thereof and threads formed in a sidewall of the proximal sleeve along a proximal portion thereof. The reducer clip can further include a locking sleeve having a lumen extending from a proximal end to a distal end thereof and a locking tab at the distal end thereof. Further, a distal portion of the proximal sleeve can be received within a proximal portion of the distal sleeve. Further, a distal portion of the locking sleeve can be received within the proximal portion of the distal sleeve. Still further, the locking sleeve can be axially disposed between the distal sleeve and the proximal sleeve and configured to axially translate relative thereto.

As with the systems described above, the instruments disclosed herein can include any of a variety of additional or alternative features that are considered within the scope of the present disclosure. For example, in some embodiments, the reducer clip can further include a biasing element disposed between the locking sleeve and the proximal sleeve that is configured to distally bias the locking sleeve relative to the proximal sleeve.

In certain embodiments, the distal sleeve can include opposed recesses formed in the sidewall of the distal sleeve along a distal portion thereof and the locking sleeve includes opposed locking tabs at the distal end thereof.

In some embodiments, the locking sleeve can further include opposed tabs extending radially away from a longitudinal axis of the reducer clip.

In certain embodiments, a proximal portion of the locking sleeve can surround a portion of the proximal sleeve.

In some embodiments, the proximal sleeve can further include a flat formed on an outer proximal portion thereof.

In certain embodiments, the distal sleeve can be configured to receive a proximal portion of a bone anchor within the lumen thereof.

Another example instrument of the present disclosure is an inserter-reducer shaft that can include an elongate body having a proximal portion with a flat formed thereon, a distal portion configured to rotationally drive a setscrew, a first, distal-facing shoulder formed proximal to the distal portion, and a second, proximal-facing shoulder formed proximal to the first shoulder and distal to the proximal portion. The inserter-reducer shaft can further include a sleeve disposed around the elongate body distal to the first shoulder. The inserter-reducer shaft can further include a biasing element disposed between the sleeve and the first shoulder and configured to bias the sleeve distally relative to the elongate body.

As with the systems and instruments described above, the instruments disclosed herein can include any of a variety of additional or alternative steps that are considered within the scope of the present disclosure. For example, in some embodiments, the sleeve can be constrained against translating relative to the elongate body beyond a distal end of the portion configured to rotationally drive a setscrew.

In certain embodiments, the biasing element can be a coil spring disposed around the elongate body between a proximal end of the sleeve and the first shoulder.

In some embodiments, a portion of the elongate body extending between the first shoulder and the second shoulder can have an outer diameter that is less than a diameter of one or more of the first shoulder and the second shoulder.

In certain embodiments, a portion of the elongate body extending between the first shoulder and the second shoulder can have an outer diameter equal to the diameter of one or more of the first shoulder and the second shoulder.

In some embodiments, an outer diameter of the first shoulder and an outer diameter of the second shoulder can be equal.

In certain embodiments, the inserter-reducer shaft can further include a spring clip configured to aid retention of a setscrew along the distal portion of the elongate body.

In some embodiments, the inserter-reducer shaft can further include a thrust bearing disposed around the elongate body proximal to the second shoulder.

In certain embodiments, the distal portion of the elongate body can further include an insert received within a distal-facing recess of the elongate body.

An example surgical method according to the present disclosure can include coupling a reducer clip to a proximal end of a bone anchor. The method can further include passing a distal portion of a shaft having a setscrew disposed thereon through a lumen of the reducer clip until a distal threaded portion of a reducer handle disposed around the shaft contacts a proximal threaded portion of the reducer clip. The method can further include rotating the reducer handle to engage the threads of the reducer handle with the threads of the reducer clip and advance the shaft distally. The method can further include rotating a driver handle coupled to a proximal end of the shaft extending through the reducer handle to engage threads on an outer surface of the setscrew with threads formed on an inner surface of the bone anchor.

As with the systems and instruments described above, the methods disclosed herein can include any of a variety of additional or alternative steps that are considered within the scope of the present disclosure. For example, in some embodiments, the method can further include passing a proximal end of the shaft through a lumen of the reducer handle.

In certain embodiments, the method can further include coupling the driver handle to the proximal end of the shaft extending through the reducer handle.

In some embodiments, the method can further include loading the setscrew onto a distal end of the shaft. Loading the setscrew onto the distal end of the shaft can include contacting the setscrew against a distally biased sleeve disposed around the shaft.

In certain embodiments, coupling the reducer clip to the bone anchor can include squeezing opposed arms of the bone anchor radially inward toward one another while passing a distal end of the reducer clip over the proximal end of the bone anchor. Further, coupling the reducer clip to the bone anchor can include releasing the opposed arms of the bone anchor after passing the distal end of the reducer clip over the proximal end of the bone anchor such that opposed portions of the opposed arms of the bone anchor extend into opposed recesses formed in a sidewall of the reducer clip.

In some embodiments, coupling the reducer clip to the bone anchor can include proximally withdrawing a locking sleeve of the reducer clip while passing a distal portion of the reducer clip over the proximal end of the bone anchor and distally advancing the locking sleeve after the distal portion of the reducer clip is positioned over the proximal end of the bone anchor.

In certain embodiments, rotating the reducer handle can include contacting a shoulder formed on the reducer handle against a proximal end of the reducer clip to prevent further distal advancement of the reducer handle relative to the reducer clip.

In some embodiments, the method can further include separating the reducer clip, the shaft, the reducer handle, and the driver handle as a single unit from the bone anchor after rotating the driver handle to engage the threads on the setscrew with the threads on the bone anchor.

Any of the features or variations described herein can be applied to any particular aspect or embodiment of the present disclosure in a number of different combinations. The absence of explicit recitation of any particular combination is due solely to avoiding unnecessary length or repetition.

### BRIEF DESCRIPTION OF THE DRAWINGS

The aspects and embodiments of the present disclosure can be more fully understood from the following detailed description taken in conjunction with the accompanying drawings, in which:

FIG. 1A is a proximal perspective view of one embodiment of an assembly, including a pedicle screw reducer instrument coupled to a pedicle screw, setscrew, and rod, according to one embodiment of the present disclosure;

FIG. 1B is a distal perspective view of the assembly of FIG. 1A;

FIG. 2A is a proximal perspective view of the pedicle screw reducer instrument of FIG. 1A;

FIG. 2B is a distal perspective view of the pedicle screw reducer instrument of FIG. 1A;

FIG. 3 is an exploded view of the pedicle screw reducer instrument of FIG. 1A;

FIG. 4 is an exploded view of the pedicle screw and rod of FIG. 1A;

FIG. 5 is a detail view of a rod receiving head of the pedicle screw of FIG. 1A;

FIG. 6 is a detail view of the setscrew of FIG. 1A;

FIG. 7A is a proximal perspective view of a reducer clip of the pedicle screw reducer instrument of FIG. 1A;

FIG. 7B is a distal perspective view of the reducer clip of FIG. 7A;

FIG. 7C is a side view of the reducer clip of FIG. 7A (an opposing side view can be substantially identical);

FIG. 7D is a top view of the reducer clip of FIG. 7A (an opposing bottom view can be substantially identical);

FIG. 7E is a distal view of the reducer clip of FIG. 7A;

FIG. 7F is a proximal view of the reducer clip of FIG. 7A;

FIG. 8 is an exploded view of the reducer clip of FIG. 7A;

FIG. 9 is a cross-sectional view of the reducer clip of FIG. 7A taken along the lines A-A in FIG. 7A;

FIG. 10 is a cross-sectional view of the reducer clip of FIG. 7A taken along the lines B-B in FIG. 7A;

FIG. 11A is a proximal perspective view of a distal sleeve of the reducer clip of FIG. 7A;

FIG. 11B is a distal perspective view of the distal sleeve of FIG. 11A;

FIG. 11C is a side view of the distal sleeve of FIG. 11A (an opposing side view can be substantially identical);

FIG. 11D is a top view of the distal sleeve of FIG. 11A (an opposing bottom view can be substantially identical);

FIG. 11E is a distal view of the distal sleeve of FIG. 11A;

FIG. 11F is a proximal view of the distal sleeve of FIG. 11A;

FIG. 12A is a proximal perspective view of a locking sleeve of the reducer clip of FIG. 7A;

FIG. 12B is a distal perspective view of the locking sleeve of FIG. 12A;

FIG. 12C is a side view of the locking sleeve of FIG. 12A (an opposing side view can be substantially identical);

FIG. 12D is a top view of the locking sleeve of FIG. 12A (an opposing bottom view can be substantially identical);

FIG. 12E is a distal view of the locking sleeve of FIG. 12A;

FIG. 12F is a proximal view of the locking sleeve of FIG. 12A;

FIG. 13A is a proximal perspective view of a proximal sleeve of the reducer clip of FIG. 7A;

FIG. 13B is a distal perspective view of the proximal sleeve of FIG. 13A;

FIG. 13C is a side view of the proximal sleeve of FIG. 13A (an opposing side view can be substantially identical);

FIG. 13D is a top view of the proximal sleeve of FIG. 13A (an opposing bottom view can be substantially identical);

FIG. 13E is a distal view of the proximal sleeve of FIG. 13A;

FIG. 13F is a proximal view of the proximal sleeve of FIG. 13A;

FIG. 14A is a proximal perspective view of a reducer handle of the pedicle screw reducer instrument of FIG. 1A;

FIG. 14B is a distal perspective view of the reducer handle of FIG. 14A;

FIG. 14C is a side view of the reducer handle of FIG. 14A (an opposing side view can be substantially identical);

FIG. 14D is a distal view of the reducer handle of FIG. 14A;

FIG. 14E is a proximal view of the reducer handle of FIG. 14A;

FIG. 15A is a proximal perspective view of a setscrew driver handle of the pedicle screw reducer instrument of FIG. 1A;

FIG. 15B is a distal perspective view of the setscrew driver handle of FIG. 15A;

FIG. 15C is a side view of the setscrew driver handle of FIG. 15A (an opposing side view can be substantially identical);

FIG. 15D is a distal view of the setscrew driver handle of FIG. 15A;

FIG. 15E is a proximal view of the setscrew driver handle of FIG. 15A;

FIG. 16 is an exploded view of the setscrew driver handle of FIG. 15A;

FIG. 17 is a cross-sectional view of the exploded setscrew driver handle of FIG. 16 taken along the lines A-A in FIG. 16;

FIG. 18 is a cross-sectional view of the setscrew driver handle of FIG. 15A taken along the lines A-A in FIG. 15A;

FIG. 19 is a cross-sectional view of the setscrew driver handle of FIG. 15A taken along the lines B-B in FIG. 15A;

FIG. 20 is a cross-sectional view of the setscrew driver handle of FIG. 15A taken along the lines C-C in FIG. 15A;

FIG. 21A is a proximal perspective view of an inserter-reducer shaft of the pedicle screw reducer instrument of FIG. 1A;

FIG. 21B is a distal perspective view of the inserter-reducer shaft of FIG. 21A;

FIG. 21C is a side view of the inserter-reducer shaft of FIG. 21A (an opposing side view can be substantially identical);

FIG. 21D is a top view of the inserter-reducer shaft of FIG. 21A (an opposing bottom view can be substantially identical);

FIG. 21E is a distal view of the inserter-reducer shaft of FIG. 21A;

FIG. 21F is a proximal view of the inserter-reducer shaft of FIG. 21A;

FIG. 22 is an exploded view of the inserter-reducer shaft of FIG. 21A;

FIG. 23 is a detail exploded view of a distal portion of the inserter-reducer shaft of FIG. 21A;

FIG. 24 is a cross-sectional view of the inserter-reducer shaft of FIG. 21A taken along the lines A-A in FIG. 21A;

FIG. 25 is a detail cross-sectional view of a distal portion of the inserter-reducer shaft of FIG. 21A taken along the lines A-A in FIG. 21A;

FIG. 26 is a perspective cross-sectional view of a distal portion of the inserter-reducer shaft of FIG. 21A taken along the lines A-A of FIG. 21A;

FIG. 27 is a cross-sectional view of the inserter-reducer shaft of FIG. 21A taken along the lines B-B in FIG. 21A;

FIG. 28 is a detail cross-sectional view of a distal portion of the inserter-reducer shaft of FIG. 21A taken along the lines H-H in FIG. 21A;

FIG. 29 is a perspective cross-sectional view of a distal portion of the inserter-reducer shaft of FIG. 21A taken along the lines B-B of FIG. 21A;

FIG. 30 is a perspective view of a thrust washer of the inserter-reducer shaft of FIG. 21A;

FIG. 31A is a first perspective view of a distal portion of an elongate body of the inserter-reducer shaft of FIG. 21A;

FIG. 31B is a second perspective view of the distal portion of FIG. 31A;

FIG. 31C is a side view of the distal portion of FIG. 31A (an opposing side view can be substantially identical);

FIG. 31D is a top view of the distal portion of FIG. 31A (an opposing bottom view can be substantially identical);

FIG. 32A is a proximal perspective view of a retention clip of the inserter-reducer shaft of FIG. 21A;

FIG. 32B is a distal perspective view of the retention clip of FIG. 32A;

FIG. 33A is a proximal perspective view of a distal tip of the inserter-reducer shaft of FIG. 21A;

FIG. 33B is a distal perspective view of the distal tip of FIG. 33A;

FIG. 33C is a side view of the distal tip of FIG. 33A (an opposing side view can be substantially identical);

FIG. 33D is a top view of the distal tip of FIG. 33A (an opposing bottom view can be substantially identical);

FIG. 33E is a distal view of the distal tip of FIG. 33A;

FIG. 33F is a proximal view of the distal tip of FIG. 33A;

FIG. 34A is a proximal perspective view of a biasing sleeve of the inserter-reducer shaft of FIG. 21A;

FIG. 34B is a distal perspective view of the biasing sleeve of FIG. 34A;

FIG. 34C is a side view of the biasing sleeve of FIG. 34A (an opposing side view can be substantially identical);

FIG. 34D is a top view of the biasing sleeve of FIG. 34A (an opposing bottom view can be substantially identical);

FIG. 34E is a distal view of the biasing sleeve of FIG. 34A (an opposing proximal view can be substantially identical);

FIG. 35 is a cross-sectional view of a proximal portion of the assembly of FIG. 1A taken along the lines A-A in FIG. 1A;

FIG. 36 is a perspective cross-sectional view of a proximal portion of the assembly of FIG. 1A taken along the lines A-A in FIG. 1A;

FIG. 37 is a cross-sectional view of a distal portion of the assembly of FIG. 1A taken along the lines A-A in FIG. 1A;

FIG. 38 is a perspective cross-sectional view of a distal portion of the assembly of FIG. 1A taken along the lines A-A in FIG. 1A;

FIG. 39 is a cross-sectional view of a proximal portion of the assembly of FIG. 1A taken along the lines B-B in FIG. 1A;

FIG. 40 is a perspective cross-sectional view of a proximal portion of the assembly of FIG. 1A taken along the lines B-B in FIG. 1A;

FIG. 41 is a cross-sectional view of a distal portion of the assembly of FIG. 1A taken along the lines B-B in FIG. 1A;

FIG. 42 is a perspective cross-sectional view of a distal portion of the assembly of FIG. 1A taken along the lines B-B in FIG. 1A;

FIG. 43 illustrates one step in coupling a reducer clip to a pedicle screw;

FIG. 44 illustrates the reducer clip coupled to the pedicle screw of FIG. 43;

FIG. 45 is a perspective cross-sectional view of the reducer clip and pedicle screw of FIG. 43 taken along the lines A-A in FIG. 43 with a lock sleeve of the reducer clip withdrawn proximally;

FIG. 46 is a perspective cross-sectional view of the reducer clip and pedicle screw of FIG. 44 taken along the lines A-A in FIG. 44;

FIG. 47 illustrates one step in assembling components of a pedicle screw reducer instrument according to the present disclosure;

FIG. 48 illustrates various components of a pedicle screw reducer instrument assembled for use;

FIG. 49 illustrates the insertion of the components of FIG. 48 through the reducer clip and pedicle screw of FIG. 44;

FIG. 50 illustrates a position wherein threads of a reducer handle engage a reducer clip;

FIG. 51 illustrates a position wherein the reducer handle is threaded into the reducer clip to a maximum extent to reduce a rod into a pedicle screw;

FIG. 52 illustrates a position wherein a setscrew driver handle is utilized to drive a setscrew into position and secure a rod relative to a pedicle screw;

FIG. 53 is a cross-sectional view of the position of FIG. 51 taken along the lines A-A in FIG. 51;

FIG. 54 is a cross-sectional view of the position of FIG. 52 taken along the lines A-A in FIG. 52;

FIG. 55 is a cross-sectional view of a distal portion of the assembly of FIG. 1A taken along the lines B-B in FIG. 1A in a first position;

FIG. 56 is a cross-sectional view of a distal portion of the assembly of FIG. 1A taken along the lines B-B in FIG. 1A in a second position;

FIG. 57 is a cross-sectional view of a distal portion of the assembly of FIG. 1A taken along the lines B-B in FIG. 1A in a third position;

FIG. 58A is a proximal perspective view of an assembly, including a pedicle screw reducer instrument coupled to a pedicle screw, setscrew, and rod, according to one embodiment of the present disclosure;

FIG. 58B is a distal perspective view of the assembly of FIG. 58A;

FIG. 59 is an exploded view of the assembly of FIG. 58A;

FIG. 60A is a proximal perspective view of a reducer handle of the pedicle screw reducer instrument of FIG. 58A;

FIG. 60B is a distal perspective view of the reducer handle of FIG. 60A;

FIG. 60C is a distal view of the reducer handle of FIG. 60A;

FIG. 60D is a proximal view of the reducer handle of FIG. 60A;

FIG. 60E is a side view of the reducer handle of FIG. 60A (an opposing side view can be substantially identical);

FIG. 61 is an exploded view of the reducer handle of FIG. 60A;

FIG. 62A is a proximal perspective view of an assembly, including a pedicle screw reducer instrument coupled to a pedicle screw, setscrew, and rod, according to one embodiment of the present disclosure;

FIG. 62B is a distal perspective view of the assembly of FIG. 62A;

FIG. 63 is an exploded view of the assembly of FIG. 62A;

FIG. 64A is a proximal perspective view of a reducer clip of the pedicle screw reducer instrument of FIG. 62A;

FIG. 64B is a distal perspective view of the reducer clip of FIG. 64A;

FIG. 64C is a side view of the reducer clip of FIG. 64A (an opposing side view can be substantially identical);

FIG. 64D is a top view of the reducer clip of FIG. 64A (an opposing bottom view can be substantially identical);

FIG. 64E is a distal view of the reducer clip of FIG. 64A;

FIG. 64F is a proximal view of the reducer clip of FIG. 64A;

FIG. 65 is a cross-sectional view of the reducer clip of FIG. 64A taken along the lines A-A in FIG. 64A;

FIG. 66 is a cross-sectional view of the reducer clip of FIG. 64A taken along the lines B-B in FIG. 64A;

FIG. 67A is a proximal perspective view of an inserter-reducer shaft of the pedicle screw reducer instrument of FIG. 62A;

FIG. 67B is a distal perspective view of the inserter-reducer shaft of FIG. 67A;

FIG. 67C is a side view of the inserter-reducer shaft of FIG. 67A (an opposing side view can be substantially identical);

FIG. 67D is a top view of the inserter-reducer shaft of FIG. 67A (an opposing bottom view can be substantially identical);

FIG. 67E is a distal view of the inserter-reducer shaft of FIG. 67A;

FIG. 67F is a proximal view of the inserter-reducer shaft of FIG. 67A;

FIG. 68 is an exploded view of the inserter-reducer shaft of FIG. 67A;

FIG. 69A is a proximal perspective view of an elongate body of the inserter-reducer shaft of FIG. 67A;

FIG. 69B is a distal perspective view of the elongate body of FIG. 69A;

FIG. 69C is a side view of the elongate body of FIG. 69A (an opposing side view can be substantially identical);

FIG. 69D is a top view of the elongate body of FIG. 69A (an opposing bottom view can be substantially identical);

FIG. 69E is a distal view of the elongate body of FIG. 69A;

FIG. 69F is a proximal view of the elongate body of FIG. 69A;

FIG. 70 illustrates one step in coupling a reducer clip to a pedicle screw;

FIG. 71 illustrates the reducer clip coupled to the pedicle screw of FIG. 70;

FIG. 72 illustrates one step in assembling components of a pedicle screw reducer instrument according to the present disclosure;

FIG. 73 illustrates various components of a pedicle screw reducer instrument assembled for use;

FIG. 74 illustrates the insertion of the components of FIG. 73 through the reducer clip and pedicle screw of FIG. 71;

FIG. 75 illustrates a position wherein threads of a reducer handle engage a reducer clip;

FIG. 76 illustrates a position wherein the reducer handle is threaded into the reducer clip to a maximum extent to reduce a rod into a pedicle screw;

FIG. 77 illustrates a position wherein a setscrew driver handle is utilized to drive a setscrew into position and secure a rod relative to a pedicle screw;

FIG. 78 is a cross-sectional view of the position of FIG. 76 taken along the lines A-A in FIG. 76;

FIG. 79 is a cross-sectional view of the position of FIG. 77 taken along the lines A-A in FIG. 77;

FIG. 80A is a proximal perspective view of an assembly, including a pedicle screw reducer instrument coupled to a pedicle screw, setscrew, and rod, according to one embodiment of the present disclosure;

FIG. 80B is a distal perspective view of the assembly of FIG. 80A;

FIG. 81 is an exploded view of the assembly of FIG. 80A;

FIG. 82 is an exploded view of a pedicle screw extension reducer clip of the assembly of FIG. 80A;

FIG. 83 is a cross-sectional view of the pedicle screw extension of FIG. 80A taken along the lines A-A in FIG. 80A;

FIG. 84 is a cross-sectional view of the pedicle screw extension of FIG. 80A taken along the lines B-B in FIG. 80A;

FIG. 85 is a perspective view of the pedicle screw of FIG. 80A;

FIG. 86 is an exploded view of the pedicle screw of FIG. 85;

FIG. 87 is a cross-sectional view of the pedicle screw, rod, and pedicle screw extension of FIG. 80A taken along the lines A-A in FIG. 80A;

FIG. 88 illustrates one step in coupling a pedicle screw to a pedicle screw extension;

FIG. 89 illustrates a pedicle screw coupled to a pedicle screw extension;

FIG. 90 illustrates one step in assembling components of a pedicle screw reducer instrument according to the present disclosure;

FIG. 91 illustrates various components of a pedicle screw reducer instrument assembled for use;

FIG. 92 illustrates the insertion of the components of FIG. 91 through the pedicle screw extension of FIG. 89;

FIG. 93 illustrates a position wherein threads of a reducer handle engage a pedicle screw extension;

FIG. 94 illustrates a position wherein the reducer handle is threaded into the pedicle screw extension to a maximum extent to reduce a rod into a pedicle screw;

FIG. 95 is a cross-sectional view of the assembly of FIG. 94 taken along the lines A-A in FIG. 94 with a setscrew in a first position;

FIG. 96 is a cross-sectional view of the assembly of FIG. 94 taken along the lines A-A in FIG. 94 with a setscrew in a second position;

FIG. 97A is a proximal perspective view of an assembly, including an adjacent level reducer instrument coupled to a pedicle screw, according to one embodiment of the present disclosure;

FIG. 97B is a distal perspective view of the assembly of FIG. 97A;

FIG. 98 is an exploded view of the assembly of FIG. 97A;

FIG. 99A is a proximal perspective view of a reducer clip of the adjacent level reducer instrument of FIG. 97A;

FIG. 99B is a distal perspective view of the reducer clip of FIG. 99A;

FIG. 99C is a side view of the reducer clip of FIG. 99A (an opposing side view can be substantially identical);

FIG. 99D is a top view of the reducer clip of FIG. 99A (an opposing bottom view can be substantially identical);

FIG. 99E is a distal view of the reducer clip of FIG. 99A;

FIG. 99F is a proximal view of the reducer clip of FIG. 99A;

FIG. 100 is a cross-sectional view of the reducer clip of FIG. 99A taken along the lines A-A in FIG. 99A;

FIG. 101 is a cross-sectional view of the reducer clip of FIG. 99A taken along the lines B-B in FIG. 99A;

FIG. 102A is a side view of a reducer shaft of the adjacent level reducer instrument of FIG. 99A (an opposing side view can be substantially identical);

FIG. 102B is a top view of the reducer shaft of FIG. 102A (an opposing bottom view can be substantially identical);

FIG. 102C is a distal view of the reducer shaft of FIG. 102A;

FIG. 102D is a proximal view of the reducer shaft of FIG. 102A;

FIG. 103A is a side view of a reducer handle of the adjacent level reducer instrument of FIG. 99A (an opposing side view can be substantially identical);

FIG. 103B is a top view of the reducer handle of FIG. 103A (an opposing bottom view can be substantially identical);

FIG. 103C is a distal view of the reducer handle of FIG. 103A;

FIG. 103D is a proximal view of the reducer handle of FIG. 103A;

FIG. 104 is an exploded view of the reducer handle of FIG. 103A;

FIG. 105 illustrates one step in coupling a reducer clip to a pedicle screw;

FIG. 106 illustrates the reducer clip coupled to the pedicle screw of FIG. 105;

FIG. 107 illustrates one step in assembling components of an adjacent level reducer instrument according to the present disclosure;

FIG. 108 illustrates the insertion of the components of FIG. 107 through the reducer clip and pedicle screw of FIG. 106;

FIG. 109 illustrates a position wherein threads of a reducer shaft engage a reducer clip;

FIG. 110 illustrates a position wherein the reducer shaft is threaded into the reducer clip to a maximum extent to reduce a rod into a pedicle screw;

FIG. 111 is a longitudinal cross-sectional view of a distal portion of one embodiment of an inserter-reducer shaft including an elastomeric setscrew biasing element;

FIG. 112 is a longitudinal cross-sectional view of a distal portion of one embodiment of an inserter-reducer shaft including an internally-disposed biasing element; and

FIG. 113 is a longitudinal cross-sectional view of a distal portion of one embodiment of an inserter-reducer shaft including an elastomeric setscrew retaining element.

### DETAILED DESCRIPTION

Certain example embodiments will now be described to provide an overall understanding of the principles of the structure, function, manufacture, and use of the devices, systems, and methods disclosed herein. One or more examples of these embodiments are illustrated in the accompanying drawings. The devices, systems, and methods specifically described herein and illustrated in the accompanying drawings are non-limiting embodiments. The features illustrated or described in connection with one embodiment may be combined with the features of other embodiments. Such modifications and variations are intended to be included within the scope of the present disclosure.

As noted above, the present disclosure generally relates to surgical instruments that aid in reducing or urging a spinal fixation element toward a pedicle screw or other implant (e.g., another bone anchor, etc.). The present disclosure provides streamlined reduction instruments that can facilitate delivery of a setscrew or other closure or locking element, reduction of a rod or other spinal fixation element into a pedicle screw or other bone anchor or implant, and securing the rod relative to the pedicle screw or other bone anchor or implant using the setscrew or other closure or locking element. Example instruments and methods according to the present disclosure can utilize a reducer clip that couples to a proximal end of a pedicle screw receiver head or extension tabs thereof, an inserter-reducer shaft that can couple with a setscrew at its distal end, a reducer handle that can threadably engage with the reducer clip to translate the shaft and thereby reduce a rod, and a driver handle that can rotate the inserter-reducer shaft to engage the setscrew with threads formed on the pedicle screw or extension tabs thereof.

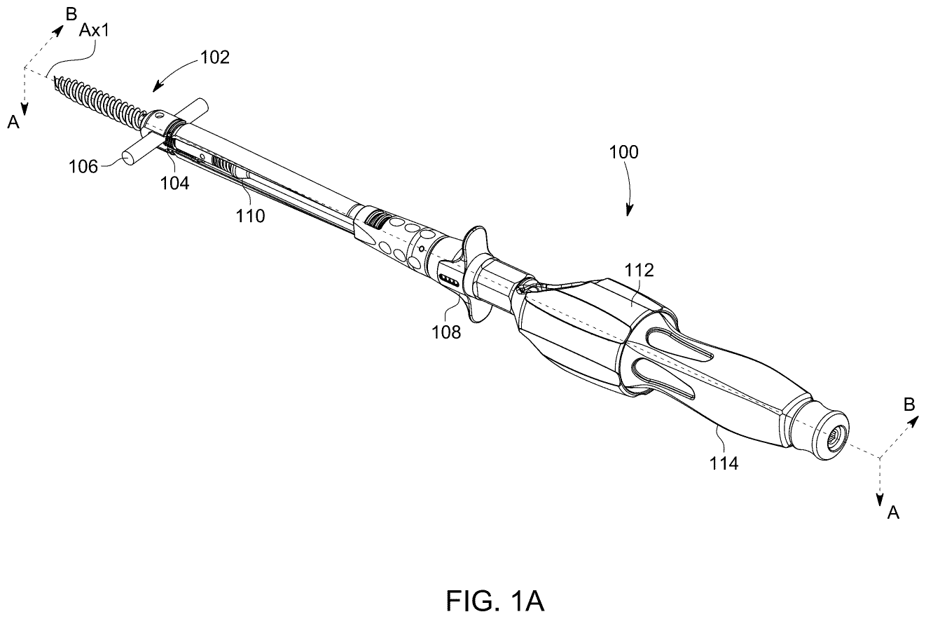

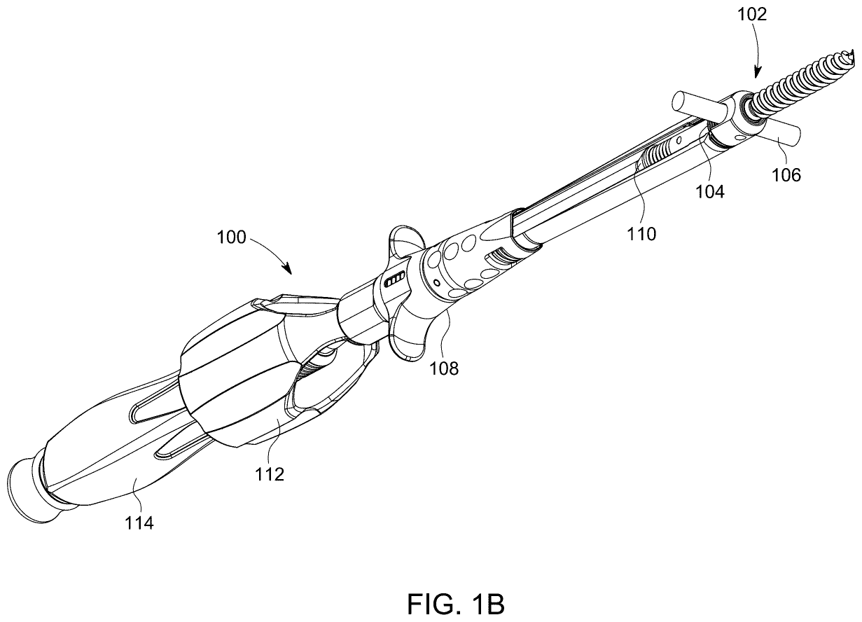

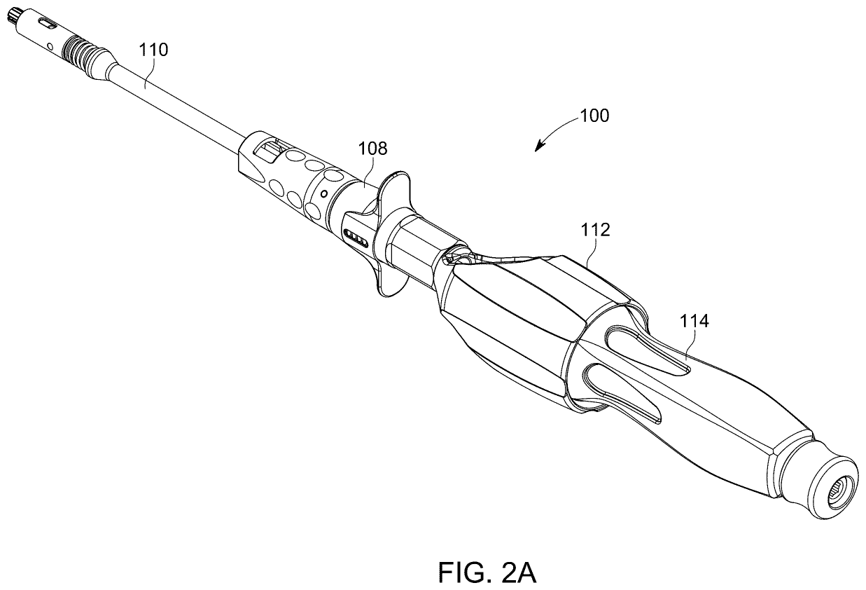

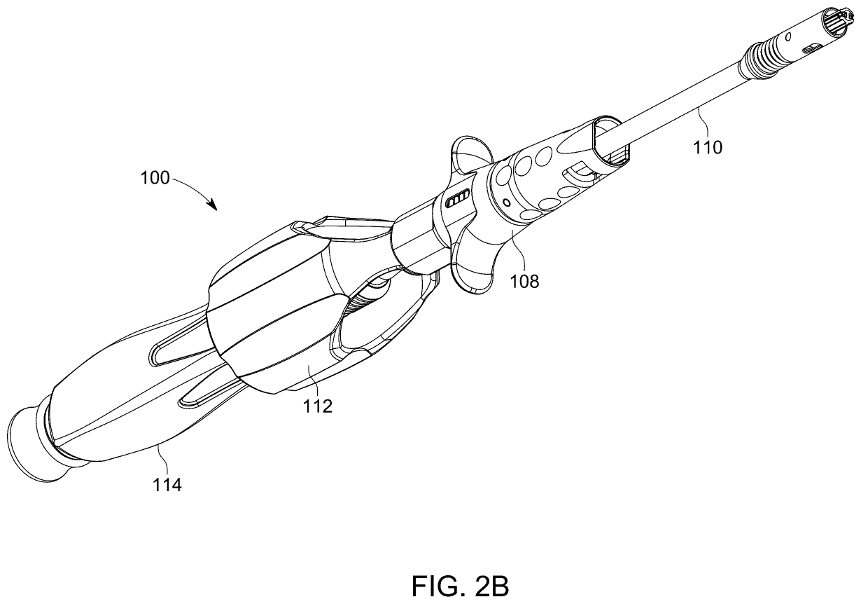

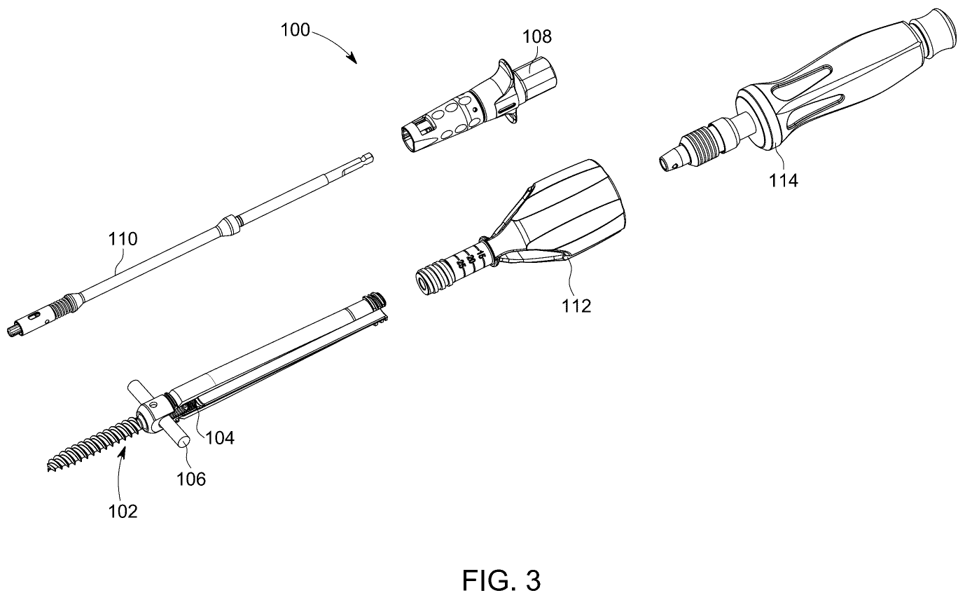

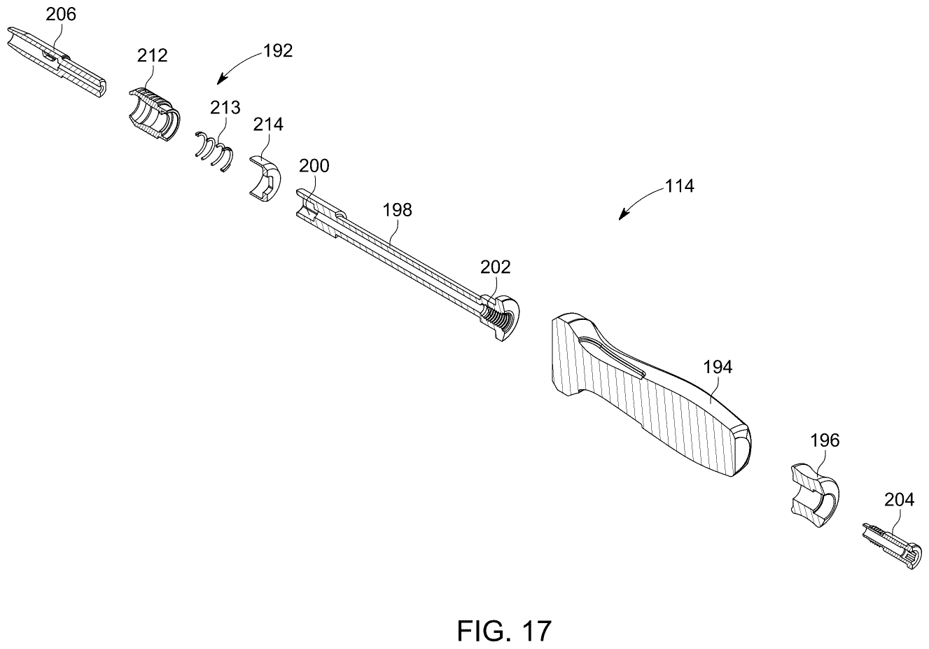

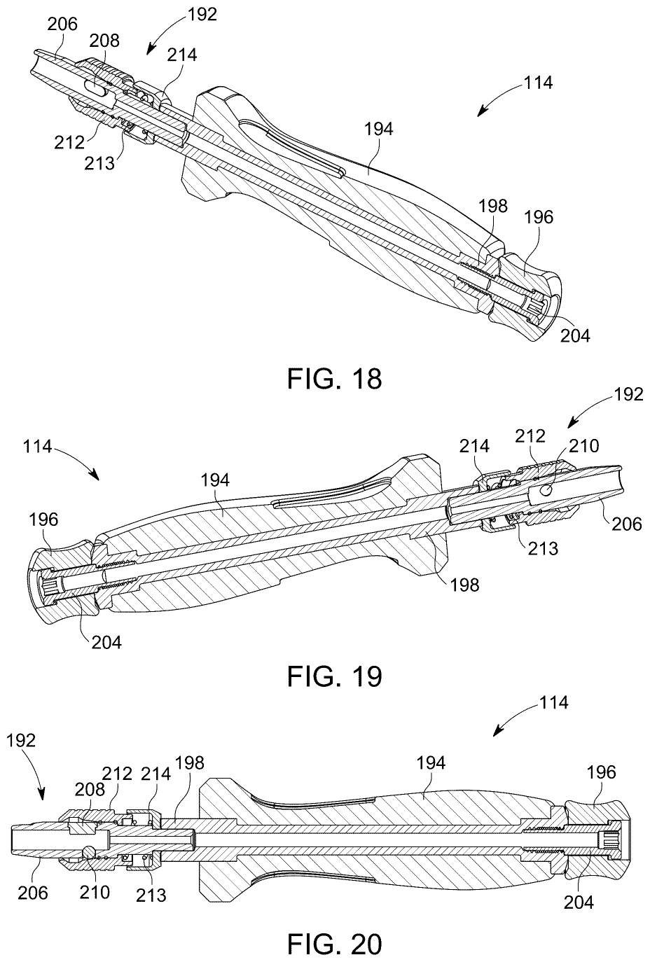

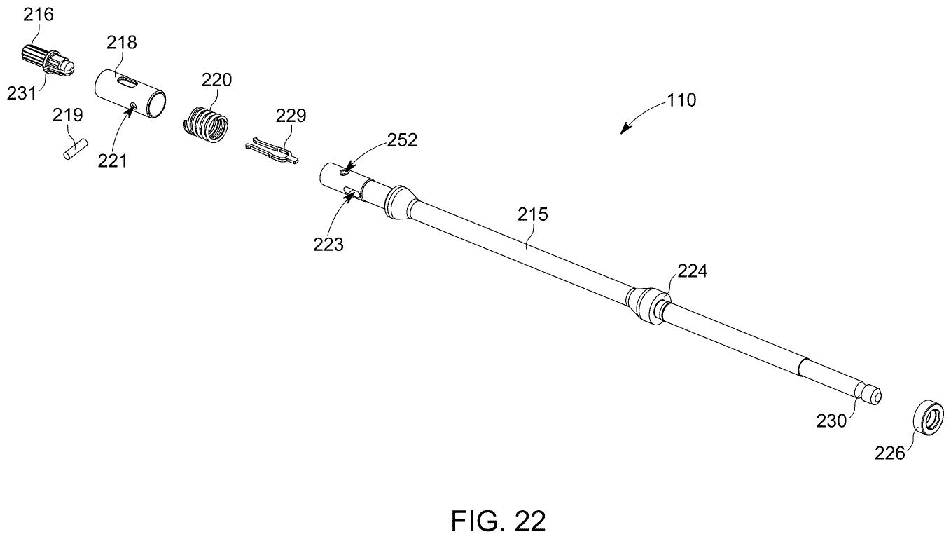

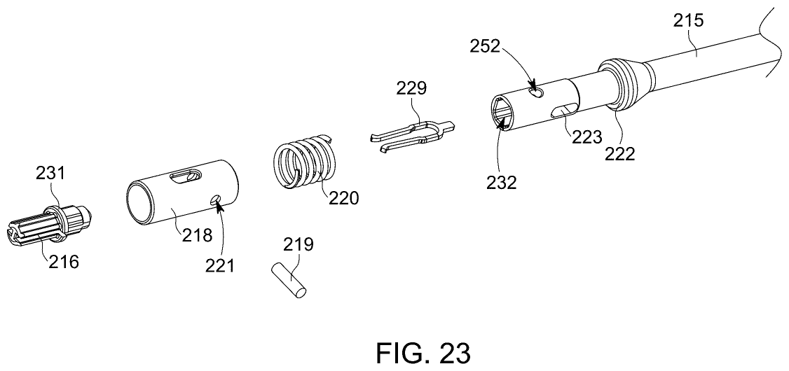

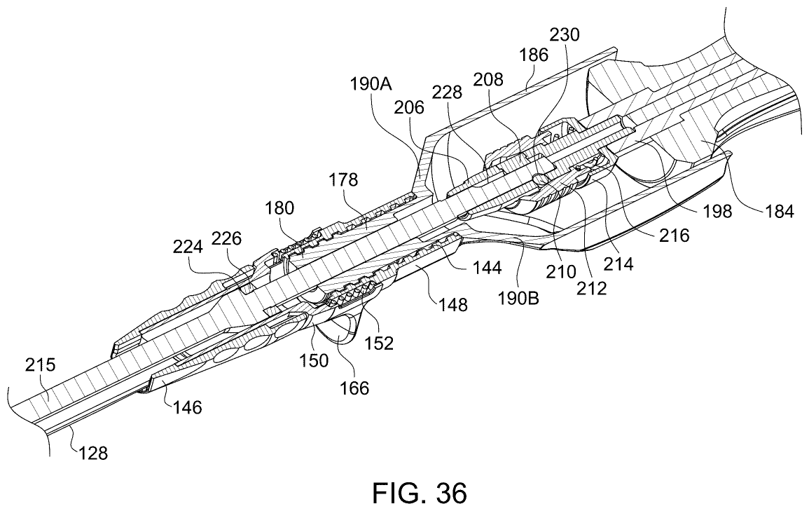

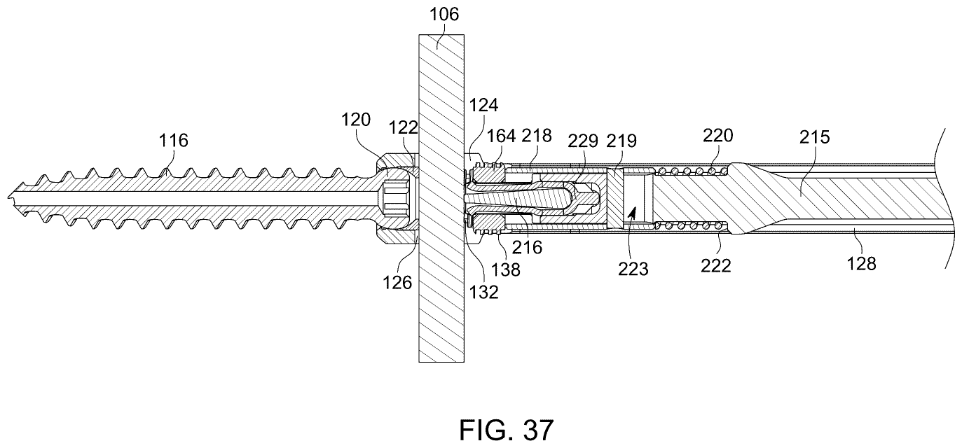

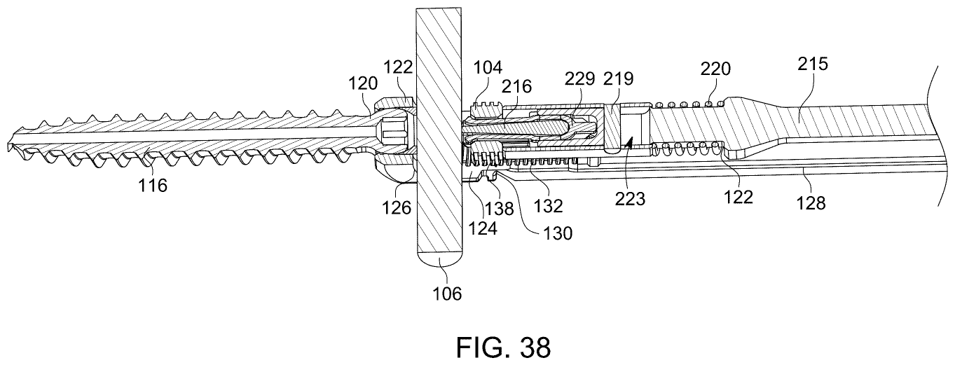

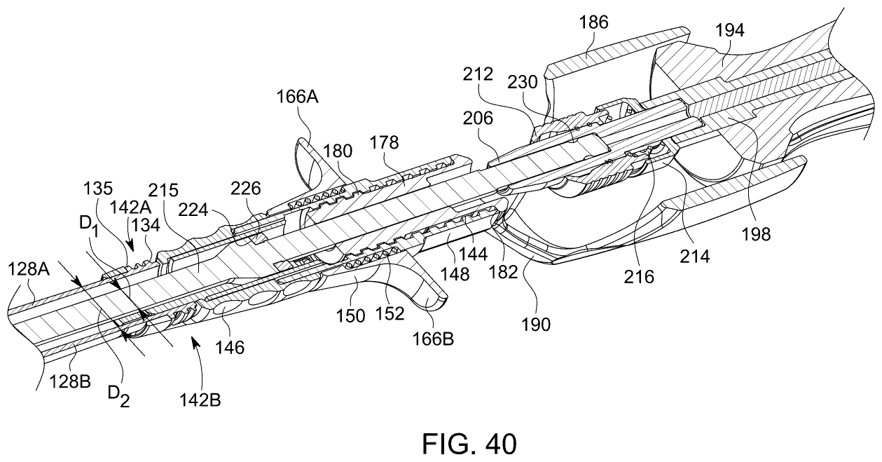

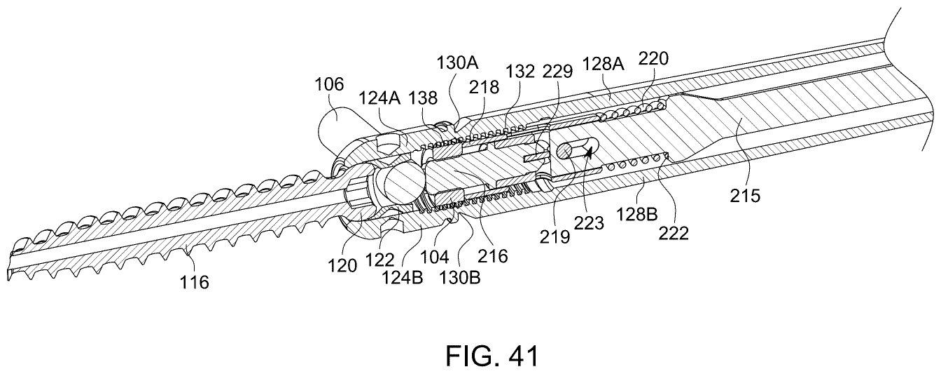

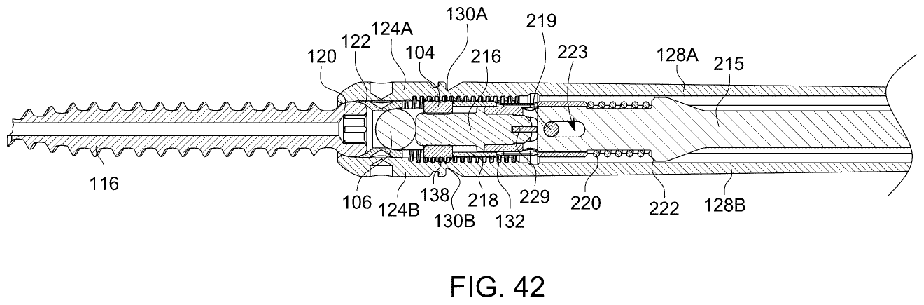

FIGS. 1-57 illustrate a first embodiment of a pedicle screw reducer instrument 100 according to the present disclosure. FIGS. 1A and 1B illustrate an assembly of the instrument 100 coupled to a pedicle screw 102, setscrew 104, and rod 106, according to one embodiment of the present disclosure. The pedicle screw reducer instrument 100 is shown in isolation in FIGS. 2A and 2B, and FIG. 3 shows an exploded view of the components of the assembly of FIG. 1A. The instrument 100 can include a reducer clip 108, an inserter-reducer shaft 110, a reducer handle 112, and a driver handle 114.

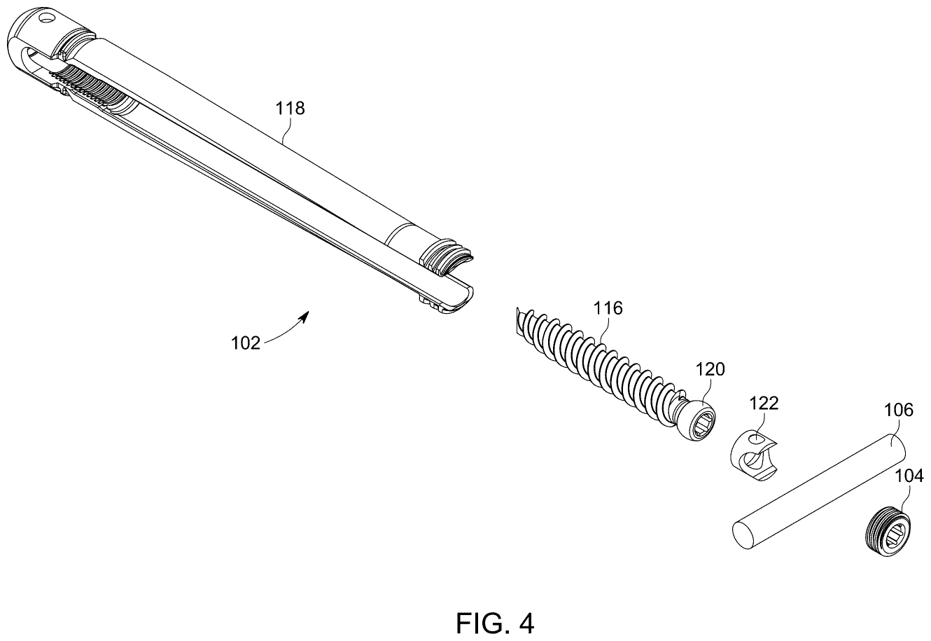

FIGS. 4-6 illustrate an example pedicle screw 102, setscrew 104, and spinal fixation rod 106 in greater detail. The illustrated embodiments are examples only and the scope of the present disclosure contemplates use of the instruments and methods disclosed herein with a variety of implants, including any of a variety of pedicle screws and other bone anchors, as well as any of a variety of spinal fixation elements and locking or closure elements, such as setscrews. As shown in FIG. 4, the pedicle screw 102 can include a shank 116 configured to be driven into bone and a rod receiving head or receiver member 118 configured to receive the spinal fixation rod 106. The receiver member 118 can include a polyaxial seat at a distal end thereof that can be configured to receive a proximal head 120 of the shank 116 such that the receiver member 118 can move polyaxially relative to the shank when coupled thereto. The pedicle screw 102 can also include a compression cap 122 that can be received within the receiver member 118 proximal to the proximal head 120 of the shank 116. The compression cap 122 can include a distal surface configured to contact the proximal head 120 of the shank 116, e.g., a hemispherical recess with a diameter matched to a diameter of proximal head 120 of the shank 116. The compression cap 122 can also include a proximal-facing rod seat configured to receive the rod 106, e.g., opposed arms defining a U-shaped surface configured to seat the rod.

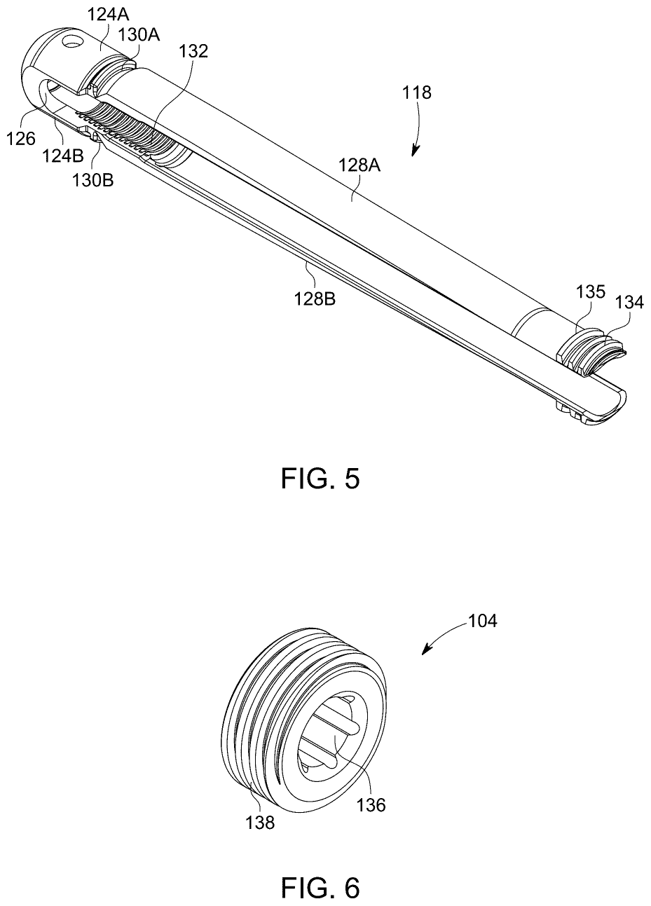

FIG. 5 illustrates the receiver member 118 in greater detail. The receiver member 118 can include opposed arms 124A, 124B that can form a U-shaped recess or rod seat 126 therebetween configured to receive the rod 106. In some embodiments, each of the opposed arms 124A, 124B an include a proximally extending tab 128A, 128B that can aid in capturing and reducing the rod 106 toward the U-shaped rod seat 126. The tabs 128A, 128B can be coupled to the arms 124A, 124B by a reduced-thickness portion of material 130A, 130B. That is, in some embodiments the arms 124A, 124B and tabs 128A, 128B are formed of a single piece of material with a reduced-thickness connecting portion 130A, 130B. The reduced-thickness material 130A, 130B can provide some flexibility to accommodate radial inward/outward deflection or movement of the tabs in response to force applied thereto (as discussed in more detail below), and can serve as a break point facilitating separation of the extended tabs 128A, 128B from the receiver member 118 after the rod 106 is captured and secured to the receiver member using the setscrew 104. An example pedicle screw with similar features is the VIPER® Extended Tab Screw made by DePuy Synthes of Raynham, Massachusetts.

An inner surface of each arm 124A, 124B can include threads 132 formed thereon. The threads 132 can extend onto the inner surfaces of the proximally extending tabs 128A, 128B for some distance. In some embodiments, the threads 132 can extend along about 10 mm of the tabs 128A, 128B, thereby providing an additional about 10 mm of possible reduction beyond that provided by the arms 124A, 124B when using the setscrew 104 to reduce the rod 106 toward the seat 126 of the receiver member 118. A proximal portion of each extended tab 128A, 128B can include one or more surface features formed on an outer surface thereof. For example, each extended tab 128A, 128B can include threads 134 and/or a flange 135 formed on an external surface thereof along a distal portion thereof. The threads 134 and/or flange 135 can be utilized, for example, to facilitate coupling the pedicle screw 102 with other components, as discussed in more detail below.

FIG. 6 illustrates the setscrew 104 in greater detail. The setscrew 104 includes a drive feature 136 formed in a center portion thereof. The drive feature 136 can be a recess or through-hole including one or more substantially flat surfaces to facilitate imparting torque to the setscrew using a driver inserted into the drive feature. In the illustrated embodiment, the drive feature 136 is a through-hole extending through a full thickness of the setscrew 104 and includes a hexagonal arrangement of substantially flat surfaces disposed around a circumference of the through-hole with curved recesses disposed between adjacent flat surfaces. The setscrew 104 also includes threads 138 formed on an outer surface thereof. The threads 138 can be configured to interface with the threads 132 formed on the inner surfaces of the receiver member 118. An example setscrew with similar features is the VIPER® 2 setscrew with X25 drive feature made by DePuy Synthes of Raynham, Massachusetts.

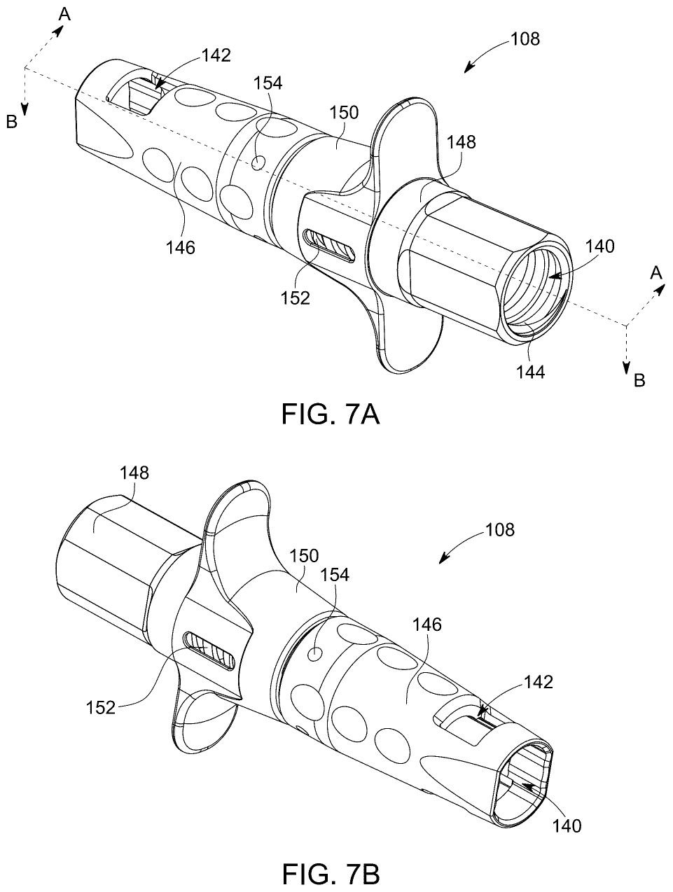

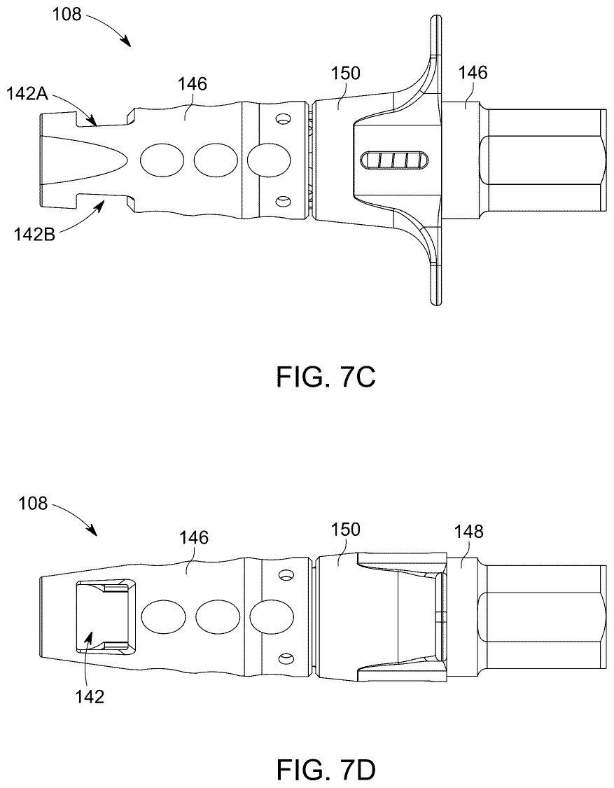

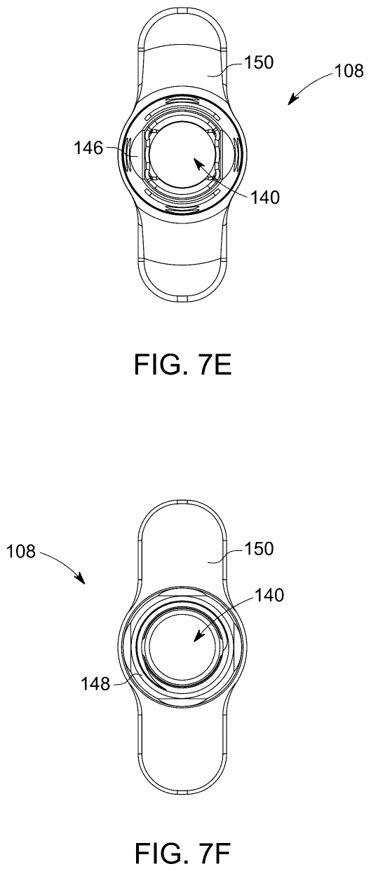

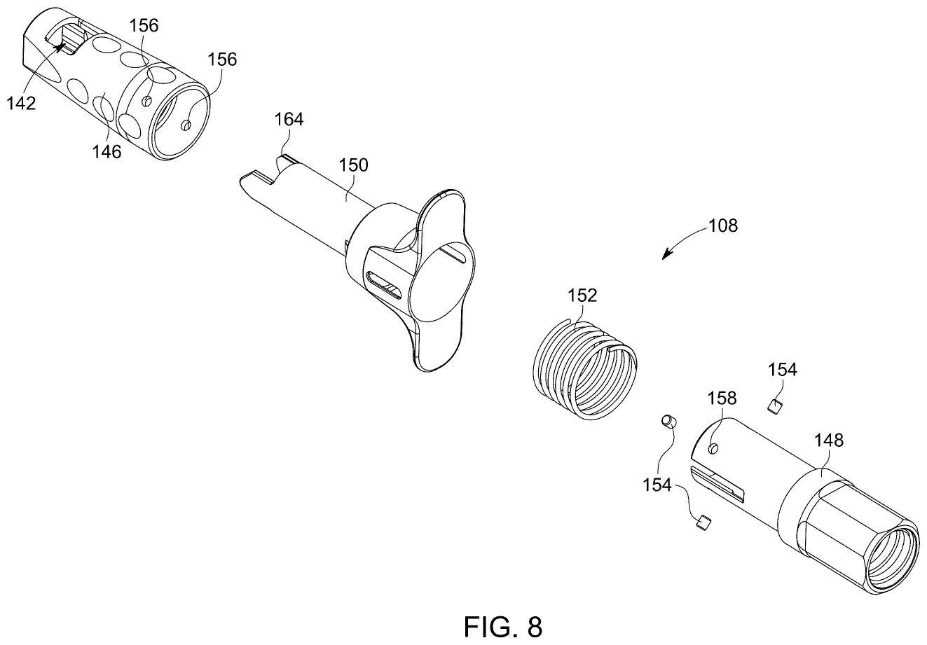

FIGS. 7A-10 illustrate the reducer clip 108 of the pedicle screw reducer instrument 100. The reducer clip 108 can include a lumen 140 extending from a proximal end to a distal end thereof, a recess 142 formed in a sidewall of the reducer clip along a distal portion thereof, and threads 144 formed in the sidewall of the reducer clip along a proximal portion thereof (in other embodiments, the threads 144 can be formed along a more distal portion of the reducer clip, such as a central portion or distal portion thereof). In the illustrated embodiment, and as shown particularly in the exploded and cross-sectional views of FIGS. 8-10, the reducer clip 108 can include a distal sleeve 146 having a lumen extending from a proximal end to a distal end thereof and the recess 142 formed in a sidewall of the distal sleeve along a distal portion thereof. The reducer clip 108 can also include a proximal sleeve 148 having a lumen extending from a proximal end to a distal end thereof and the threads 144 formed in a sidewall of the proximal sleeve along a proximal portion thereof. The reducer clip 108 can further include a locking sleeve 150 having a lumen extending from a proximal end to a distal end thereof and one or more locking tabs 164 at a distal end thereof.

The distal sleeve 146, proximal sleeve 148, and locking sleeve 150 can be assembled such that a distal portion of the proximal sleeve 148 is received within a proximal portion of the distal sleeve 146. Further, a distal portion of the locking sleeve 150 can be received within the proximal portion of the distal sleeve 146. Moreover, a proximal portion of the locking sleeve 150 can surround a portion of the proximal sleeve 148. Still further, the locking sleeve 150 can be axially disposed (e.g., along the longitudinal axis Ax1 of the instrument 100 shown in FIG. 1A) between the distal sleeve 146 and the proximal sleeve 148 and configured to axially translate (e.g., along the longitudinal axis Ax1) relative thereto.

The reducer clip 108 can also include a biasing element 152 disposed between the locking sleeve 150 and the proximal sleeve 148 that is configured to distally bias the locking sleeve relative to the proximal sleeve. In the illustrated embodiment, the biasing element 152 is a coil spring disposed around the proximal sleeve 148. In other embodiments, other known forms of biasing elements can be employed. Further, the reducer clip 108 can include one or more pins 154 that can extend through one or more bores 156 formed in the distal sleeve 146 and one or more bores 158 formed in the proximal sleeve 148 in order to couple the two sleeves such that they do not move relative to one another and to trap the locking sleeve 150 and biasing element 152 axially therebetween. In alternative embodiments, a number of other mechanisms can be utilized to couple the distal sleeve 146 and the proximal sleeve 148. For example, the two sleeves can be welded together without the use of pins 154. In other examples, the two sleeves could threadably couple together and could, in some embodiments, utilize a thread-locking adhesive to prevent separation thereof. In still other examples, any of a variety of fastening techniques can be utilized, including any of a variety of adhesives, mechanical fasteners, interference fits, etc.

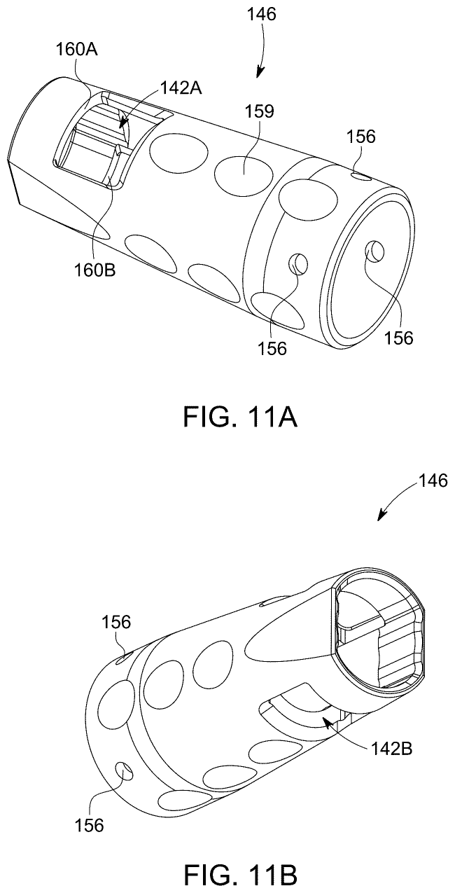

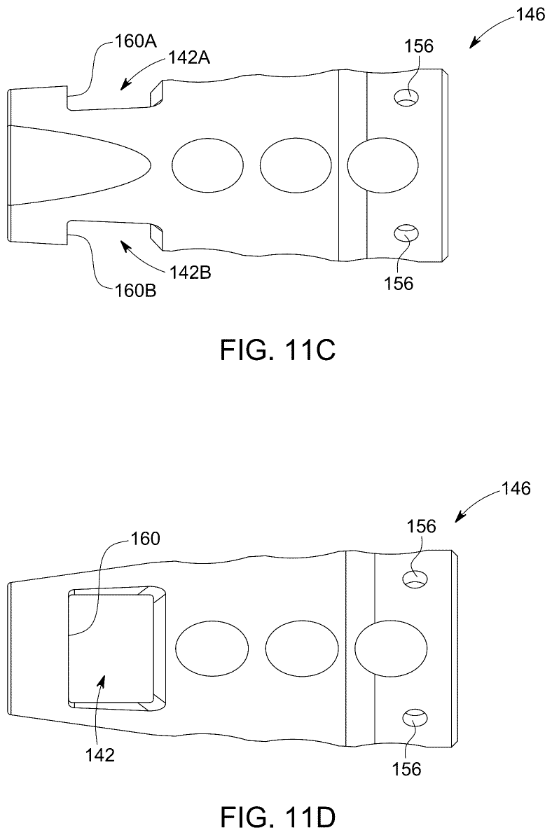

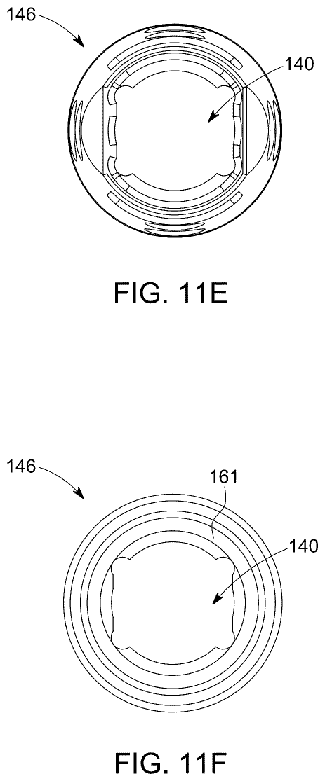

FIGS. 11A-11F illustrate the distal sleeve 146 in isolation. The distal sleeve 146 can have a tapered, generally cylindrical or conical form with a lumen extending therethrough along a longitudinal axis thereof. The distal sleeve can include one or more features, such as recesses 159, formed on an external surface thereof to facilitate a user grasping and/or gripping the component. The lumen extending through the distal sleeve 146 can include a step or shoulder 161 formed along a sidewall thereof to accommodate a distal portion of the proximal sleeve 148 being received therein.

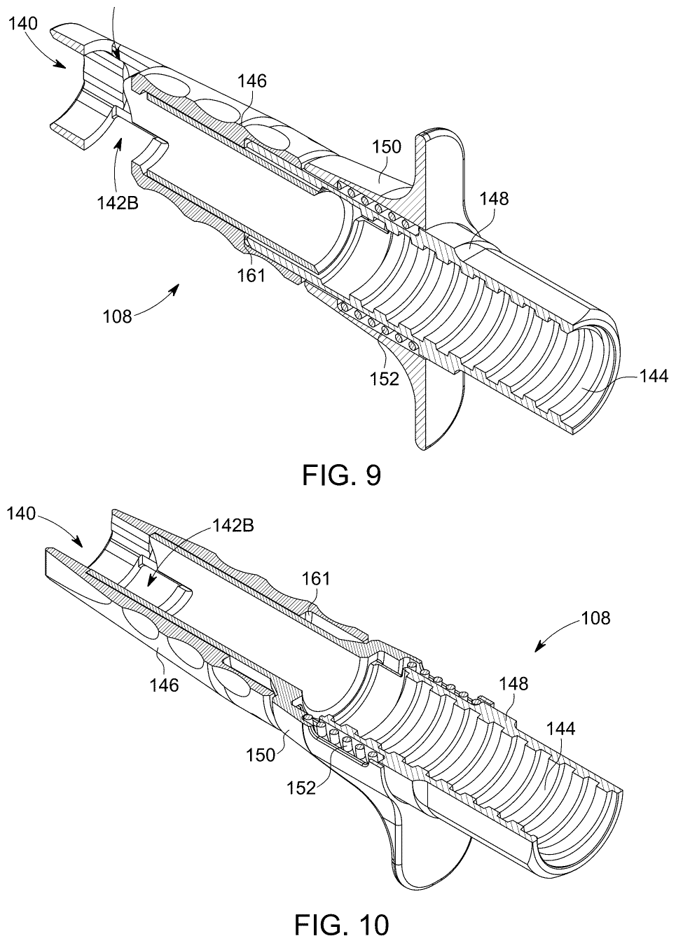

As noted above, the reducer clip 108 can include a recess 142 formed in a sidewall thereof. In the illustrated embodiment, the distal sleeve 146 of the reducer clip 108 includes opposed recesses 142A, 142B (as shown in FIGS. 7C and 9) formed along a distal portion thereof that communicate with the lumen 140 extending through the reducer clip. As shown in FIGS. 7C and 7D, the recesses 142A, 142B can be aligned such that they form a through-bore extending in a direction that is perpendicular to the longitudinal axis Ax1. The recesses 142A, 142B can have a shape configured to receive a portion of the pedicle screw 102, e.g., the external threads 134 and flange 135 formed on a proximal portion of the extended tabs 128A, 128B of the receiver member 118. For example, the recesses 142A, 142B can each have a generally rectangular shape and define distal surfaces 160A, 160B (see FIGS. 11A-11D) configured to contact the flange 135 formed distal to the external threads 134 to prevent proximal movement of the reducer clip 108 relative to the pedicle screw 102 when the threads 134 and/or flange 135 on each of the extended tabs 128A, 128B are disposed within the recesses 142A, 142B. The recesses 142A, 142B can also include counterpart proximal surfaces 162A, 162B configured to serve a similar purpose in contacting a proximal end of the threads 134 and/or the extended tabs 128 to prevent distal movement of the reducer clip 108 relative to the pedicle screw 102 when the threads 134 and/or extended tabs 128 are disposed within the recesses 142.

While the distal sleeve 146 of the reducer clip 108 described above is configured to couple to a pedicle screw or other bone anchor as detailed, other embodiments are within the scope of the present disclosure. For example, in one embodiment the reducer clip 108, and distal sleeve 146 in particular, can be configured to threadably couple with a proximal portion of a bone anchor. For example, a distal portion of the inner lumen of the distal sleeve 146 can include threads configured to interface with the threads 134 formed on the extended tabs 128A, 128B, thereby facilitating threaded coupling between the reducer clip and bone anchor.

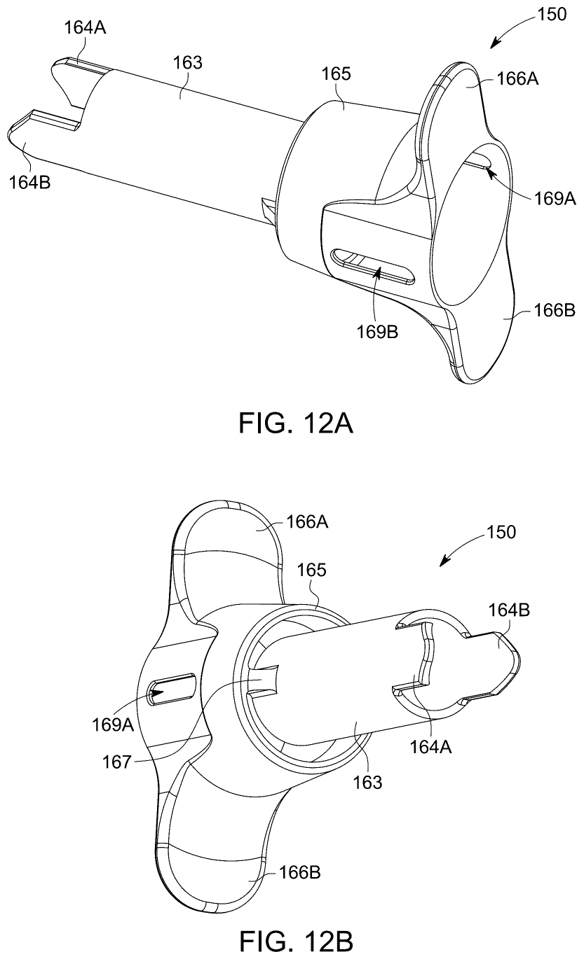

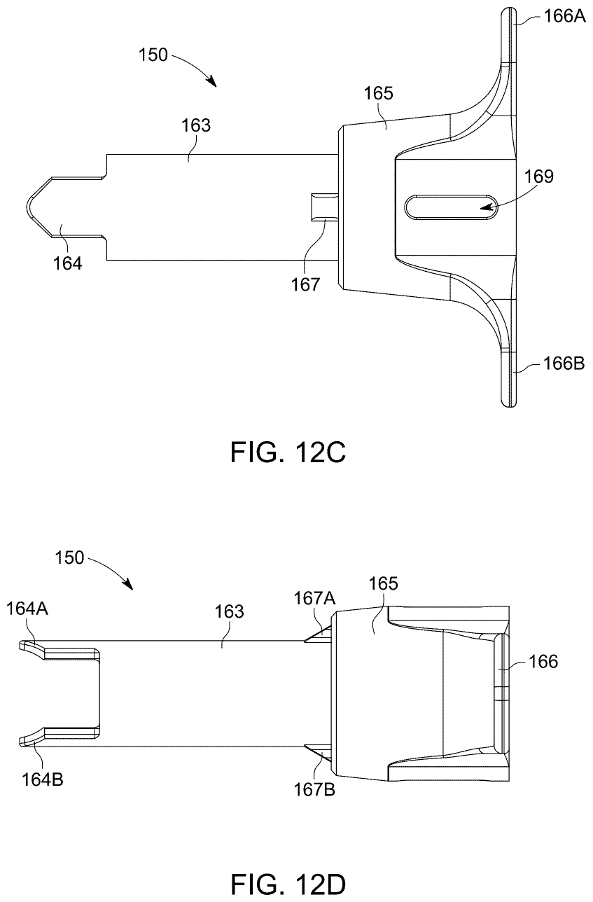

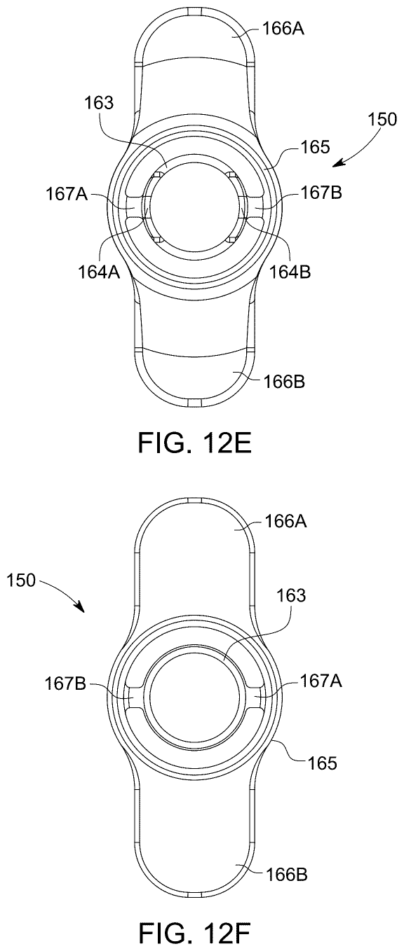

FIGS. 12A-12F illustrate the locking sleeve 150 in isolation. The locking sleeve 150 can include a distal cylindrical body or portion 163 having a diameter configured to be received within the lumen of the distal sleeve 146 and the proximal sleeve 148. The locking sleeve 150 can also include a proximal, generally cylindrical body or portion 165 having a larger diameter than the distal cylindrical body 163. The proximal body 165 can be configured such that a lumen thereof is large enough to surround a proximal portion of the distal cylindrical body 163, as well as the distal sleeve 146 and the proximal sleeve 148. The distal body 163 and the proximal body 165 can be coupled by one or more spans 167 extending therebetween. In the illustrated embodiment, two opposed spans 167A, 167B are shown. Further, the locking sleeve can include one or more openings formed therein to facilitate visualization of other components of the reducer clip 108 and/or cleaning of the component. For example, the locking sleeve can include one or more elongate openings 169 that can facilitate visualization of the biasing spring 152 and/or cleaning of same. In the illustrated embodiment, the locking sleeve 150 includes two opposed openings 169A, 169B. The elongate openings 169 can also provide guidance to a user to prevent them from grasping the area while installing the clip (e.g., grasping the reducer clip 108 by the locking sleeve 150 alone would make coupling with a pedicle screw 102 difficult, as the locking sleeve 150 needs to translate relative to the remainder of the reducer clip and pedicle screw during coupling and separation).

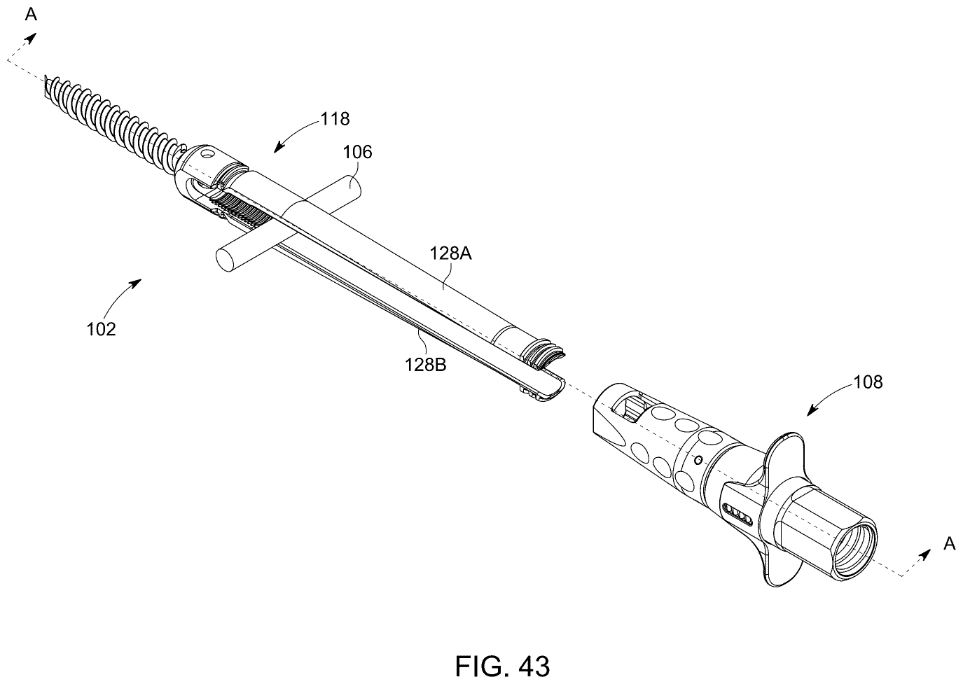

The locking sleeve 150 can include one or more locking tabs 164 at a distal end thereof, e.g., extending from the distal cylindrical body 163. As shown in FIGS. 12A-12F, the locking sleeve 150 can include two opposed locking tabs 164A, 164B. The locking tabs 164 can be protrusions along a distal portion of the locking sleeve 150 configured to advance into a space between the opposed extended tabs 128A, 128B of the pedicle screw 102 and prevent radially inward movement of the tabs toward one another, which would result in movement of the tabs out of the recesses 142A, 142B and would allow axial movement of the reducer clip 108 relative to the pedicle screw. Accordingly, axial movement of the locking sleeve 150 relative to the remainder of the reducer clip 108 can selectively lock the reducer clip to a pedicle screw, as explained in more detail below with regard to FIGS. 43-46.

The locking sleeve 150 can include one or more features to facilitate a user grasping and/or gripping and/or interacting therewith. For example, the locking sleeve 150 can include tabs 166 extending radially away from the longitudinal axis Ax1 of the instrument 100 (shown in FIG. 1A). In the illustrated embodiment, for example, the locking sleeve 150 includes two opposed tabs 166A, 166B extending from a proximal portion of the proximal body 165. The opposed tabs 166A, 166B can provide a syringe-like grip to a user when coupling the reducer clip 108 to a pedicle screw 102. For example, a user can place a finger distal to each opposed tab 166A, 166B with a thumb or palm braced against a proximal end surface 177 of the reducer clip 108. The user can then use their fingers to draw the locking sleeve 150 proximally relative to the remainder of the reducer clip 108 against the biasing force of the spring 152. As explained in more detail below, proximally withdrawing the locking sleeve 150 can allow coupling of the reducer clip 108 to a pedicle screw 102, and distal advancement of the locking sleeve 150 can selectively lock the reducer clip 108 and pedicle screw 102 against separation therebetween by positioning the locking tabs 164A, 164B in a position where they interfere with radially-inward movement of proximal ends of the extended tabs 128A, 128B of the pedicle screw 102.

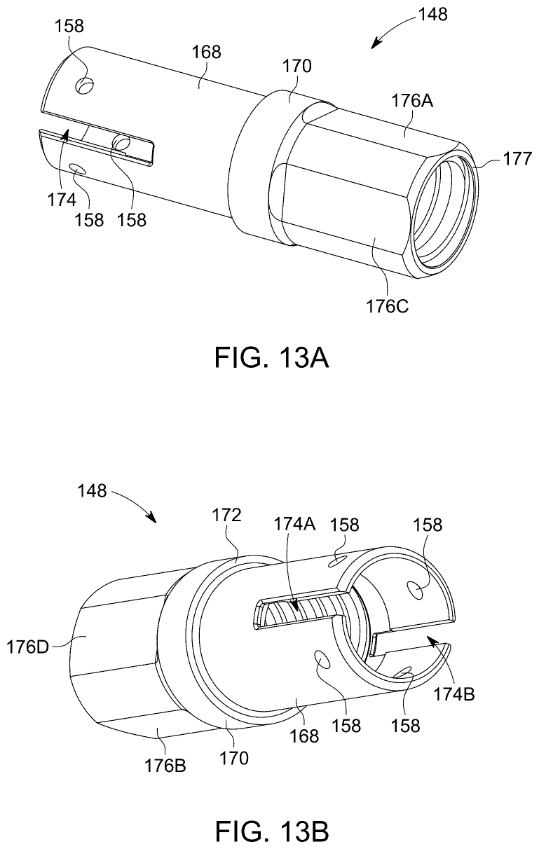

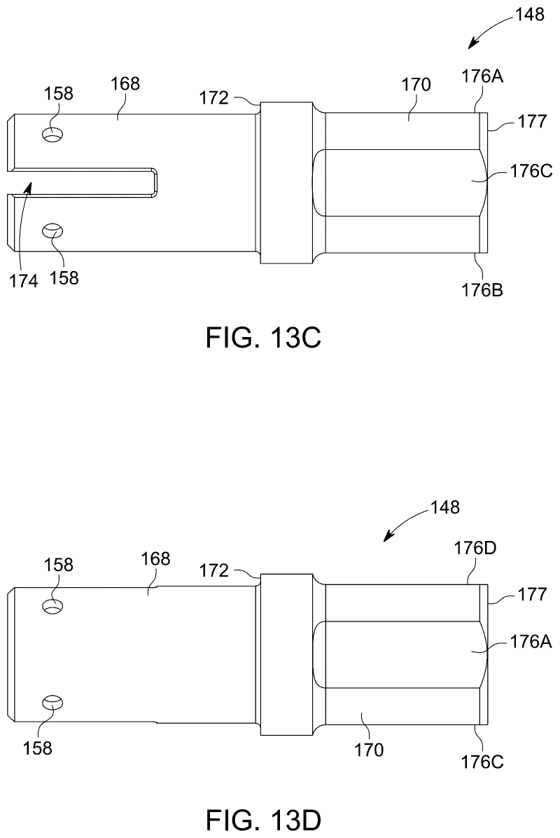

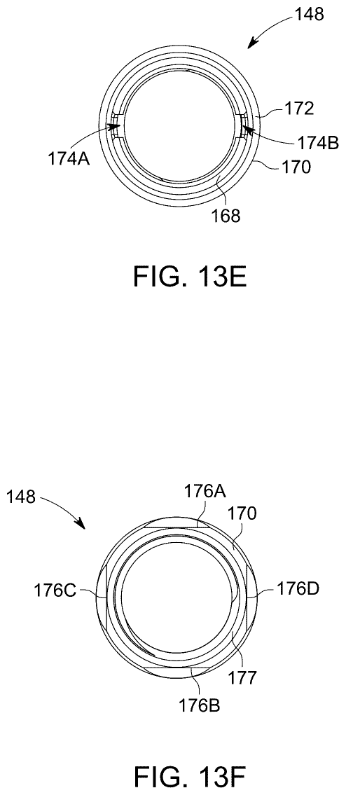

FIGS. 13A-13F illustrate the proximal sleeve 148 in isolation. The proximal sleeve 148 can have a generally cylindrical shape with a lumen passing therethrough. The proximal sleeve 148 can have a distal portion 168 and a proximal portion 170 separated by a distally facing step or shoulder 172. The shoulder 172 can serve as a surface to contact the biasing element 152 to allow that component to urge the locking sleeve 150 distally relative to the distal sleeve 146 and the proximal sleeve 148, which are coupled to one another via the pins 154. The distal portion 168 can have a diameter configured to pass through an annular space between the distal portion 163 and the proximal portion 165 of the locking sleeve 150, and to extend into the lumen of the distal sleeve 146. The distal portion 168 of the proximal sleeve 148 can extend into the distal sleeve 146 until it contacts the proximal-facing shoulder 161 formed along an inner surface of the distal sleeve and/or until the bores 156 of the distal sleeve 146 and the bores 158 formed in the distal portion 168 are aligned to receive the pins 154.

To facilitate passage of the distal portion 168 of the proximal sleeve 148 through the space between the distal portion 163 and the proximal portion 165 of the locking sleeve 150, the distal portion 168 can include one or more slots 174 extending proximally from a distal end of the proximal sleeve 148. In the illustrated embodiment, the proximal sleeve 148 includes two opposed slots 174A, 174B. The slots 174A, 174B can receive the spans 167A, 167B of the locking sleeve 150 as the proximal sleeve 148 is passed through the locking sleeve 150.

As noted above, a proximal portion of the lumen formed in the proximal sleeve 148 can include threads 144 formed thereon. As explained in more detail below, the threads 144 can engage with threads formed on a reduction handle to provide controlled reduction of a rod and/or setscrew relative to the pedicle screw 102 coupled to the reducer clip 108. A proximal portion of the proximal sleeve 148 can also include one or more features to facilitate gripping and/or grasping and/or applying torque to the reducer clip 108. For example, the proximal sleeve 148 can include one or more flat surfaces 176 on an outer proximal portion thereof to facilitate grasping the reducer clip 108 and/or applying force thereto, either directly or doing so using an intermediate tool (e.g., flat surfaces for applying a wrench or other tool, etc.). In the illustrated embodiment, the proximal sleeve 148 includes two sets of opposed flat surfaces 176A, 176B, 176C, 176D offset from one another about the longitudinal axis Ax1 of the instrument 100.

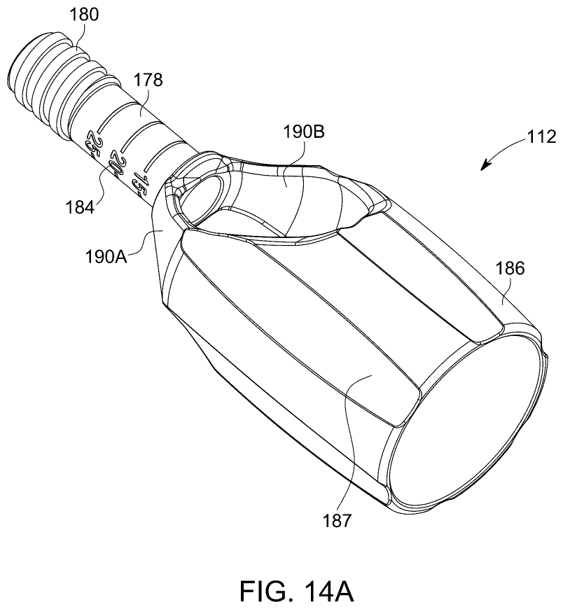

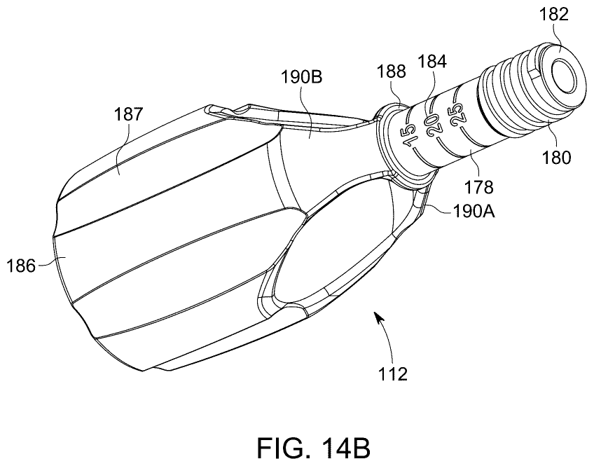

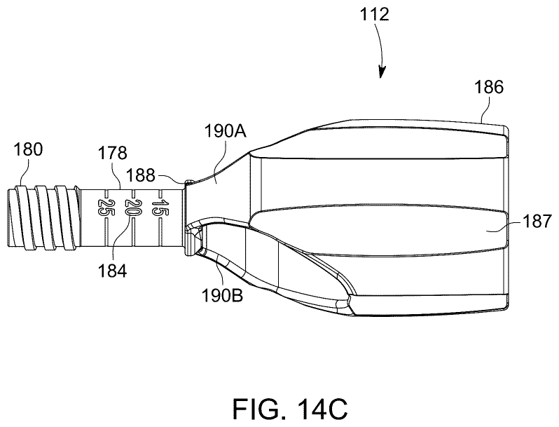

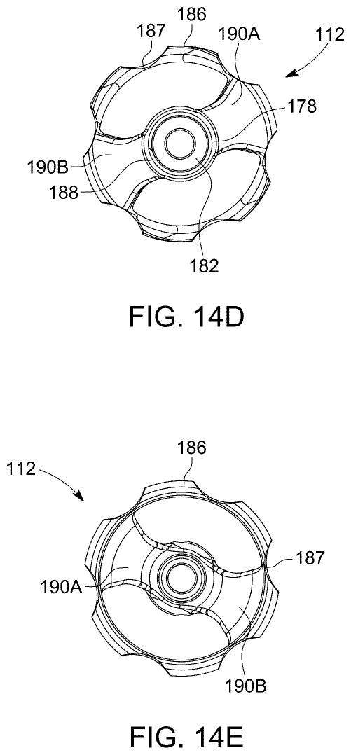

FIGS. 14A-14E illustrate the reducer handle 112 in isolation. The reducer handle 112 can include a distal portion 178 having a generally cylindrical profile with a lumen extending therethrough, e.g., to accommodate passage of the inserter-reducer shaft 110 through the reducer handle 112. The distal portion 178 can have a size configured to be received within the lumen of the reducer clip 108. In addition, the distal portion 178 can include threads 180 formed on an external surface thereof that are configured to interface with the threads 144 formed on an inner surface of the proximal sleeve 148 of the reducer clip 108 to provide for controlled movement of the reducer handle 112 relative to the reducer clip, which can thereby provide for control of reduction of a spinal rod 106 and/or setscrew 104 relative to a pedicle screw 102 coupled to the reducer clip 108.

The distal portion 178 of the reducer handle 112 can also include a blunt distal end surface 182 configured to contact the inserter-reducer shaft 110 in order to impart a distally-directed force thereto during reduction. The blunt distal end surface 182 can be substantially smooth and flat such that it is configured to bear against another flat surface on the shaft or a thrust washer or bearing disposed therebetween, to facilitate imparting an axial translation force to the shaft while allowing for relative rotation between the shaft and the reducer handle.

Still further, the distal portion 178 of the reducer handle 112 can include one or more markings 184 formed on an external surface thereof to provide feedback to a user regarding a reduction distance or amount indicated by the relative positioning of the reducer handle and the reducer clip 108. While different amounts of reduction are possible by varying the lengths of the reducer clip 108 and reducer handle 112, in the illustrated embodiment the reducer clip and reducer handle are configured to provide up to about 30 mm of rod reduction. Different marking or other feedback schemes are possible but, in the illustrated embodiment, an outer surface of the distal portion 178 of the reducer handle 112 includes markings 184 to show a distance of reduction that remains at a point where a proximal end of the reducer clip 108 aligns with a particular distance marking.

The reducer handle 112 can also include a proximal portion 186 configured for a user to grasp and apply torque thereto when performing rod reduction. The proximal portion 186 can have a generally cylindrical shape and can define a proximal-facing recess that can receive, e.g., a proximal portion of the inserter-reducer shaft 110 and a distal portion of the reducer handle 114. That is, the proximal portion 186 can form a barrel handle recess for nesting the reducer handle 112 and driver handle 114. An external surface of the proximal portion 186 can include one or more features formed thereon, such as longitudinally extending grooves 187 to aid a user in gripping and/or grasping and/or applying torque to the reducer handle 112. Any of a variety (and number) of surface features can be utilized, including features that facilitate a user directly grasping the reducer handle 112 (e.g., texturing, knurling, finger-shaped recesses, etc.), as well as using an intermediate tool (e.g., flat surfaces for applying a wrench or other tool, etc.).

The distal portion 178 and the proximal portion 186 can be separated by distally-facing step or shoulder 188 that can serve as a stop or limit on rod reduction (at least, on rod reduction provided by the reducer handle 112 and inserter-reducer shaft 110 versus, for example, what might be additionally possible using the setscrew 104 when driven by the driver handle 114). For example, the shoulder 188 can contact a proximal end of the reducer clip 108 at a point of maximum rod reduction, thereby preventing any further reduction using the reducer handle 112. Contact between the shoulder 188 and the reducer clip 108 can serve as a hard stop to prevent overreduction that might, e.g., drive the setscrew 104 into the threads 132 of the pedicle screw receiver member 118 with too much force. As noted above, the proximal portion 186 can have a generally cylindrical shape with a diameter larger than the distal portion 178. This can both facilitate easier user grasping of the reducer handle 112 and can provide a lumen or recess large enough to receive other components, such as the inserter-reducer shaft 110 and driver handle 114 coupled thereto. To accommodate the different diameters, a distal portion of the proximal portion 186 can include one or more connecting arms 190 transitioning therebetween. In the illustrated embodiment, the proximal portion 186 includes two opposed connecting arms 190A, 190B that make this transition and provide large openings therebetween. These openings can facilitate user visualization of components disposed within a recess or lumen of the proximal portion 186 and extending into the lumen of the distal portion 178 of the reducer handle 112. The openings can also facilitate cleaning and sterilization of the reducer handle 112 and other components that may be coupled thereto in an assembly.

The above-described reducer handle 112 is one example and a variety of additional embodiments are within the scope of the present disclosure (as described in more detail below). For example, in some embodiments the reducer handle can include a longitudinal slot formed therein to allow side-loading of the reducer handle onto the inserter-reducer shaft. Such a configuration can be useful, for example, in embodiments where the inserter-reducer shaft and driver handle are integrally formed and/or more permanently coupled to one another.

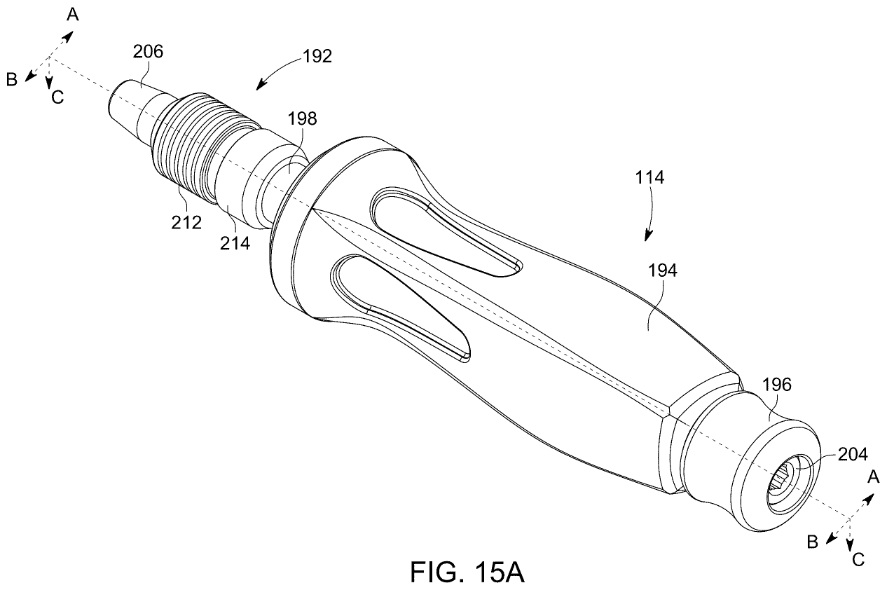

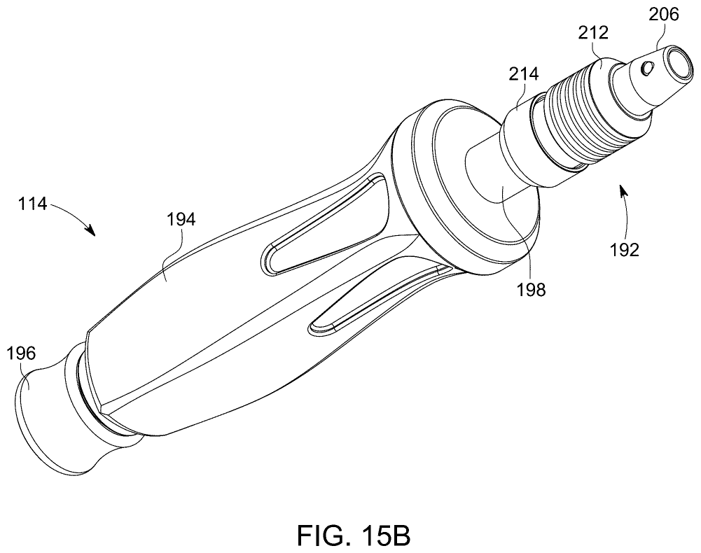

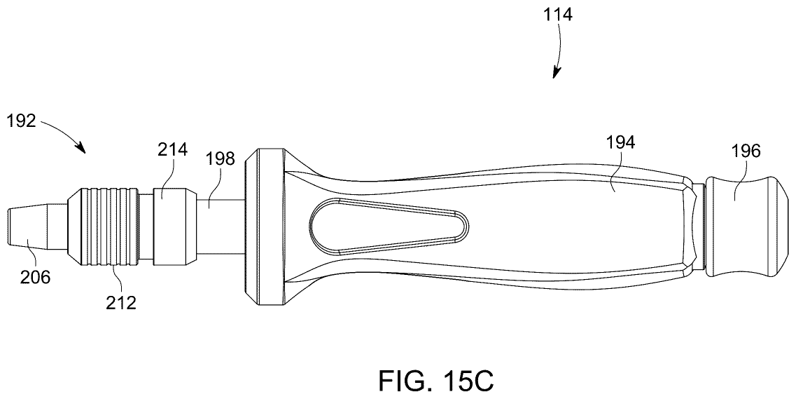

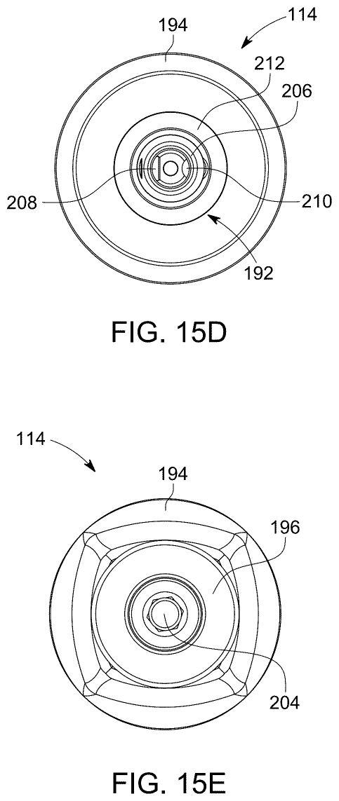

FIGS. 15A-FIG. 20 illustrate the driver handle 114 in isolation. The driver handle 114 can be configured to impart torque to the inserter-reducer shaft 110 and thereby rotate the shaft relative to the reducer clip 108 and pedicle screw 102. As explained in more detail below, the inserter-reducer shaft 110 can carry the setscrew 104 in a manner that prevents relative rotation therebetween when the setscrew is coupled to the shaft. As a result, rotation of the shaft 110 imparted by the driver handle 114 can rotate the setscrew 104 relative to the pedicle screw 102 and thereby interface the threads 138 of the setscrew with the threads 132 formed on the inner surfaces of the arms 124A, 124B and extended tabs 128A, 128B of the pedicle screw 102.

The driver handle 114 can have a generally elongate shape with an outer diameter of a size configured to be received within the lumen of the proximal portion 186 of the reducer handle 112. A distal portion of the driver handle 114 can include a quick-connect coupling 192 configured to receive a proximal end of the inserter-reducer shaft 110 and couple the driver handle thereto in a manner that selectively prevents relative movement therebetween, e.g., translation and/or rotation. While a driver handle with a modular coupling to an inserter-reducer shaft is shown, this is only an example and a variety of alternative embodiments are also within the scope of the present disclosure. These can include, for example, embodiments wherein the driver handle and inserter-reducer shaft are integrally formed as a unitary or monolithic structure, as well as embodiments wherein these components are discrete structures more permanently coupled to one another (e.g., via any of a variety of fastening techniques, including welding, adhesives, etc.) to form a single component. A proximal portion of the driver handle 114 can include one or more surfaces 194 configured to facilitate a user grasping and rotating the handle, as well as a proximal end cap 196 that can rotate relative to the remainder of the driver handle and can be utilized to steady the handle and/or apply axial force while rotating the handle.

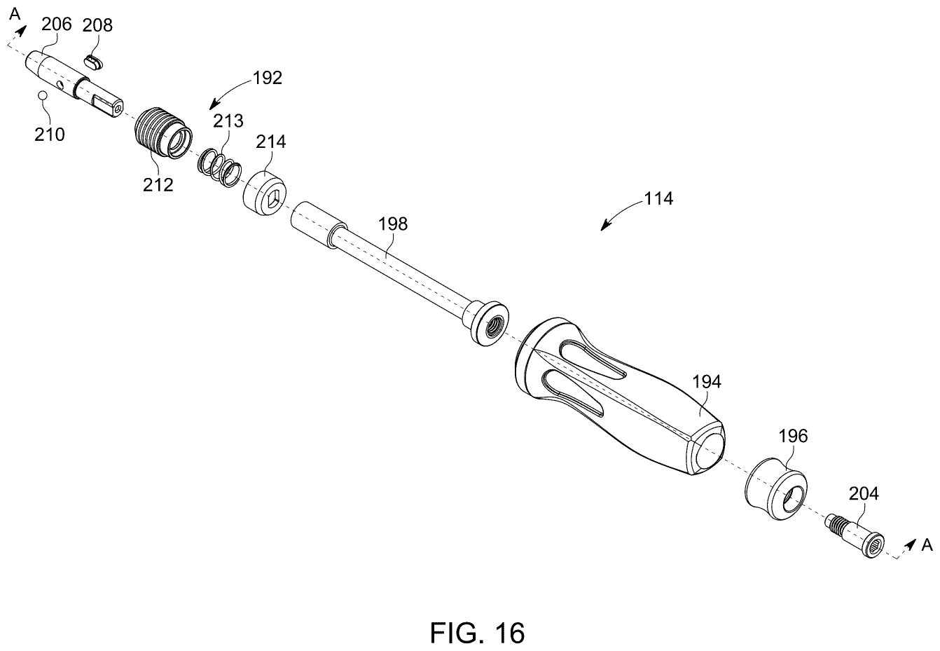

The exploded and cross-sectional views of FIGS. 16-20 illustrate the construction of the driver handle 114 in greater detail. The driver handle 114 can include a central shaft 198 having a recess 200 formed in a distal end thereof that includes at least one flat surface. The recess 200 can receive a proximal portion of the quick-connect coupling 192 that can include a complementary flat surface, such that the shaft and coupling cannot rotate relative to one another. A proximal end of the shaft 198 can include a threaded recess 202 that can receive an end screw 204. The end screw 204 can pass through a lumen of the end cap 196 and thereby retain it relative to the shaft 198 while allowing for relative rotation therebetween. The one or more surfaces 194 configured to facilitate user interaction with the driver handle 114 can be disposed over a central portion of the shaft 198. For example, a polymer handle with a lumen and said surfaces 194 formed on an exterior thereof can be disposed over the shaft 198 (or formed thereon via an over molding process, etc.).

The quick-connect coupling 192 can include a body 206 having a proximal end configured to extend into the shaft 198 and a recess formed at a distal end thereof that can be configured to receive a proximal end of the inserter-reducer shaft 110. The coupling can further include a rotation lock 208 disposed within a bore formed in a sidewall of the body 206 such that a radially-inward flat surface of the rotation lock 208 extends into the recess at the distal end of the body. The flat surface of the rotation lock 208 can be configured to contact a flat surface formed on a proximal portion of the inserter-reducer shaft 110 when it is received within the recess of the body 206, thereby preventing rotation between the shaft and the body. A radially outward surface of the rotation lock 208 can align with an outer surface of the body 206. The rotation lock 208 can be securely coupled to the body in a variety of manners, e.g., welding, adhesive, interference fit, etc. The coupling can also include a ball 210 disposed within a bore formed in the sidewall of the body 206, e.g., opposite the rotation lock 208, such that the ball can move radially inward or outward to selectively extend into the recess at the distal end of the body. The ball can serve as a translation lock when advanced radially inward into the recess and disposed within an at least partial circumferential groove formed in the outer surface of a proximal portion of the inserter-reducer shaft 110.

A distal housing 212 can surround the body 206 and ball 210. The distal housing 212 can prevent the ball 210 from falling out of the body 206 and can selectively control a radial position of the ball via a lumen formed in the distal housing with a proximally tapering diameter. The diameter of the lumen can be configured such that, when a distal portion of the lumen with a larger diameter is disposed over the ball 206, the ball can move radially outward to minimize extension into the recess of the body 206. Conversely, when a proximal portion of the lumen with a smaller diameter is disposed over the ball 206, the ball can move radially inward to extend into the recess of the body. If this is done when the inserter-reducer shaft 110 is disposed within the recess, such that a circumferential groove formed therein is aligned with the ball, the ball can selectively lock the shaft against axial movement relative to the driver handle 114.

The quick coupling 192 can further include a proximal housing 214 disposed around the body 206 and a portion of the distal housing 212. The proximal housing 214 can define an annular recess around the body 206 that can house a biasing element 213, such as a coil spring. The biasing element 213 disposed between the distal housing 212 and the proximal housing 214 can distally bias the distal housing 212. Given the configuration of the distal housing 212 described above, the biasing element 213 can bias the quick-connect coupling 192 into a locked configuration in which the ball 210 is forced radially-inward such that it can lock the driver handle 114 to the inserter-reducer shaft 110. To separate the two components, a user can proximally withdraw the distal housing 212 relative to the proximal housing 214 (and remainder of the driver handle 114) to allow the ball 210 to move radially outward such that the driver handle 114 and inserter-reducer shaft 110 can be axially translated relative to one another.

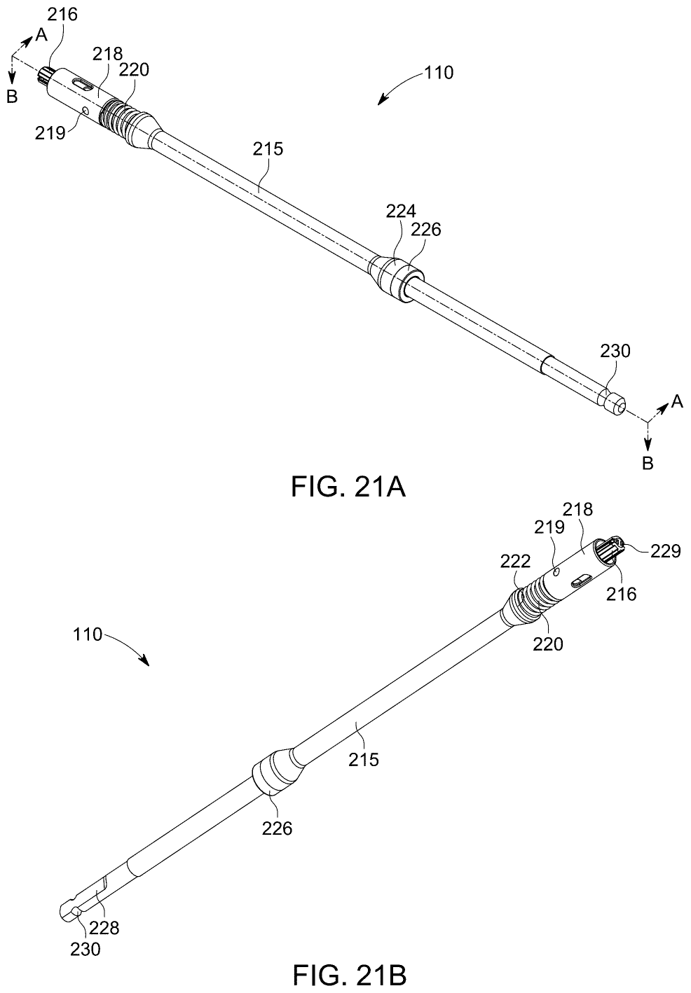

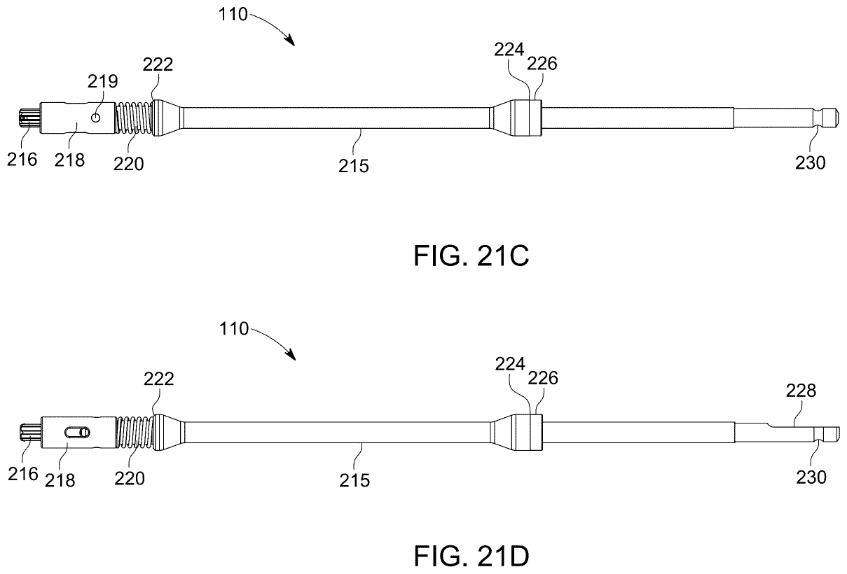

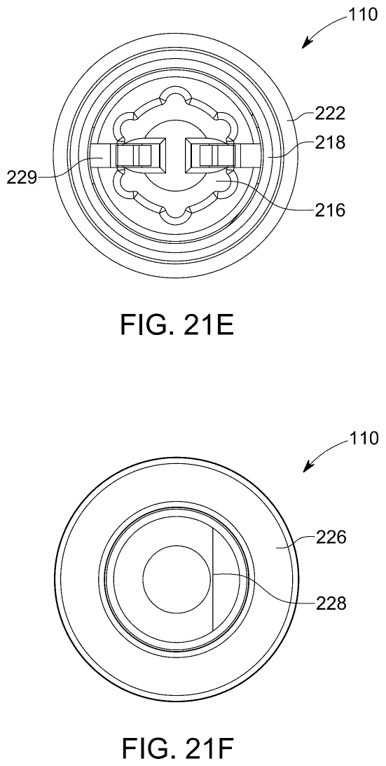

FIGS. 21A-29 illustrate the inserter-reducer shaft 110 in isolation. The shaft 110 can include an elongate body 215 and a distal portion 216 configured to extend through a lumen formed in the setscrew 104 and rotationally drive the setscrew. The shaft 110 can further include a sleeve 218 disposed around the shaft over at least a distal portion thereof, as well as a biasing element 220 disposed proximal to the sleeve and configured to urge the sleeve distally relative to the remainder of the shaft. The biasing element 220 can be, for example, a coil spring disposed around the elongate body 215. The shaft can further include a distal-facing shoulder 222 formed proximal to the distal portion 216 and the sleeve 218, such that the biasing element 220 can be disposed between the sleeve 218 and the distal-facing shoulder 222, which can allow the biasing element 220 to bias the sleeve 218 distally relative to the elongate body 215.

While the sleeve 218 can be biased distally relative to the elongate body 215, its range of motion can also be constrained such that it cannot translate relative to the elongate body beyond a distal end of the distal portion 216 that is configured to rotationally drive the setscrew 104. This can be accomplished in a variety of manners. In the illustrated embodiment, for example, a pin 219 can extend through a bore 221 formed in the sleeve 218 and an elongate slot 223 formed in the elongate body 215, such that the pin traveling in the elongate slot defines the range of motion of the sleeve relative to the elongate body. Therefore, the desired range of motion can be controlled using the axial lengths of the elongate slot 223 and the sleeve 218, as well as their placement along the shaft 110.

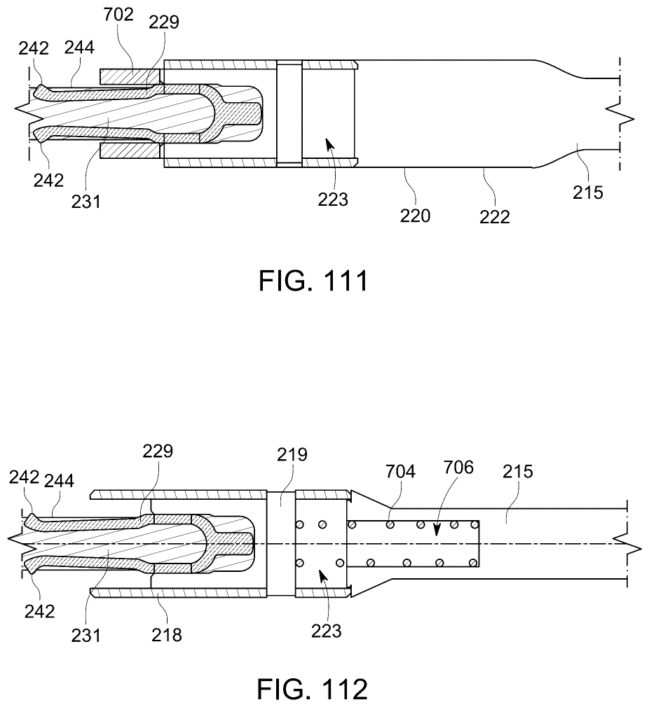

The use of a translating sleeve 218 and biasing element 220 is one example and a variety of alternative configurations are also within the scope of the present disclosure. For example, FIG. 111 illustrates one alternative embodiment that replaces the translating sleeve 218 and biasing element 220 with a biasing element 702 in the form of a ring of elastomeric material. FIG. 111 is otherwise labeled with components similar to FIG. 28 to show context, but includes the elastomeric ring 702 disposed around a distal portion of the shaft. The ring 702 can be positioned proximal to a setscrew 104 and can be compressible, such that the elastomeric ring 702 can perform a similar function as the biased sleeve 218 described above.

FIG. 112 illustrates another alternative embodiment in which the biasing element 220 is a spring disposed within the elongate body 215 rather than around the shaft, as shown in FIG. 28. As with FIG. 111, FIG. 112 is otherwise labeled with components similar to FIG. 28 to show context, but includes biasing element 704 in the form of a coil spring disposed within a recess 706 formed in the elongate body 215. The spring 704 can act on the pin 219 that moves with the sleeve 218 in order to achieve the same distal biasing of the sleeve.

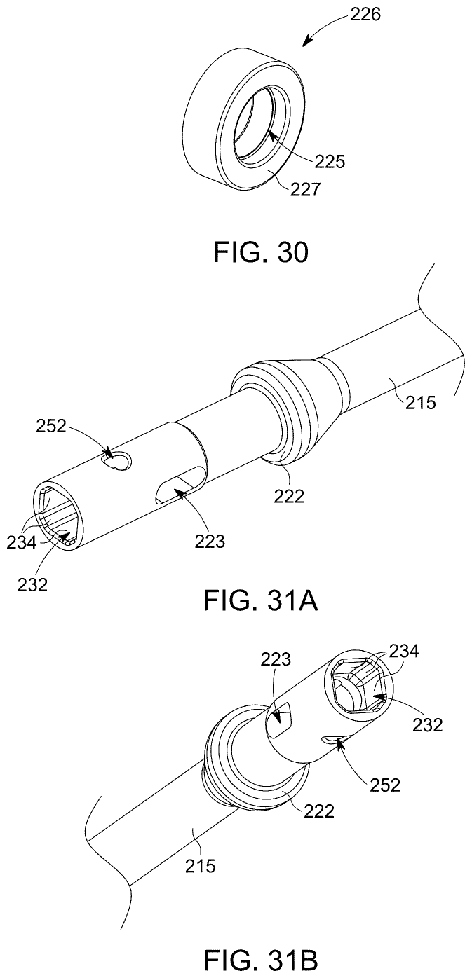

Returning to the embodiment of FIGS. 21A-29, the elongate body 215 can further include a proximal-facing shoulder 224 formed proximal to the distal-facing shoulder 222. The proximal-facing shoulder 224 can provide a surface configured to engage with the distal end of the reducer handle 112 during a reduction operation. Accordingly, the distal end surface 182 of the reducer handle 112 can apply axial and distally directed reduction forces against, and rotate over, the proximal-facing shoulder 224 as the reducer handle is threaded into the reducer clip 108. To facilitate this engagement, the inserter-reducer shaft 110 can include a thrust bearing 226 disposed around the elongate body 215 proximal to the proximal-facing shoulder 224, though this feature can be omitted in some embodiments. FIG. 30 illustrates the thrust bearing 226 in greater detail, including its generally cylindrical shape with lumen 225 and opposed bearing surfaces 227 (only one visible in the figure). The thrust bearing 226 can have a variety of forms. For example, it can be a unitary or monolithic component, such as a polymer or metal (e.g., certain grades of stainless steel, such as nitronic 60, etc.) washer with good wear characteristics for use in such a configuration, or a mechanical assembly, such as a cylindrical ball bearing with opposed bearing washers trapping a plurality of ball bearings therebetween.

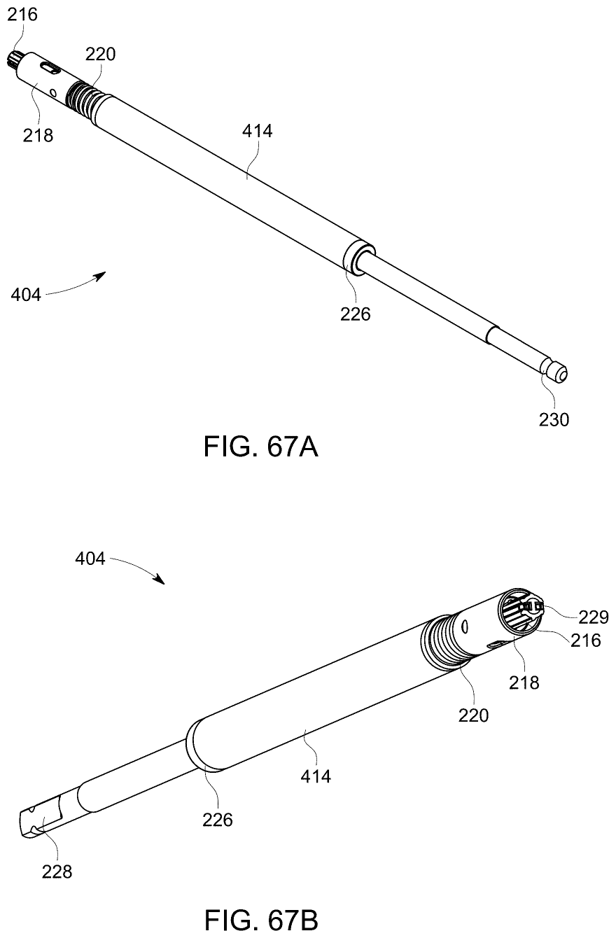

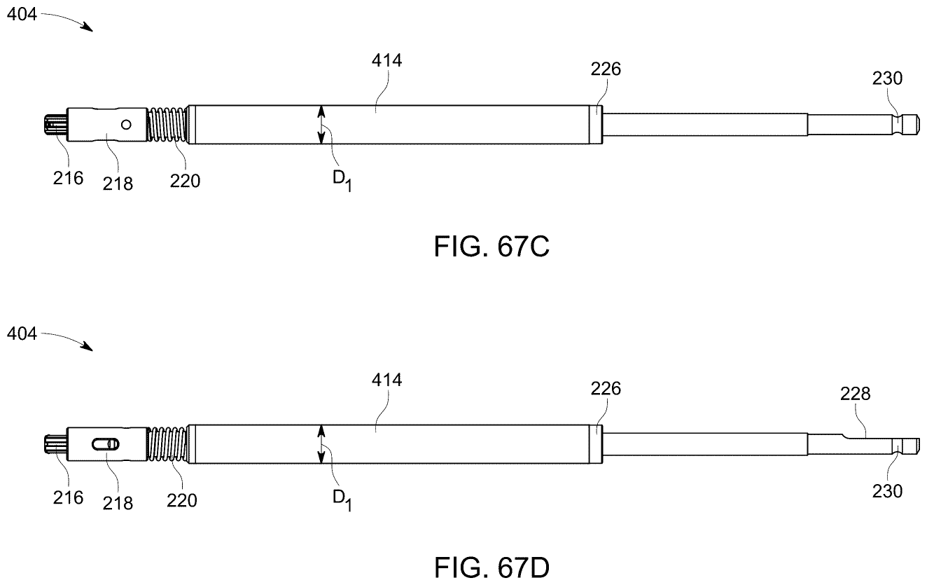

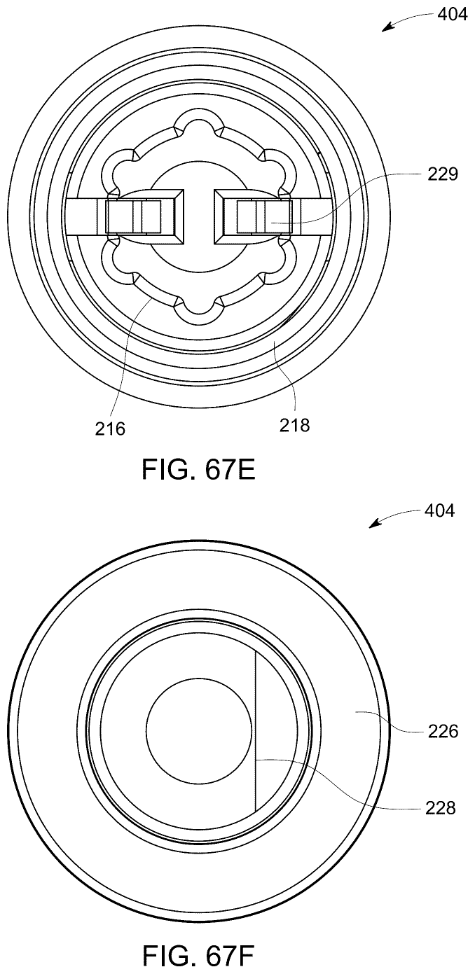

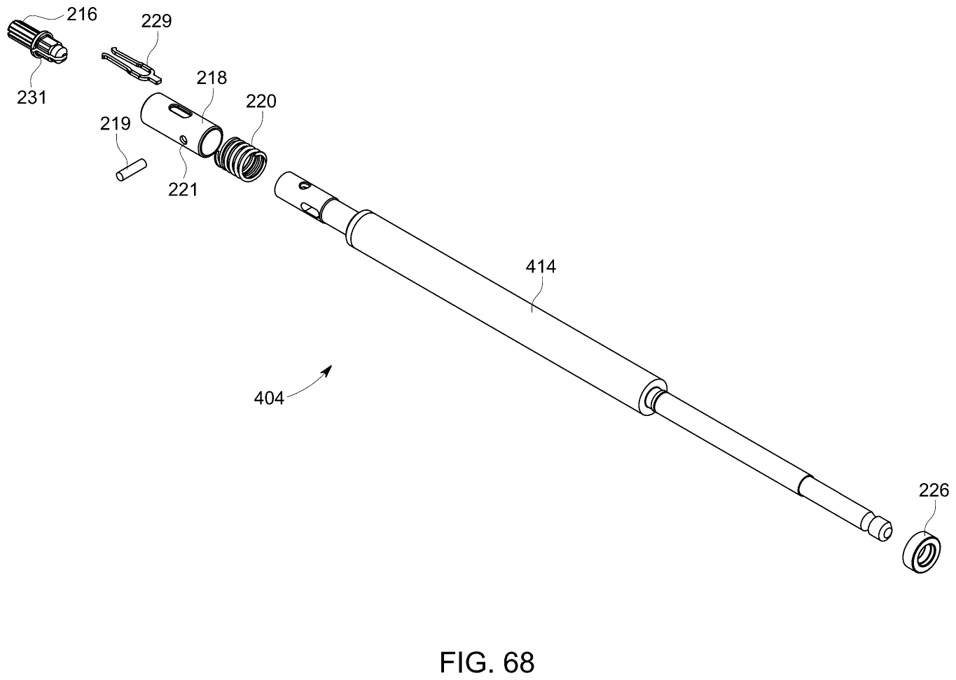

The shoulders 222, 224 and other components of the shaft 110 can have a variety of diameters. In some embodiments, an outer diameter of the distal-facing shoulder 22 and an outer diameter of the proximal-facing shoulder 224 can be equal. Further, in some embodiments, a portion of the elongate body 215 extending between the distal-facing shoulder 222 and the proximal-facing shoulder 224 can have an outer diameter that is less than a diameter of one or more of the shoulders 222, 224. In other embodiments, e.g., as shown in FIGS. 67A-69F described below, a portion of the elongate body extending between a distal-facing shoulder and a proximal-facing shoulder can have an outer diameter equal to the diameter of one or more of the shoulders.

A proximal portion of the inserter-reducer shaft 110 can include one or more flat surfaces 228 formed therealong. The surface 228 can be configured to contact, for example, the rotation lock 208 of the driver handle 114 to couple the two components in a manner that prevents relative rotation therebetween. The proximal portion of the inserter-reducer shaft 110 can also include one or more at least partial circumferential grooves 230 formed therein. The proximal portion of the inserter-reducer shaft 110 containing these features can be positioned proximal to the proximal-facing shoulder 224 and/or the thrust bearing 226.

The distal portion 216 that is configured to rotationally drive the setscrew 104 can have a cross-sectional shape that is complementary to a shape of the drive feature 136 of the setscrew. In addition, one or more features can be added to help retain a setscrew 104 when disposed over the distal portion 216. In the illustrated embodiment, a spring clip 229 can be utilized to selectively retain a setscrew 104 on the shaft 110 until sufficient force is applied to overcome the spring clip and pass the setscrew over a distal end thereof.

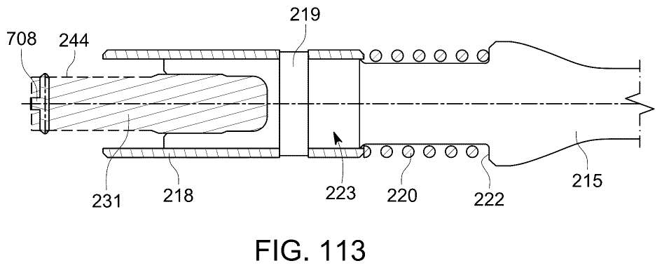

The use of a spring clip 229 to retain a setscrew 104 on the inserter-reducer shaft is one example and a variety of alternative configurations are also within the scope of the present disclosure. For example, FIG. 113 illustrates one alternative embodiment that replaces the spring clip 229 with a retainer 708 formed of an elastomeric material. FIG. 113 is otherwise labeled with components similar to FIG. 28 to show context, but includes the retainer 708 disposed along a distal portion of the shaft. The retainer 708 can include one or more flexible protrusions that can interfere with the setscrew 104 drive feature (two such protrusions are shown in the figure). The protrusions (and the elastomeric retainer generally) can be compressible such that it can provide similar functionality to the spring clip 229 described above. Moreover, the figure shows the retainer 708 flush with a distal tip of the inserter-reducer shaft. Such a configuration can be achieved using an insert molding process (e.g., where the inserter-reducer shaft is placed into a mold and the elastomeric retainer 708 is molded in place on the end of the shaft). The distal tip of the shaft can be modified to provide a molding gate location in such an embodiment. For example, the shaft can have one or more cross holes drilled therein, as well as a central hole. The empty volume can be filled with liquid silicone rubber or a similar elastomer to form the retainer 708. In some embodiments, the elastomeric setscrew retention of FIG. 113 can be combined with the elastomeric setscrew biasing shown in FIG. 111.

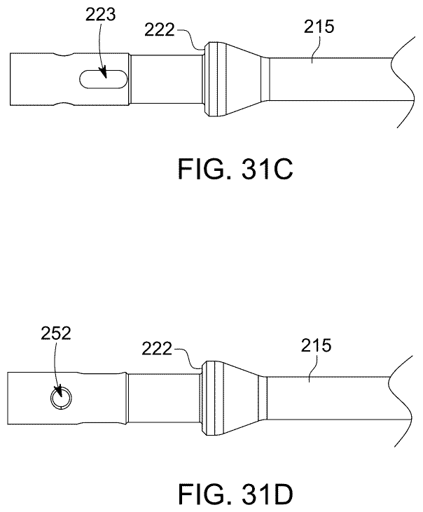

Generally speaking, it can be possible to form the various components disclosed herein in a variety of manners and using either unitary, monolithic structures or assemblies of various modular components. In the illustrated embodiments, for example, the distal portion 216 configured to rotationally drive the setscrew 104 can be formed on an insert 231 that is separate from the elongate body 215. In such an embodiment, the elongate body 215 can include a distal-facing recess 232 configured to receive a proximal portion of the insert 231. The two components can be coupled together in any of a variety of manners to prevent unintended separation thereof (e.g., interference fit, adhesive bonding, welding, mechanical fastening, etc.). Further, they can be coupled together in a manner the prevents relative rotation therebetween (e.g., utilizing one or more flat surfaces that contact one another, a through-bolt or pin arrangement, etc.). For example, the distal-facing recess 232 formed in the elongate body 215 can include one or more flat surfaces 234 configured to contact one or more flat surfaces of the insert 231 that is received in the recess. In the illustrated embodiment, the distal-facing recess 232 can include a plurality of flat surfaces 234, as shown particularly in FIGS. 31A and 31B. The surfaces 234 can be configured to contact a corresponding plurality of flat surfaces 236 formed on a proximal portion 238 of the insert 231.

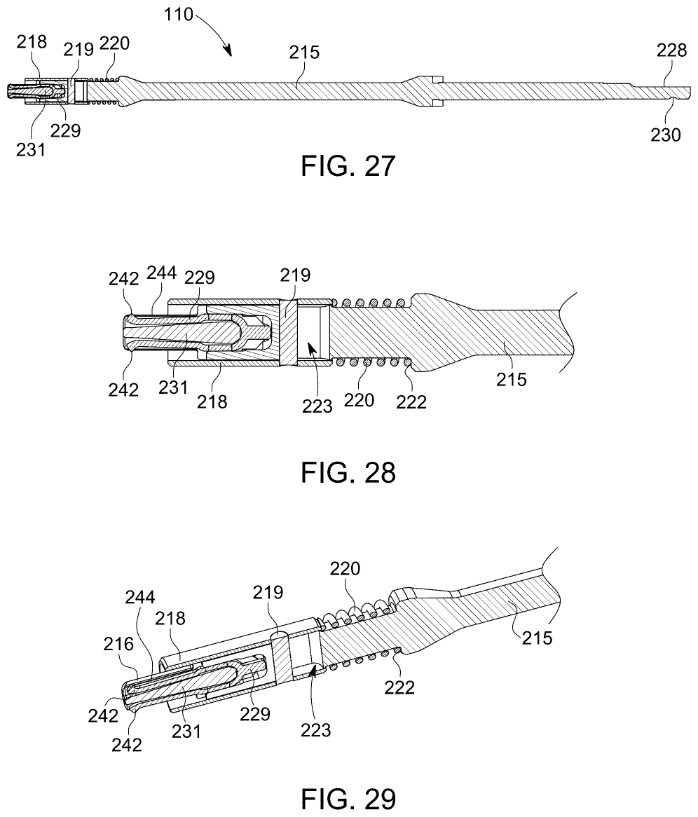

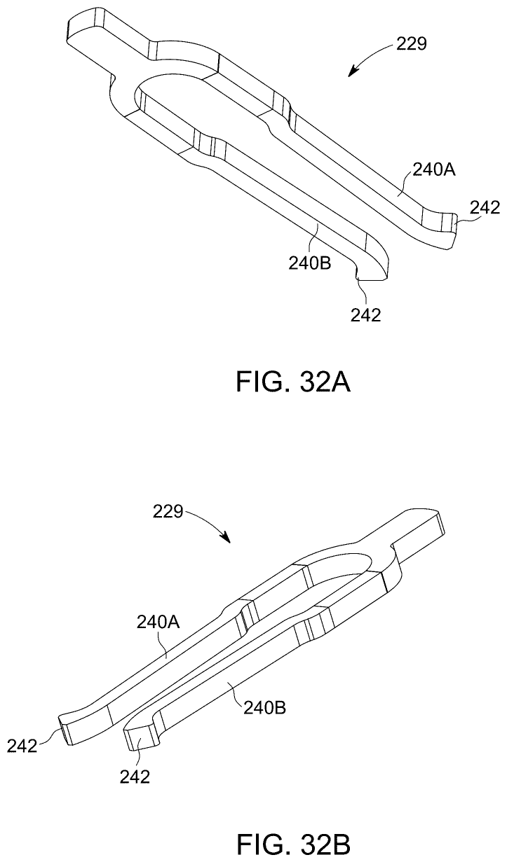

FIGS. 32A and 32B illustrate the spring clip 229 in isolation. The spring clip 229 can have a generally “U” or “Y” shaped profile with opposed arms 240A, 240B (though, in other embodiments, a single-arm spring clip can be utilized). Distal ends of the arms 240A, 240B can include a protrusion, hook, or other feature 242 that can be configured to contact a setscrew 104 and prevent distal translation thereof along the distal portion 216 of the shaft 110 until sufficient force is applied to cause the opposed arms 240A, 240B of the spring clip 229 to deform radially-inward toward one another and allow the protrusions 242 to pass through the drive feature lumen 126 of the setscrew 104. As shown in FIGS. 27-29, showing cross-sectional views of the distal portion of the inserter-reducer shaft 110, the protrusions 242 of the spring clip 229 can be positioned near a distal end of the distal portion 216 and the setscrew 104 can translate freely along the distal portion 216 when positioned proximal to the protrusions 242.

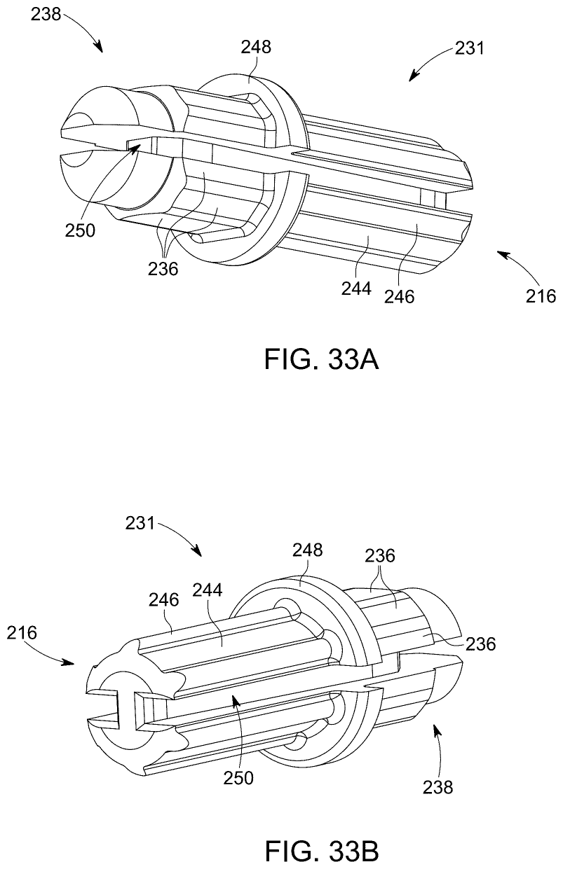

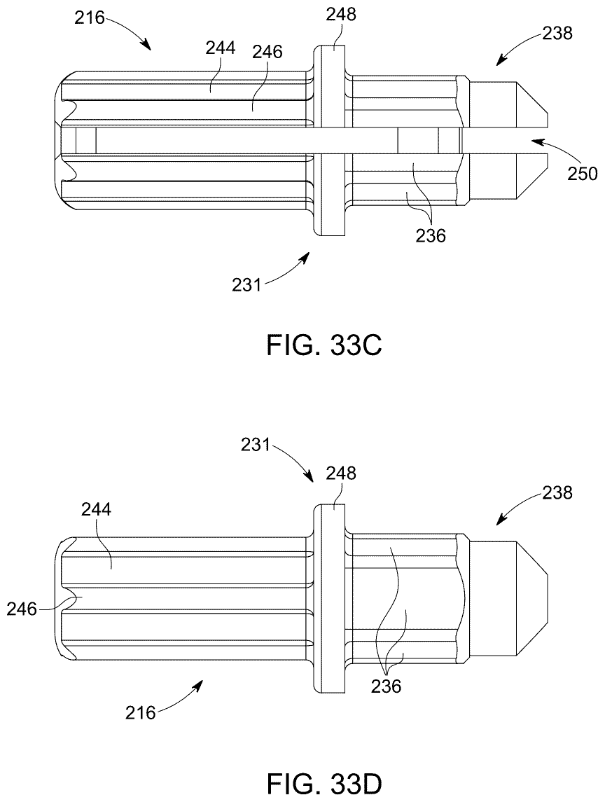

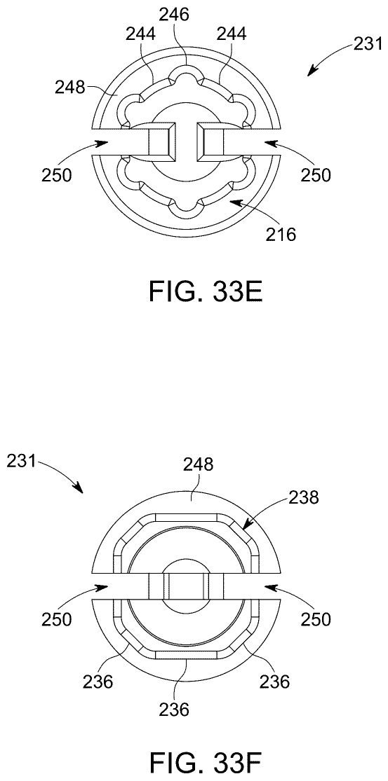

FIGS. 33A-33F illustrate the insert 231 in isolation. As noted above, the insert 231 can include the distal portion 216 configured to seat and rotationally drive the setscrew 104. To this end, the cross-sectional profile of the distal portion 216 can have a shape that is complementary to the drive feature 136 of the setscrew 104, e.g., including a hexagonal arrangement of surfaces 244 disposed around a circumference thereof with curved protrusions 246 disposed between adjacent flat surfaces. In other embodiments, any of a variety of different shapes can be utilized, including, e.g., torx, etc.

A flange 248 can separate the distal portion 216 from the proximal portion 238 configured to be inserted into the recess 232 of the elongate body 215. In some embodiments, the flange 248 can serve as a proximal stop to prevent a setscrew 104 disposed over the distal portion 216 from translating proximally beyond the flange. Though, in certain embodiments, the setscrew 104 would not be able to reach the flange 248 before its proximal movement would be stopped by another component, such as the full insertion of the reducer handle 112 against the reducer clip 108 and/or the full reduction of the rod 106 into the receiver member 118. Regardless, the flange 248 can serve as an insertion stop for the insert 231 relative to the elongate body 215, as it prevents over-insertion and provides feedback to a user during assembly when the flange contacts a distal end surface of the elongate body.

The proximal portion 238 of the insert 231 can include a cross-sectional profile that is complementary to the cross-sectional profile of the recess 232 in the elongate body 215. For example and as noted above, the proximal portion 238 can include one or more flat surfaces 236 configured to contact one or more corresponding flat surfaces that form the sidewalls of the recess 232. Any of a variety of different numbers, shapes, and configurations of such surfaces can be utilized in different embodiments.

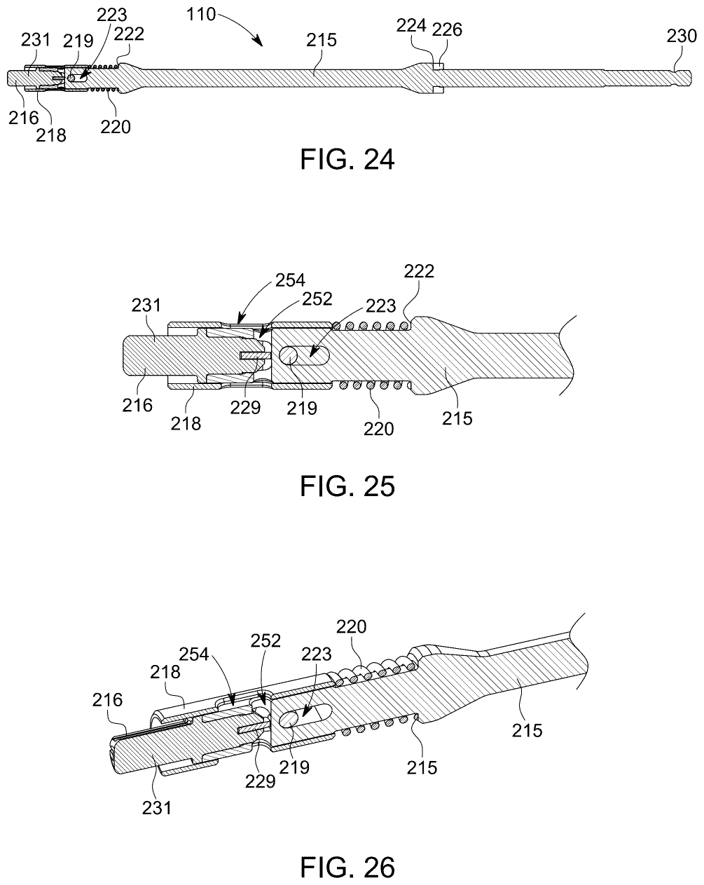

The insert 231 can also include a longitudinally extending slot 250 formed therein that can be configured to receive the spring clip 229 therein. For example, the slot 250 can extend longitudinally along opposed sides of the insert 231 and around a proximal end thereof, such that the “U” or “Y” shaped spring clip 229 can be loaded into the insert 231 by distally translating the spring clip 229 from a proximal position relative to the insert. When the insert 231 is disposed within the distal-facing recess 232 of the elongate body 215, the two components can trap the spring clip in a desired position. The cross-sectional views of FIGS. 24-29 show the spring clip 229 assembled to the insert 231, and the insert 231 disposed in the distal-facing recess 232 of the elongate body 215. FIGS. 27-29, in particular, show how the protrusions 242 at the ends of each arm 240A, 240B of the spring clip 229 extend above the plane of the surface 244 such that they would interfere with distal translation of a setscrew 104 off the distal portion 216 of the insert 231. This interference can be overcome, however, with sufficient force to urge the arms 240A, 240B radially inward toward one another (i.e., further into the slot 250 of the insert toward a longitudinal axis thereof). Once sufficiently moved in this manner, the protrusions 242 can pass through the central drive feature lumen 136 of the setscrew 104 and the setscrew can translate distally off the distal portion 216 of the insert 231.

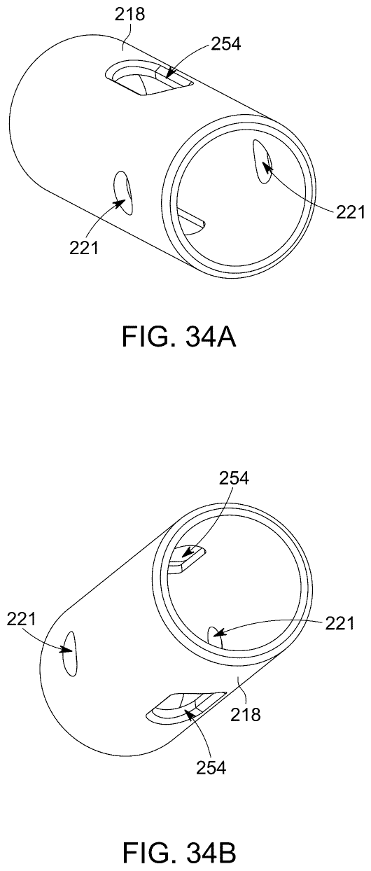

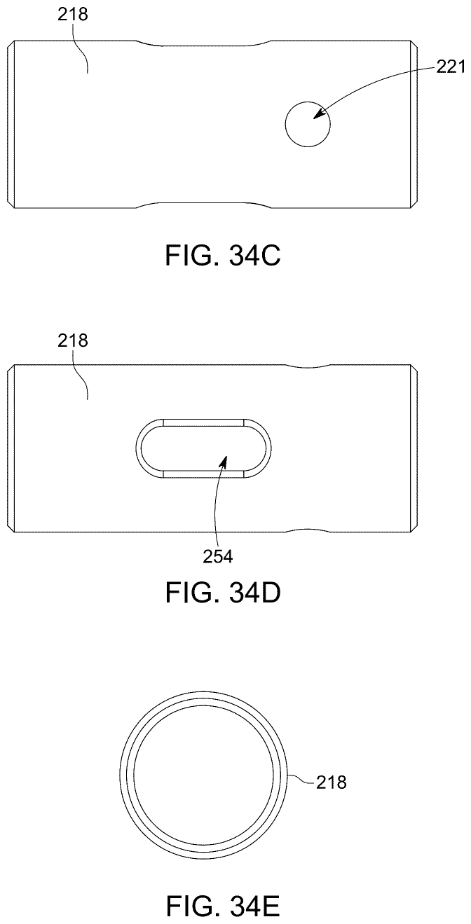

In order to facilitate access and/or cleaning of these components, the elongate body 215 can include a bore 252 that intersects a proximal portion of the recess 232. Further, the sleeve 218 can include an elongate slot 254 configured to align with the bore 252 at any point along the range of motion of the sleeve relative to the elongate body 215. These features can be seen in the cross-sectional views of FIGS. 24-26, as well as the isolated views of the elongate body in FIGS. 31A and 31B, and the isolated view of the sleeve 218 in FIGS. 34A-34E.

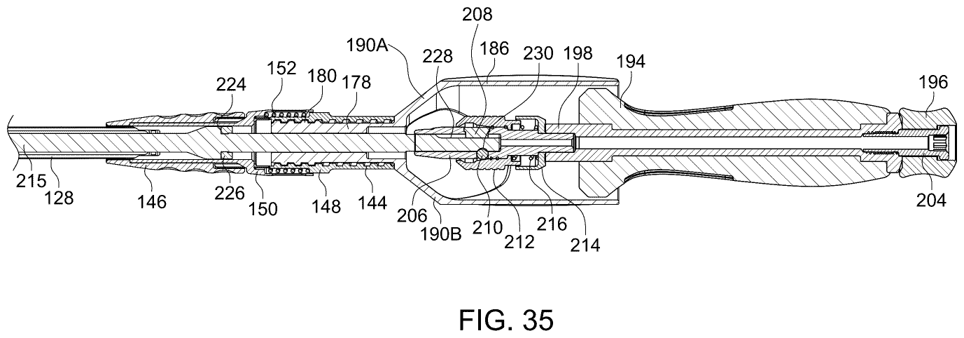

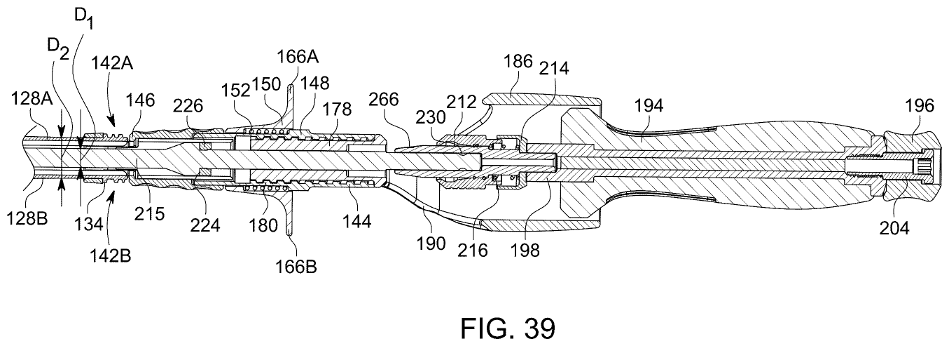

FIGS. 35-42 illustrate cross-sectional views of various portions of the assembly of FIG. 1A and instrument 100. In particular, FIGS. 35-38 show cross-sectional views of the proximal and distal portions of the assembly in a first plane taken along the lines A-A in FIG. 1A, while FIGS. 39-42 show cross-sectional view of the proximal and distal portions of the assembly in a second plane taken along the lines B-B in FIG. 1A that is offset 90 degrees from the first plane. These figures can serve to orient the various features described above in connection with the figures showing the components in isolation.

FIGS. 43-52 illustrate one example method of utilizing the instrument 100 according to the present disclosure. As shown in FIG. 43, for example, a rod 106 (e.g., an implanted spinal fixation rod that would extend farther than is illustrated in the figure) can be captured between the extended tabs 128 of the pedicle screw receiver member 118 and the reducer clip 108 can be brought near to the proximal end of the extended tabs 128A, 128B of the receiver member and aligned with a longitudinal axis thereof. Note that these operations can be performed in either order, e.g., by capturing the rod first and then coupling the reducer clip to the pedicle screw, or by first coupling the reducer clip to the pedicle screw and then capturing the rod (e.g., by passing the rod into the space between the extended tabs 128A, 128B of the pedicle screw receiver member from the side relative to the receiver member rather than from above along a longitudinal axis of the receiver member).

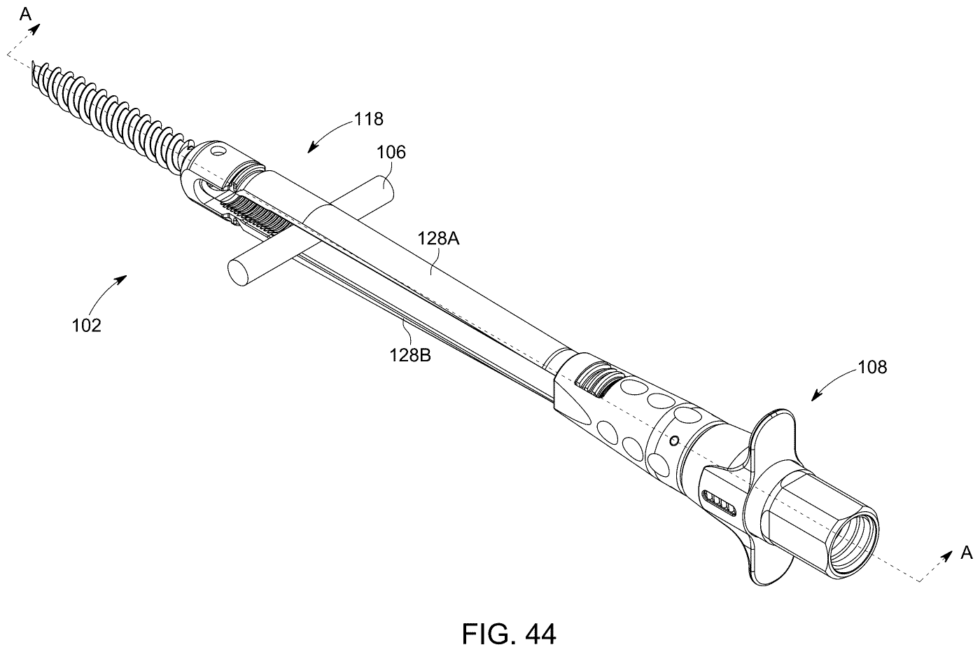

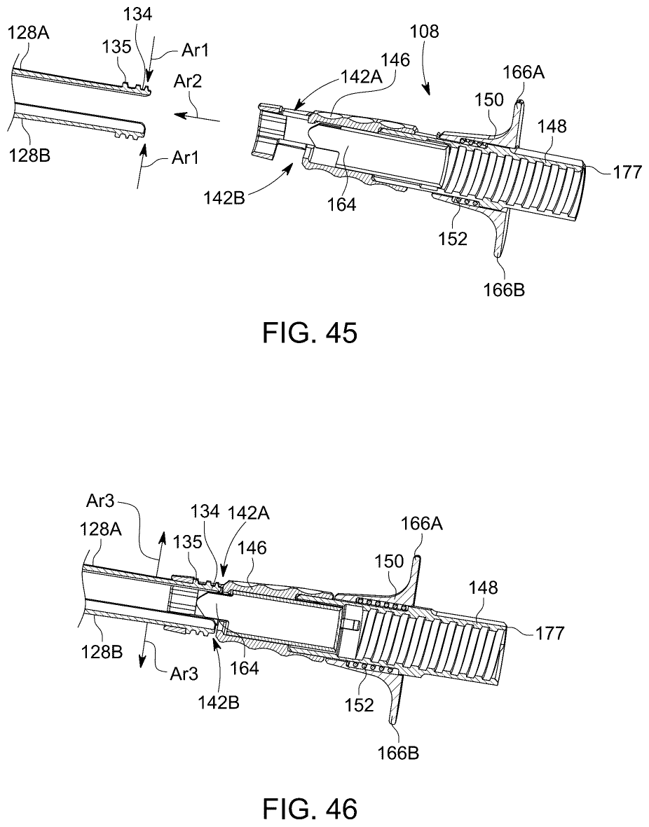

The reducer clip 108 can be coupled to the pedicle screw 102 and, more particularly, to a proximal end of the extended tabs 128A, 128B of the receiver member 118, as shown in FIG. 44. This can be accomplished by squeezing a proximal portion of the extended tabs 128A, 128B together (i.e., radially inward toward a longitudinal axis of the pedicle screw 102, as shown by arrows Ar1 in FIG. 45) and advancing a distal portion of the reducer clip 108 over a proximal portion of the extended tabs in the direction of arrow Ar2 in FIG. 45). During assembly, the reducer clip 108 can be held like a syringe with a user placing a finger distal to each tab 166A, 166B extending radially away from the locking sleeve 150. A user can also provide a distally-directed counter-force (e.g., using a thumb or palm) to the proximal end surface 177 of the reducer clip 108 to pull back the reducer clip locking sleeve 150 proximally relative to the remainder of the reducer clip, as shown in FIG. 45. Coupling and separating the reducer clip 108 and the pedicle screw 102 makes use of the flexible nature of the extended tabs 128A, 128B that, in some embodiments, can deflect along their length and/or about the reduced-thickness material 130A, 130B that connects the tabs to the remainder of the receiver member 118. A proximal portion of the extended tabs 128A, 128B can remain collapsed radially inward toward one another until a proximal flange 135 and/or external threads 134 formed thereon are aligned with the recesses 142A, 142B of the reducer clip 108. At this point, the extended tabs 128A, 128B can move radially outward away from one another in the direction of arrows Ar3 in FIG. 46 and return to their resting positions with the threads 134 and/or flange 135 disposed within the recesses 142A, 142B such that the reducer clip 108 cannot be separated from the pedicle screw 102. This can give the user confirmation that the reducer clip 108 is correctly installed. The locking sleeve 150 can then be released, allowing the biasing element 152 to urge the locking sleeve distally relative to the remainder of the reducer clip 108 and the pedicle screw 102. The pair of locking tabs or blades 164 formed at the distal end of the locking sleeve 150 can translate distally into a space between the opposed extended tabs 128A, 128B. In this distal position, the locking tabs 164 reside between the two extended tabs 128A, 128B, thereby preventing the extended tabs from collapsing or moving radially inward toward one another. This effectively prevents the reducer clip 108 from disassembling with respect to the pedicle screw 102. As explained in more detail below, the reduction instruments can be threaded into the female threads 144 on the reducer clip 108.

In other embodiments, the reducer clip 108 can be coupled to the pedicle screw 102 in a similar manner but where a user does not translate the locking sleeve 150 using the tabs 166A, 166B. Instead, after axially aligning the reducer clip 108 and the extended tabs 128A, 128B, a user can squeeze the extended tabs together and push the reducer clip over the extended tabs without retracting the locking sleeve. The extended tabs 128A, 128B would contact a distal portion of the locking sleeve and move it proximally with respect to the remainder of the reducer clip 108 until the threads 134 and/or flange 135 align with the recesses 142A, 142B. When the extended tabs 128A, 128B are released and expand radially outward, the locking sleeve 150 would travel distally into its locked position from the force of the biasing element 152.

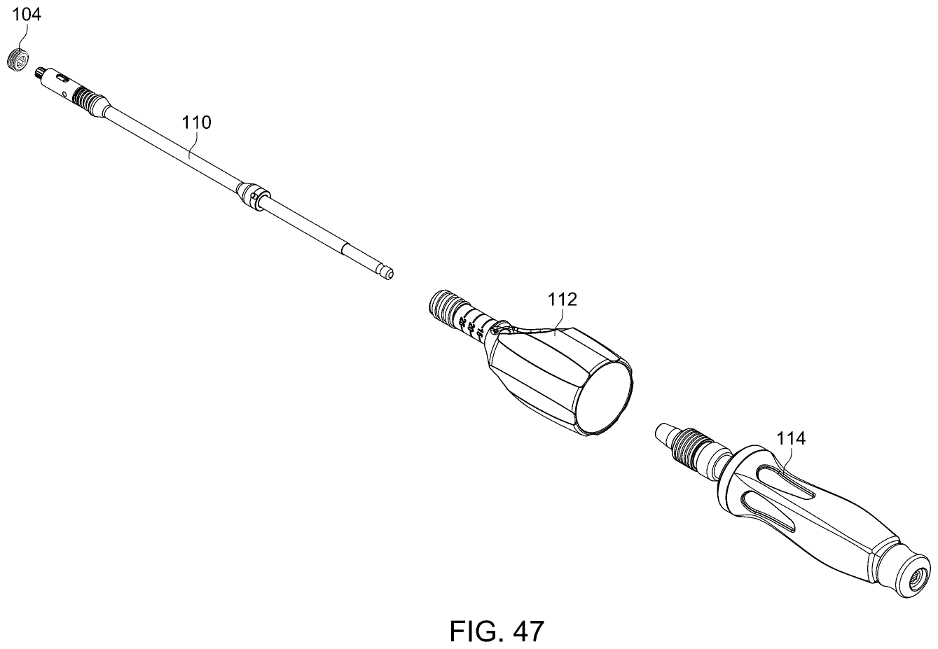

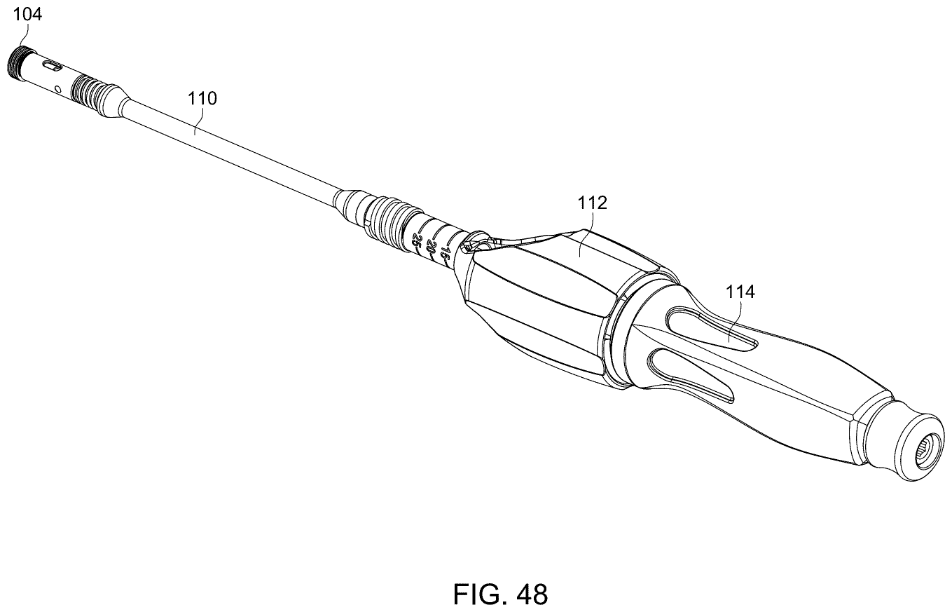

Turning to the reduction instrument aside from the reducer clip 108, FIGS. 47 and 48 illustrate the assembly thereof in anticipation of insertion through the reducer clip 108. In particular, a setscrew 104 can be loaded onto a distal end of an inserter-reducer shaft 110 by passing a distal portion 216 of the shaft through a drive feature lumen 136 of the setscrew 104. This can involve passing the setscrew 104 proximally over projections 242 of a spring clip 229. Once the projections 242 are disposed distal to the setscrew 104, they can help retain the setscrew relative to the shaft 110 until sufficient force is applied to move the setscrew 104 distally over the projections. Furthermore, the setscrew 104 can be advanced proximally over the distal portion 216 of the shaft 110 until the setscrew contacts the distally biased sleeve 218 disposed around a portion of the shaft.

A proximal end of the inserter-reducer shaft 110 can be inserted into a distal end of the reducer handle 112 and through the lumen extending therethrough. The shaft 110 can be inserted proximally through the lumen of the reducer handle 112 until a distal end surface 182 of the reducer handle contacts a thrust bearing 226 and/or proximal-facing shoulder 224 of the shaft.

Further, the driver handle 114 can be coupled to the proximal end of the inserter-reducer shaft 110 extending proximally through the lumen of the reducer handle 112. This can be accomplished, for example, by a user proximally withdrawing the distal housing 212 of the quick-connect coupling 192 and inserting a proximal portion of the shaft 110 into the recess formed at the distal end of the body 206 of the coupling. The user can then release the distal housing 212 and allow it to move distally from the biasing force of spring 213, which can cause the distal housing to lock the ball 210 into the groove 230 to prevent separation of the shaft 110 and driver handle 114. As shown in the assembled view of FIG. 48, this can mean that a proximal portion of the shaft 110 and a distal portion of the driver handle 114 can be disposed within a proximal portion of the reducer handle 112, and a proximal portion of the driver handle can extend proximally beyond a proximal end of the reducer handle.

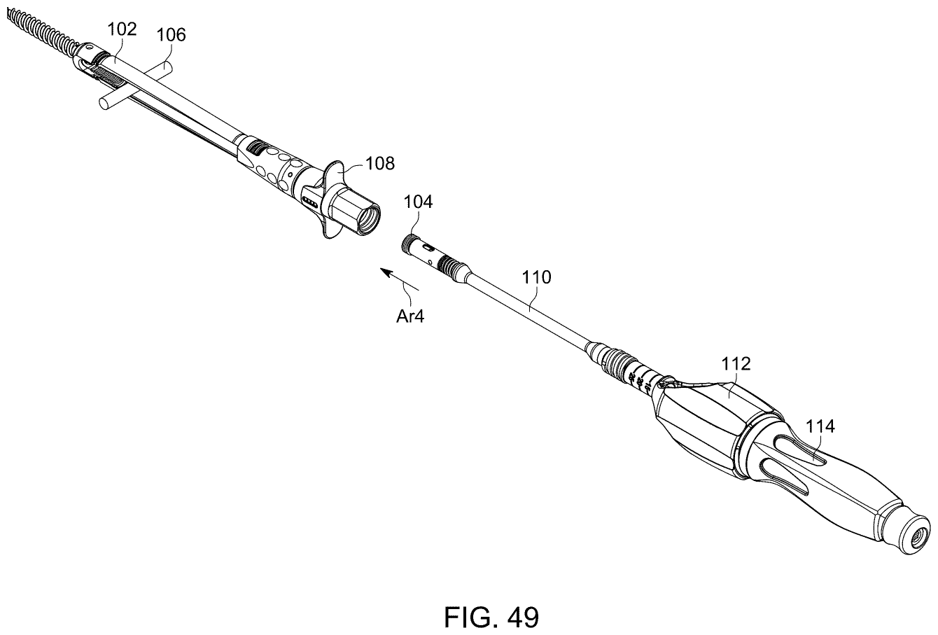

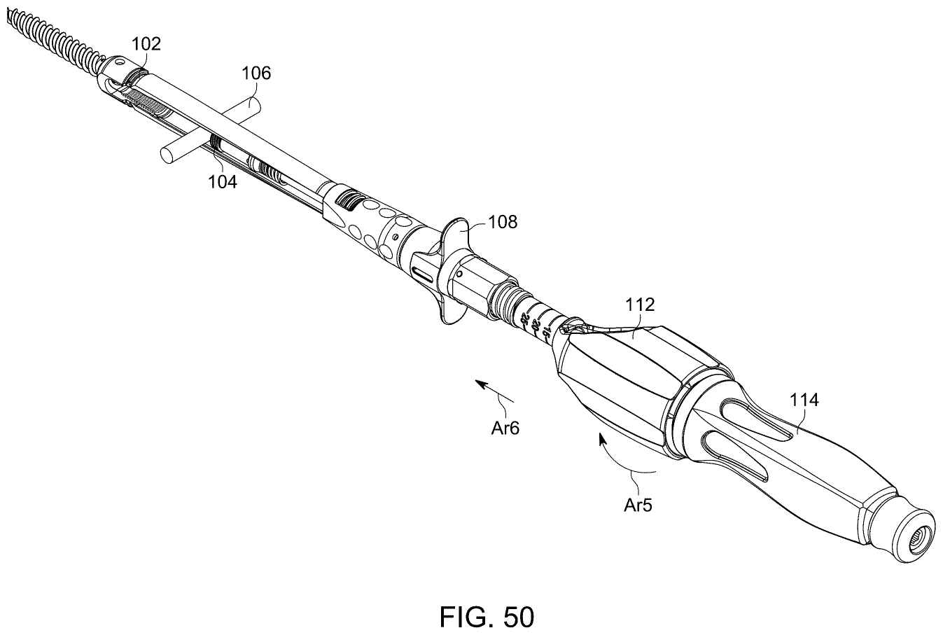

The method can include passing a distal portion of the inserter-reducer shaft 110 having the setscrew 104 disposed thereon through the lumen of the reducer clip 108 into the pedicle screw 102 until a distal threaded portion of the reducer handle 112 disposed around the shaft contacts the proximal threaded portion of the reducer clip 108. That is, the assembly from FIG. 48 can be disposed proximal to the assembly from FIG. 44, as shown in FIG. 49. The assembly from FIG. 48 can then be advanced distally, as shown by arrow Ar4 in FIG. 49, to the position shown in FIG. 50. In so doing, a distal portion of the shaft 110 with setscrew 104 disposed thereon can pass through the lumen of the reducer clip 108 into the space between the opposed extended tabs 128A, 128B of the pedicle screw 102. Further, a distal end of the reducer handle 112 can enter the lumen of the reducer clip 108 and the threads 180 formed on a distal portion of the reducer handle can contact the threads 144 formed on a proximal portion of the reducer clip. Depending on the position of the rod 106 along the length of the extended tabs 128A, 128B, a distal end of the shaft 110 extending distally beyond the setscrew 104 may or may not contact the rod 106 at this point.

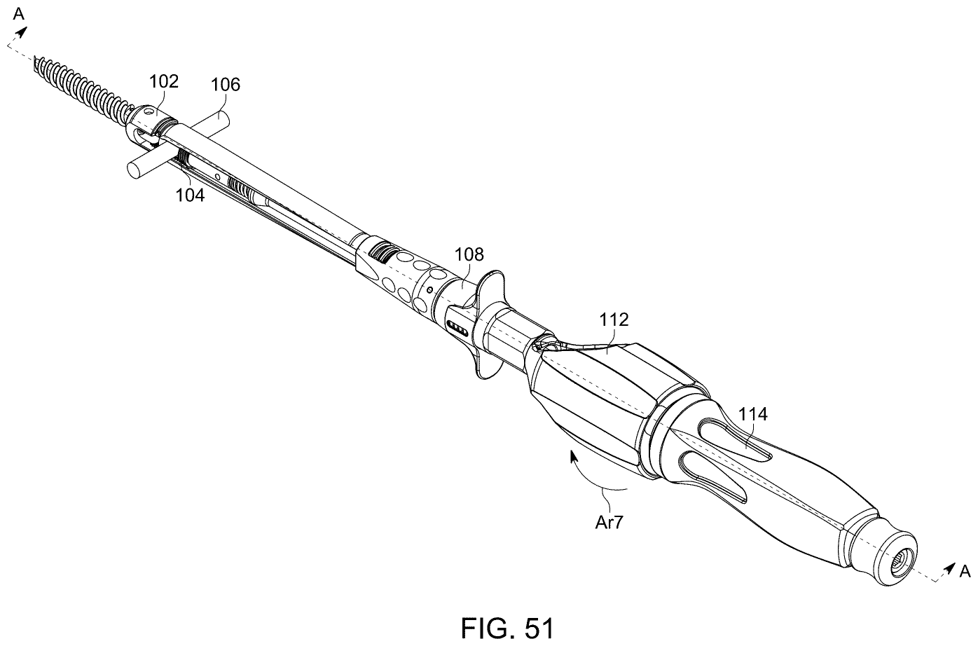

The method can include rotating the reducer handle 112 to engage the threads 180 of the reducer handle with the threads 144 of the reducer clip 108 and advance the inserter-reducer shaft 110 distally relative to the reducer clip. That is, the reducer handle 112 can be rotated, e.g., in the direction of arrow Ar5 in FIG. 50, to cause associated distal translation of the reducer handle in the direction of Ar6 in FIG. 50. During this rotation of the reducer handle 112, a distal end surface 182 thereof can bear against the thrust bearing 226 (or proximal-facing shoulder 224 if the bearing is not present) to urge the shaft 110 distally. In addition, a distal end of the shaft 110 can contact the rod 106 and urge it distally relative to the pedicle screw 102, thereby achieving reduction of the rod toward the rod seat 126. This movement can continue until the distally facing shoulder 188 of the reducer handle 112 contacts the proximal end surface 177 of the reducer clip 108, as shown in FIG. 51. At such a point, the reducer handle 112 has reduced the rod 106 to a prescribed extent and the setscrew 104 can be disposed in contact with the threads 132 of the pedicle screw 102. Further, contact between the shoulder 188 of the reducer handle 112 and the surface 177 of the reducer clip 108 can act as a hard stop to prevent overreduction of the rod 106 and setscrew 104. This can avoid potential damage from, e.g., advancing the setscrew 104 too far such that threads formed thereon interfere with threads formed on the pedicle screw 102 without proper rotational engagement therebetween.

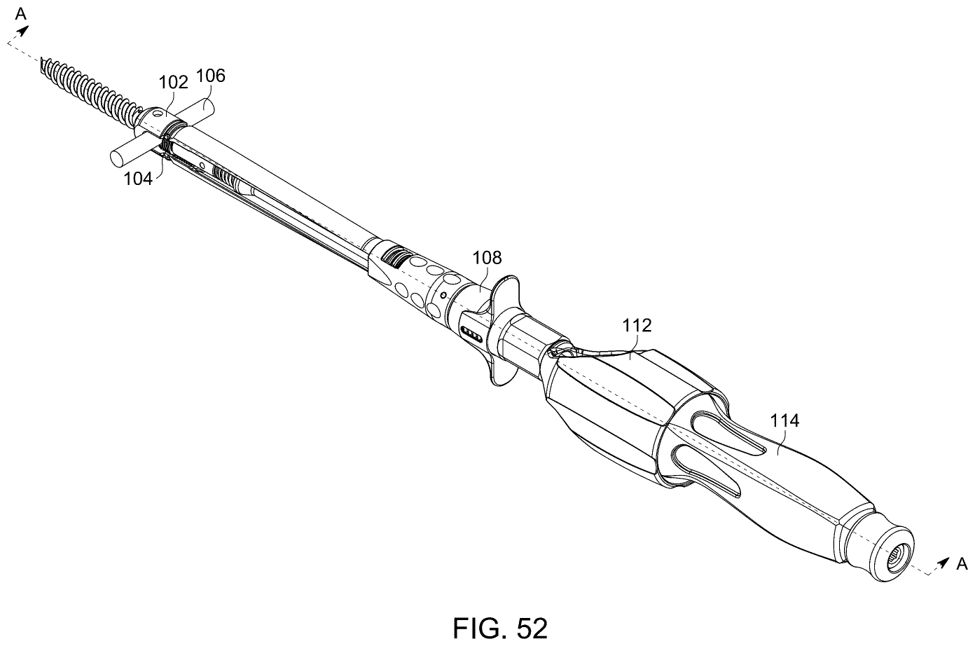

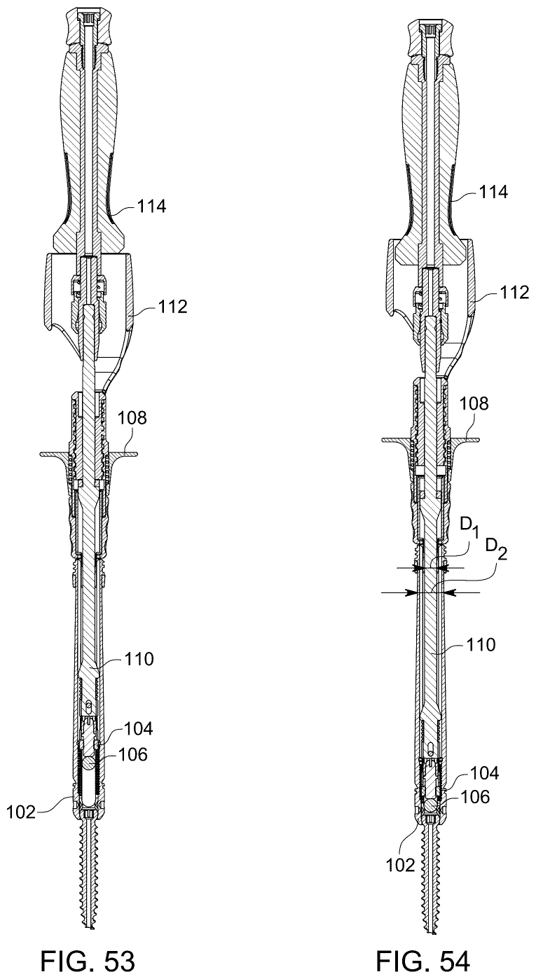

The method can include rotating the driver handle 114 coupled to the proximal end of the inserter-reducer shaft 110 (including embodiments where the driver handle and inserter-reducer shaft are modular/separable and embodiments where they are unitary or permanently coupled) extending through the reducer handle 112 to engage the threads 138 on the outer surface of the setscrew 104 with the threads 132 formed on the inner surface of the pedicle screw 102. That is, the driver handle 114 can be rotated, e.g., in the direction of arrow Ar7 in FIG. 51, to engage the threads 138 of the setscrew 104 with the threads 132 of the pedicle screw 102. Threaded engagement of the setscrew 104 and pedicle screw 102 can cause the setscrew 104 to continue advancing distally relative to the pedicle screw and also relative to the shaft 110, e.g., translating distally over the distal portion 216 on which the setscrew is disposed. This can include passing over the protrusions 242 of the spring clip 229 and beyond a distal end of the shaft 110, such that the setscrew 104 contacts the rod 106 to effect final reduction and securement of the rod within the rod seat 126 of the pedicle screw 102. Depending upon the positioning of the shaft 110 when the reducer handle 112 reaches the point of maximum reduction shown in FIG. 51, rotation of the driver handle 114 can result in some continued distal advancement of the shaft 110 and driver handle 114, as shown by the transition between FIGS. 51 and 52, as well as the counterpart cross-sectional views of FIGS. 53 and 54. This can best be visualized in these figures with reference to the relative position of the drive handle 114 relative to a proximal end of the reducer handle 112.

Certain prior reduction instruments have included the concept of multiple reduction handles (e.g., one that can be translated distally and another than can be rotated) in an instrument configured to introduce a setscrew. These instruments, however, can require a user to be careful to avoid jamming the threads of the setscrew and pedicle screw together in a way that might damage one or both of the components. For example, users can be required to carefully feel for the engagement of threads and partially rotate a driving handle, or perhaps repeatedly reposition with translation while rotating to properly engage threads due to, e.g., the translational positioning of the setscrew and the clocking of the threads. This can result in an instrument that requires great care and delicacy to operate correctly.

The reduction instruments disclosed herein improve on such prior instruments at least by providing the distal portion with spring-loaded sleeve 218 or other component or structure (e.g., a compressible elastomer, etc.) that distally biases the setscrew 104 along a distal portion of the inserter-reducer shaft 110. This can allow a user to bottom out the reducer handle 112 relative to the reducer clip 108 and then simply begin rotating the driver handle 114 without concern for delicately adjusting the instrument to properly engage the setscrew threads. As noted, this is made possible by the extended distal portion 216 of the shaft 110, along which the setscrew 104 is disposed and can translate, and which extends distally from the setscrew 104 to contact the rod 106, as well as the extended-length spring clip 229 that selectively retains the setscrew on the distal portion of the shaft and the distally-biased sleeve 218 that urges the setscrew distally while allowing proximal movement with sufficient force to overcome the spring 220.

As a result, the reduction instruments disclosed herein can utilize a single inserter-reducer shaft 110 to push distally on the spinal rod 106, as well as deploy the setscrew 104. The improved inserter tip design can allow for streamlined setscrew deployment relative to prior instruments, however. For example, the inserter tip can have a lengthened X25 drive feature (i.e., distal portion 216) with the spring-loaded sleeve 218 that biases the setscrew 104 distally while still allowing the setscrew to translate proximally. During use, the reducer handle 112 can be rotated to advance the inserter-reducer shaft 110 and reduce the spinal rod 106. When the shoulder 188 on the reducer handle 112 bottoms out on the proximal end 177 of the reducer clip 108, the setscrew threads 138 can be in contact with the female threads 132 on the pedicle screw extended tabs 128A, 128B, and the spring-loaded sleeve 218 can be pushing the setscrew distally. This hard stop for reduction using the reducer handle 112 can position the setscrew 104 in contact with the threads 132 on the pedicle screw without overdriving the two components in a manner that may damage them. The driver handle 114 can be turned to rotate the inserter-reducer shaft 110 and thread the setscrew 104 into the pedicle screw receiver head 118. Unlike certain prior instruments, a user does not have to worry about driving the setscrew 104 too far into the pedicle screw receiver member 118 or repeatedly go back-and-forth between the reducer and driver handles 112, 114 to get the setscrew threads 138 to start to engage with the pedicle screw threads 132. Once the setscrew threads 138 are engaged, the driver handle 114 can continue to be rotated to thread the setscrew distally along the reduction threads 132 on the pedicle screw 102. The proximal portion of the inserter-reducer shaft 110 can be long enough to allow the shaft to continue to travel distally with the setscrew while avoiding any interference between components, e.g., the reducer handle 112 and driver handle 114, etc.

FIGS. 55-57 illustrate the operation of the improved distal portion of the inserter-reducer shaft 110 described above. In the view of FIG. 55, the rod 106 can be in contact with a distal end of the shaft 110 and the shaft can be advancing distally as a result of rotation of the reducer handle 112. In this view, the setscrew 104 and threads 138 thereof can make initial contact with the threads 132 on the pedicle screw. Also, the sleeve 218 can be in contact with the setscrew and disposed at a distally advanced position, e.g., urging the setscrew 104 against the protrusions 242 of the spring clip 229 that retain the setscrew relative to the shaft 110 (prior to such a position, the setscrew may or may not be in contact with the sleeve).

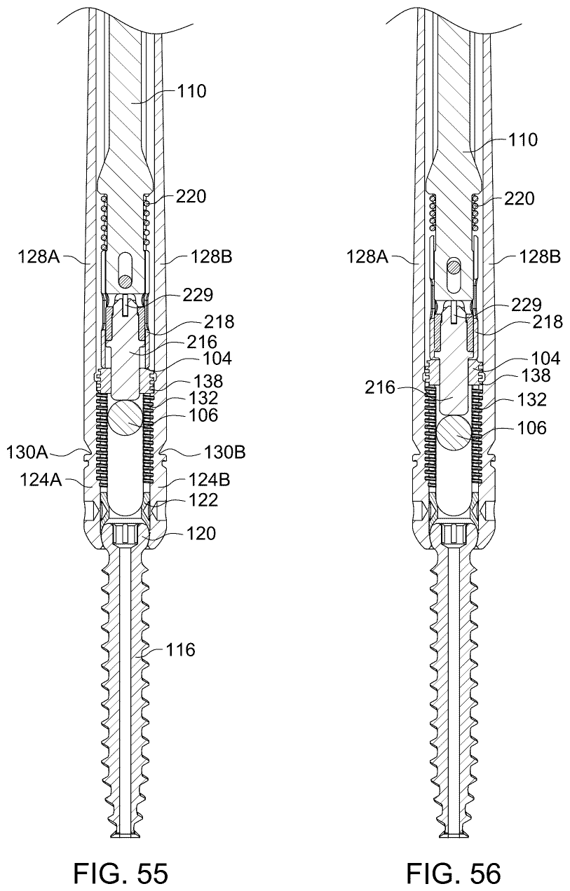

As a user continues to rotate the reducer handle 112 and bottom it out on the reducer clip 108, the instrument can transition from the view of FIG. 55 to the view of FIG. 56. In this configuration, the rod 106 has been reduced farther relative to the pedicle screw 102 (e.g., compare its position in FIGS. 55 and 56 relative to the threads 132) by the shaft 110. The setscrew 104, however, has not moved and remains in contact with a proximal end of the threads 132 of the pedicle screw 102. This means the setscrew 104 has translated proximally along the distal portion 216 of the shaft 110 against the biasing force of the spring 220. The sleeve 218 that is in contact with the setscrew 104 has also moved proximally relative to the shaft 110 and the spring 220 is in a more compressed state.

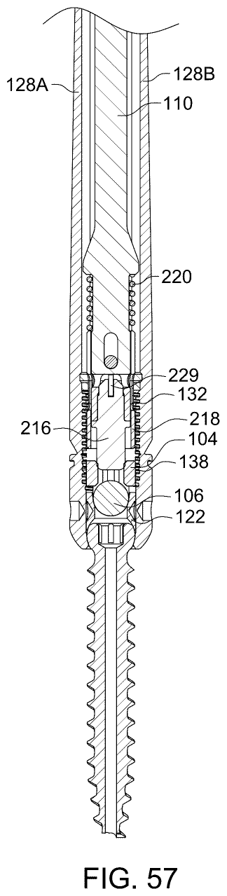

A user can further reduce the rod and secure its position by rotating the driver handle 114. The user can begin rotating the driver handle immediately after bottoming out the reducer handle 112 against the reducer clip 108 because the setscrew 104 has been maintained in an ideal position at the proximal end of the threads 132 by the spring-loaded sleeve 218. No undesired distal force has been applied to the setscrew 104 during the reduction operation that could potentially damage the threads of the setscrew or pedicle screw 102. Rotation of the driver handle 114 can result in a transition from the configuration of FIG. 56 to the configuration of FIG. 57. In this view, the setscrew 104 is translating distally off the shaft 110 as it is rotated by the shaft and driver handle 114, which can include deforming the spring clip 229 radially inward to allow passage of the setscrew over the protrusions 242 of the spring clip. The sleeve 218 has distally advanced to the limit of its range of motion with corresponding relaxing of the spring 220 and the setscrew 104 has separated from the sleeve 218 as it advances distally along the threads 132 of the pedicle screw 102. In this configuration, a user can perform final tightening of the setscrew 104 relative to the pedicle screw 102, thereby securely capturing the rod 106 to the pedicle screw. In some embodiments, however, final tightening can be performed at a later point in a procedure.

Regardless of final tightening, once the setscrew 104 is engaged with the threads 132 of the pedicle screw to a desired degree, the instrument 100 can be separated from the pedicle screw 102, rod 106, and setscrew 104. The instrument 100 can be separated from the pedicle screw 102 as a single unit in some embodiments, or it can be disassembled in a component-by-component fashion. To separate as a single unit, a user can proximally withdraw the locking sleeve 150 of the reducer clip 108 to retract the locking tabs 164 from the space between the extended tabs 128A, 128B. While holding the locking sleeve 150 in the proximally withdrawn position, the extended tabs 128A, 128B can be squeezed or moved radially inward toward one another to pass the threads 134 and/or flange 135 thereof out of the recesses 142A, 142B of the reducer clip 108, and the instrument 100 can be withdrawn proximally relative to the pedicle screw 102. The diameter D₁ (see FIGS. 39, 40, and 54) of the inserter-reducer shaft 110 in the area of the reducer clip 108 can be smaller than a distance D2 (see FIGS. 39, 40, and 54) between the extended tabs 128A, 128B to allow the extended tabs 128A, 128B to be squeezed together in order to remove all parts of the reducer instrument 100 together.

As noted above, the instrument 100 described above can provide a novel solution to extending the reduction capability of an extended tab pedicle screw. For example, in one embodiment a pedicle screw having extended tabs can provide for about 10 mm of rod reduction, e.g., using the threads 132 formed along an inner surface of the receiver member 118. The reducer clip 108 disclosed herein can extend the available reduction capacity. In one embodiment, for example, the reducer clip can provide an additional about 30 mm of reduction along threads 144, resulting in a total reduction capacity of about 40 mm. The reducer clip disclosed herein can also be useful by providing additional length extending proximally from the pedicle screw. For example, in one embodiment the pedicle screw extended tabs can have a length of about 100 mm. In certain larger patients, however, this can be shorter than desired, e.g., to reach from a vertebra above a skin surface, etc. In such a case, the added length of the reducer clip 108 can be helpful during a procedure to facilitate grasping and/or manipulating the pedicle screw 102 or other components, e.g., when targeting insertion of a setscrew or other component, etc.

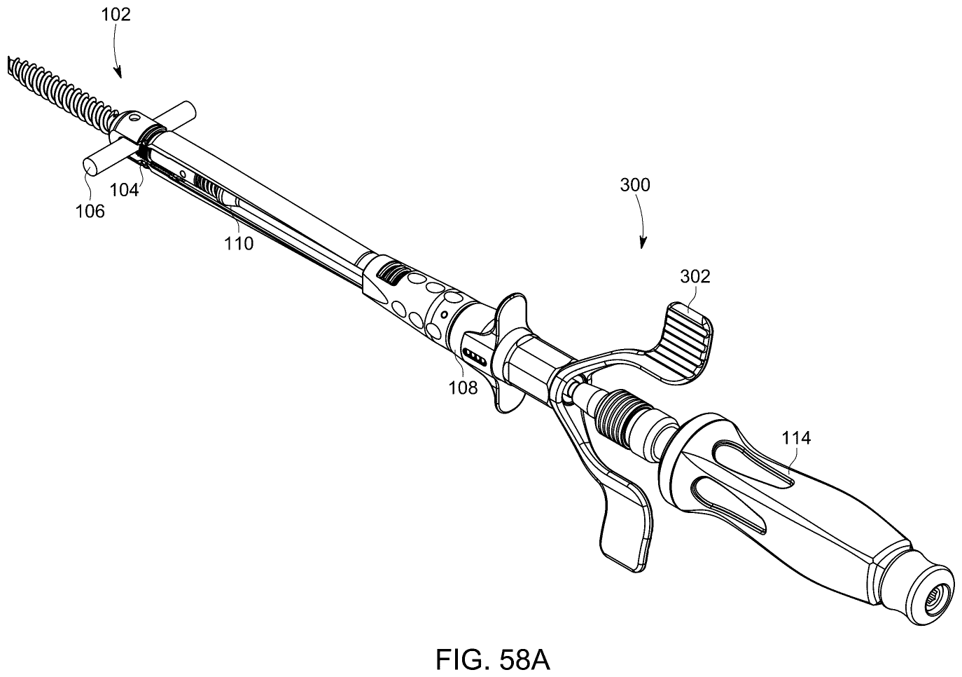

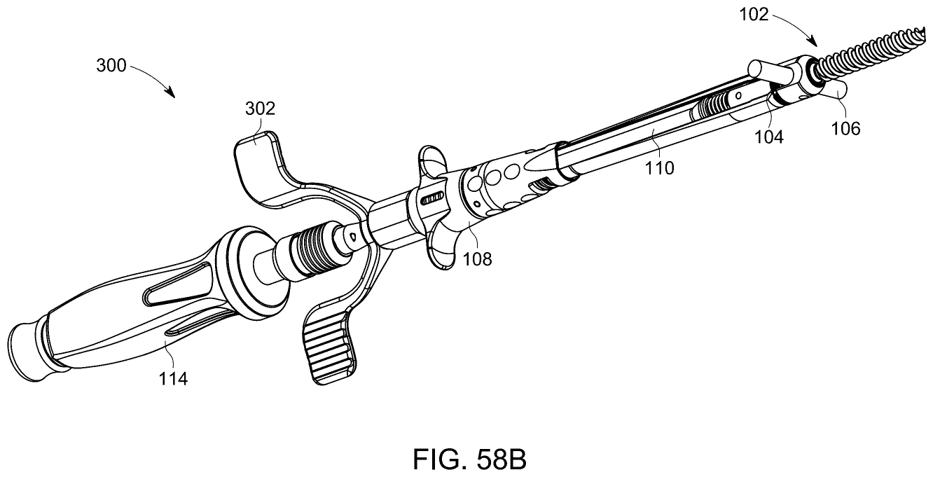

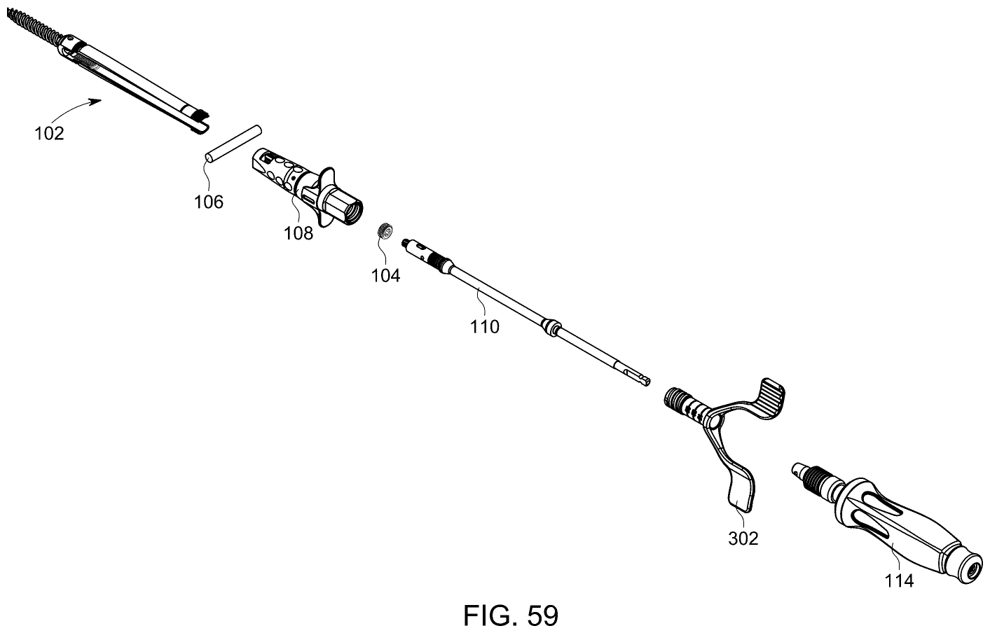

FIGS. 58A-61 illustrate another embodiment of an assembly including a pedicle screw reducer instrument 300 according to the present disclosure. The instrument 300 can be similar to the instrument 100 described above and a detailed description of every component is therefore omitted for brevity. For example, the instrument 300 can include the same reducer clip 108, inserter-reducer shaft 110, and driver handle 114 as the instrument 100 described above. In addition, the instrument 300 can be utilized in connection with the same pedicle screw 102, setscrew 104, and spinal fixation rod 106 as the instrument 100 described above.

The instrument 300 can include an alternative reducer handle 302 that has a different form factor from the reducer handle 112 described above. In particular, the reducer handle 302 can have a wing-nut-like handle with opposed wings or paddles 304A, 304B configured to interface with a user and facilitate application of torque to the reducer handle to effect distal reduction or translation of the rod 106 relative to the pedicle screw 102.

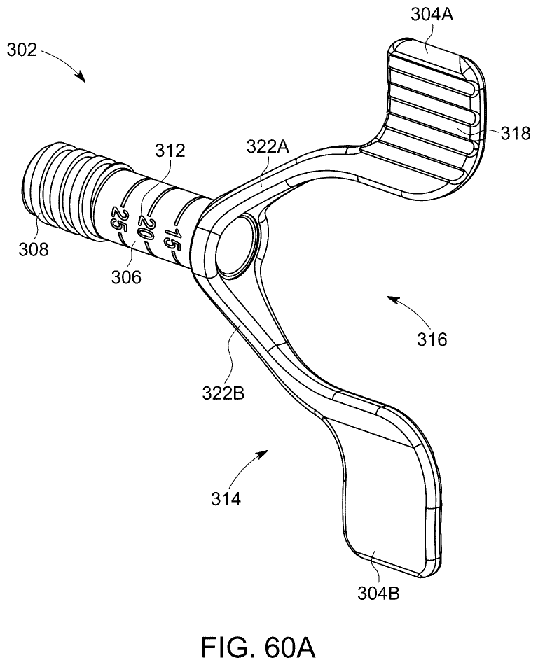

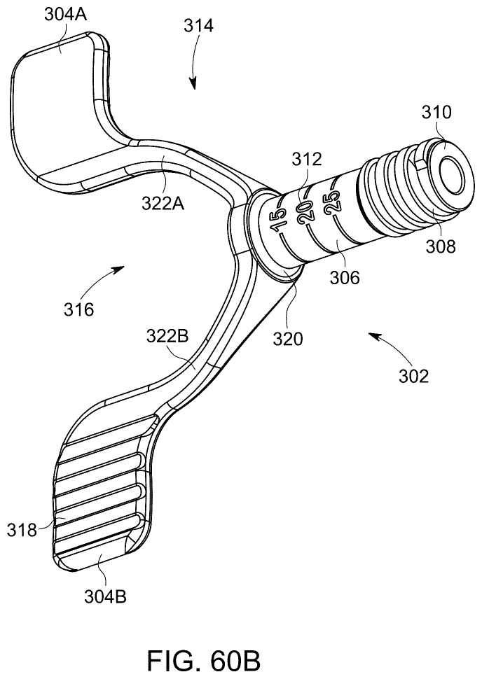

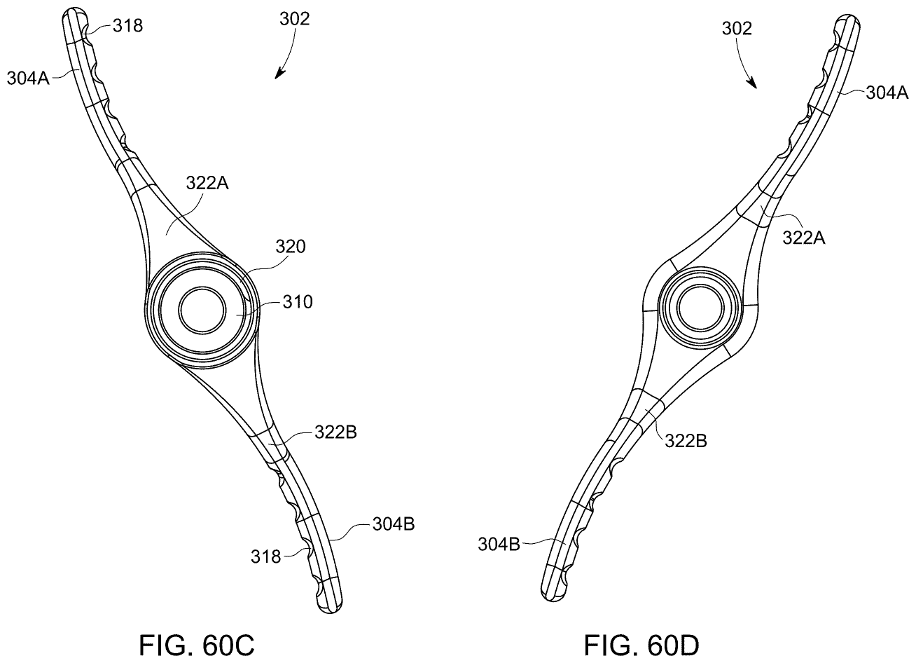

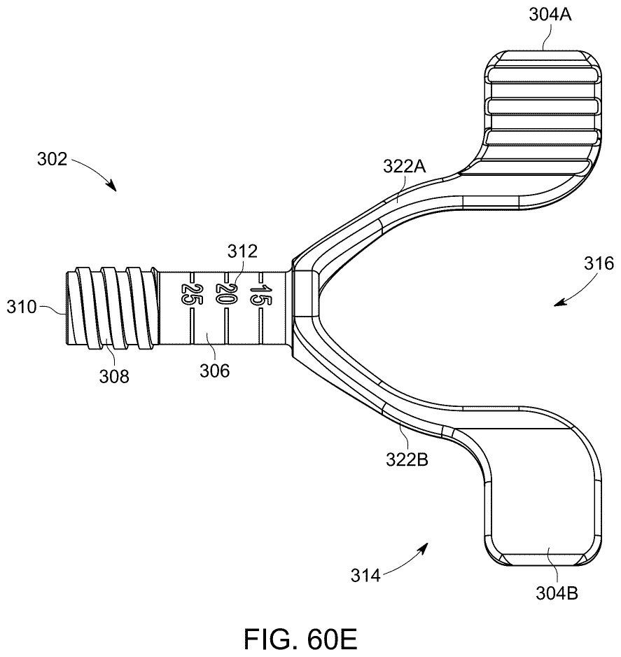

As shown in the detail views of FIGS. 60A-60E, a distal portion of the reducer handle 302 can be similar to the reducer handle 112 described above. That is, it can include a distal portion 306 having a generally cylindrical profile with a lumen extending therethrough, e.g., to accommodate passage of the inserter-reducer shaft 110 through the reducer handle 302. The distal portion 306 can have a size configured to be received within the lumen of the reducer clip 108. In addition, the distal portion 306 can include threads 308 formed on an external surface thereof that are configured to interface with the threads 144 formed on an inner surface of the proximal sleeve 148 of the reducer clip 108 to provide for controlled movement of the reducer handle 302 relative to the reducer clip, which can thereby provide for control of reduction of a spinal rod 106 and/or setscrew 104 relative to a pedicle screw 102 coupled to the reducer clip 108.

The distal portion 306 of the reducer handle 302 can also include a blunt distal end surface 310 configured to contact the inserter-reducer shaft 110 in order to impart a distally-directed force thereto during reduction. The blunt distal end surface 310 can be substantially smooth and flat such that it is configured to bear against another flat surface on the shaft or a thrust washer or bearing disposed therebetween, to facilitate imparting an axial translation force to the shaft while allowing for relative rotation between the shaft and the reducer handle.

Still further, the distal portion 306 of the reducer handle 302 can include one or more markings 312 formed on an external surface thereof to provide feedback to a user regarding a reduction distance or amount indicated by the relative positioning of the reducer handle and the reducer clip 108. While different amounts of reduction are possible by varying the lengths of the reducer clip 108 and reducer handle 302, in the illustrated embodiment the reducer clip and reducer handle are configured to provide up to about 30 mm of rod reduction. Different marking or other feedback schemes are possible but, in the illustrated embodiment, an outer surface of the distal portion 306 of the reducer handle 302 includes markings 312 to show a distance of reduction that remains at a point where a proximal end of the reducer clip 108 aligns with a particular distance marking.

The reducer handle 302 can also include a proximal portion 314 configured for a user to grasp and apply torque thereto when performing rod reduction. The proximal portion 314 can include the opposed wings or paddles 304A, 304B separated by a space 316 that can receive, e.g., a proximal portion of the inserter-reducer shaft 110 and a distal portion of the reducer handle 114. An external surface of each wing can include one or more features formed thereon, such as grooves 318 to aid a user in gripping and/or grasping and/or applying torque to the reducer handle 302. Any of a variety (and number) of surface features can be utilized (e.g., texturing, knurling, finger-shaped recesses, etc.).

The distal portion 306 and the proximal portion 314 can be separated by distally-facing step or shoulder 320 that can serve as a stop or limit on rod reduction (at least, on rod reduction provided by the reducer handle 302 and inserter-reducer shaft 110 versus, for example, what might be additionally possible using the setscrew 104 when driven by the driver handle 114). For example, the shoulder 320 can contact a proximal end of the reducer clip 108 at a point of maximum rod reduction, thereby preventing any further reduction using the reducer handle 302. Contact between the shoulder 320 and the reducer clip 108 can serve as a hard stop to prevent overreduction that might, e.g., drive the setscrew 104 into the threads 132 of the pedicle screw 102 with too much force. The wings 304A, 304B can extend radially outward from a central longitudinal axis of the instrument by a greater distance than a diameter of the distal portion 306. To accommodate the different diameters, a distal portion of the proximal portion 314 can include connecting arms 322A, 322B transitioning therebetween.

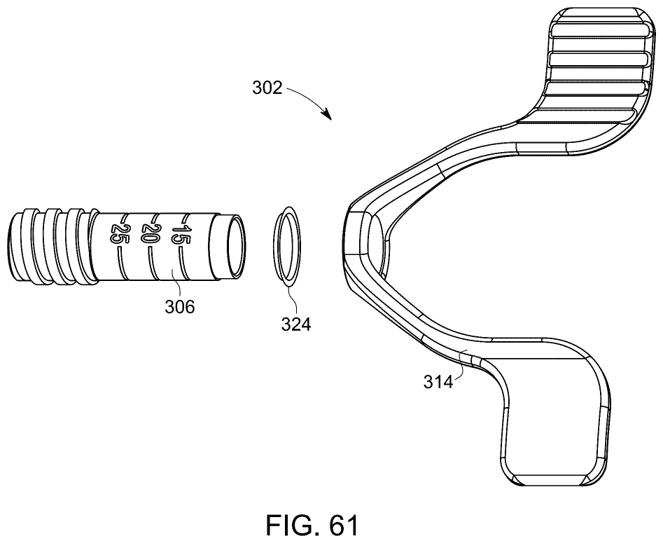

In some embodiments, the reducer handle 302 can be formed from a unitary or monolithic piece of material, e.g., through a single injection molding or other manufacturing operation. In certain embodiments, however, the reducer handle 302 can be formed from a plurality of components coupled. For example, FIG. 61 shows an exploded view of an embodiment wherein the reducer handle distal portion 306 and proximal portion 314 are separate components. In this embodiment, the reducer handle 302 can also include a ring 324 that can be disposed therebetween. These components can be coupled in manner that allows for selective disassembly when desired, e.g., threading, interference fit, etc., or can be joined in a more permanent manner such that disassembly is not contemplated after manufacturing, e.g., using an adhesive, welding, etc.

As noted above, operation of the instrument 300, including the reducer handle 302, is substantially the same as with instrument 100 described above. Accordingly, a detailed description of the operation and methods of use of the instrument 300 is omitted here for brevity.

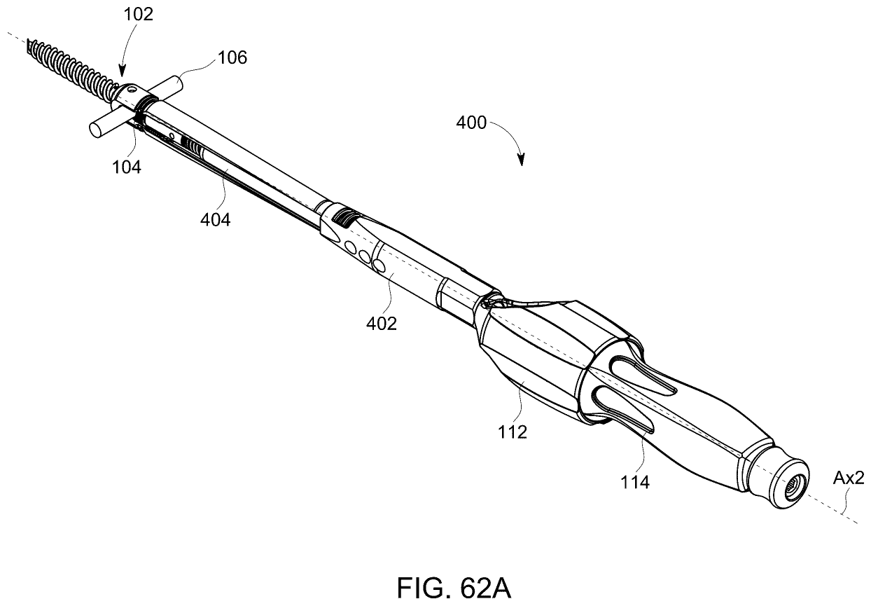

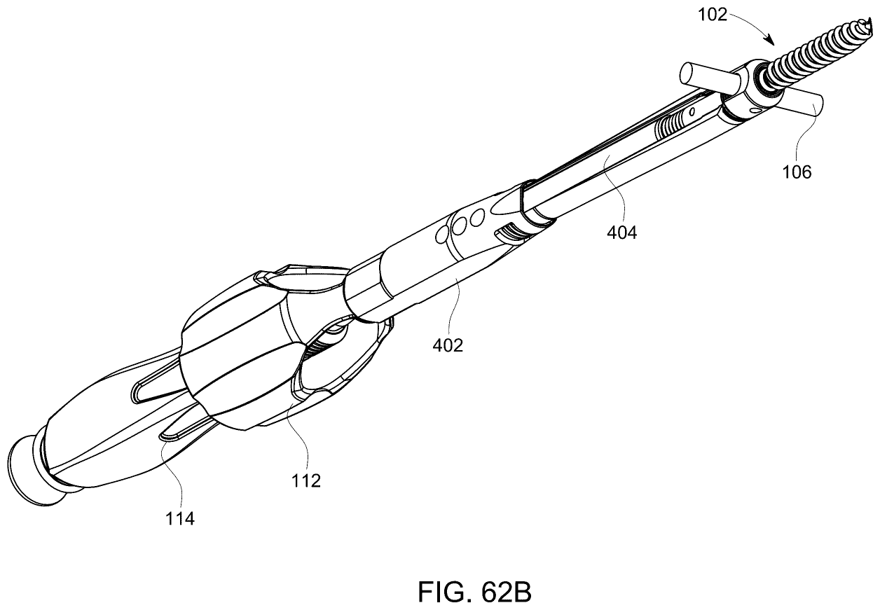

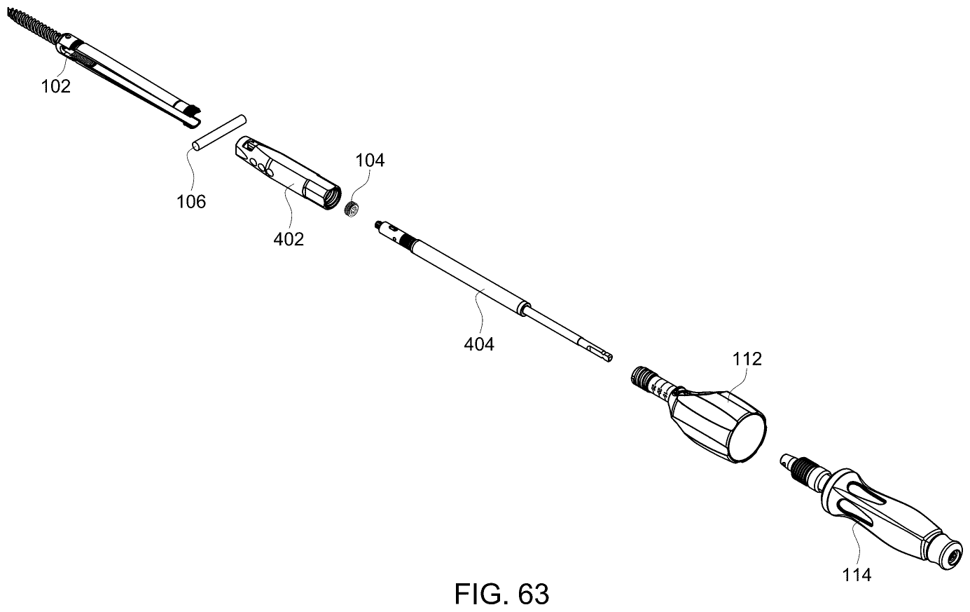

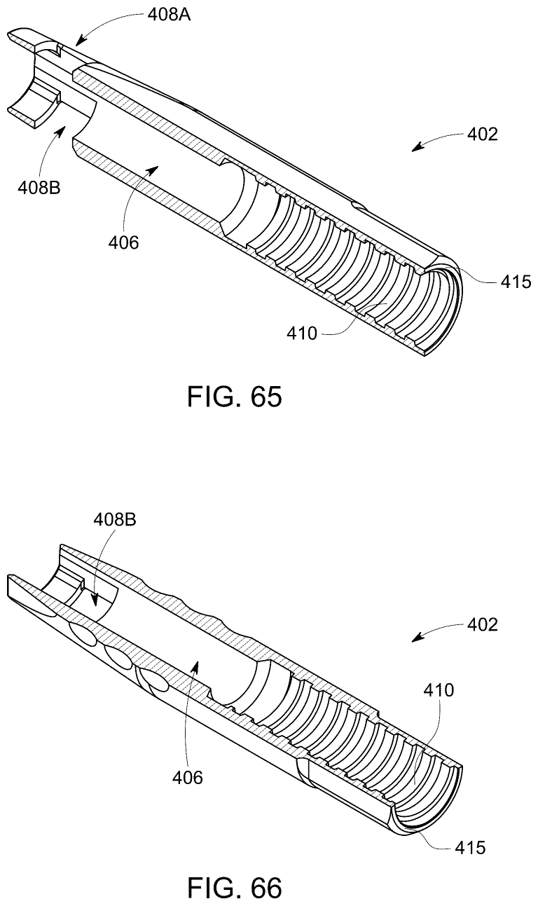

FIGS. 62A-79 illustrate another embodiment of an assembly including a pedicle screw reducer instrument 400 according to the present disclosure. The instrument 400 can be similar to the instrument 100 described above and a detailed description of every component is therefore omitted for brevity. For example, the instrument 400 can include the same reducer handle 112 and driver handle 114 as the instrument 100 described above. In addition, the instrument 400 can be utilized in connection with the same pedicle screw 102, setscrew 104, and spinal fixation rod 106 as the instrument 100 described above.

The instrument 400, however, can include an alternative embodiment of a reducer clip 402 that does not utilize a spring-loaded locking sleeve to facilitate coupling to a pedicle screw 102. Instead, as explained in more detail below, the instrument 400 utilizes a unitary reducer clip 402 and an inserter-reducer shaft 404 with an enlarged diameter along a central portion thereof that can prevent radially-inward movement of the extended tabs 128A, 128B of the pedicle screw 102 and thereby avoid unintended separation of the reducer clip from the pedicle screw.

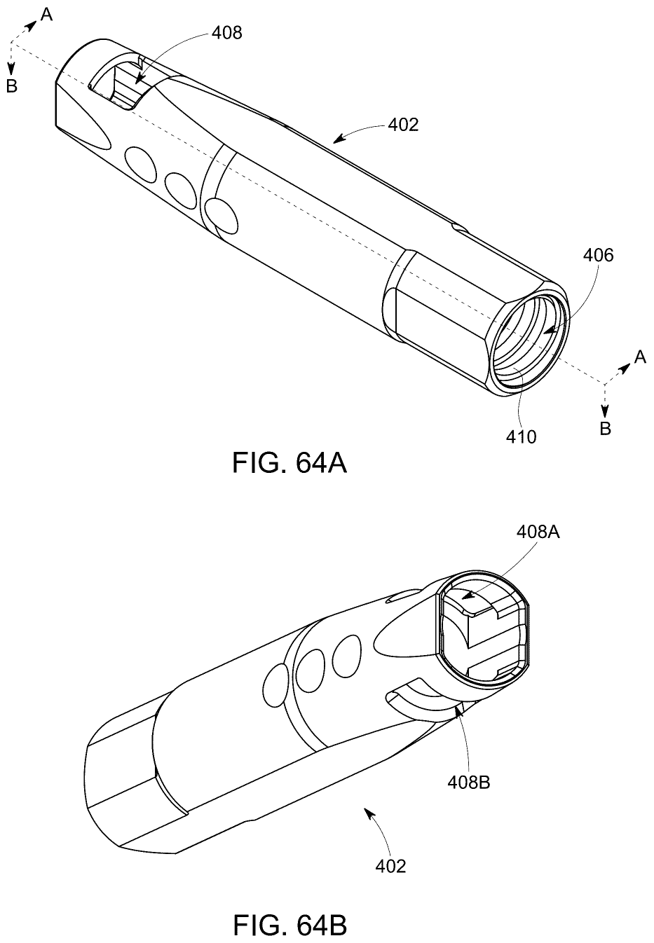

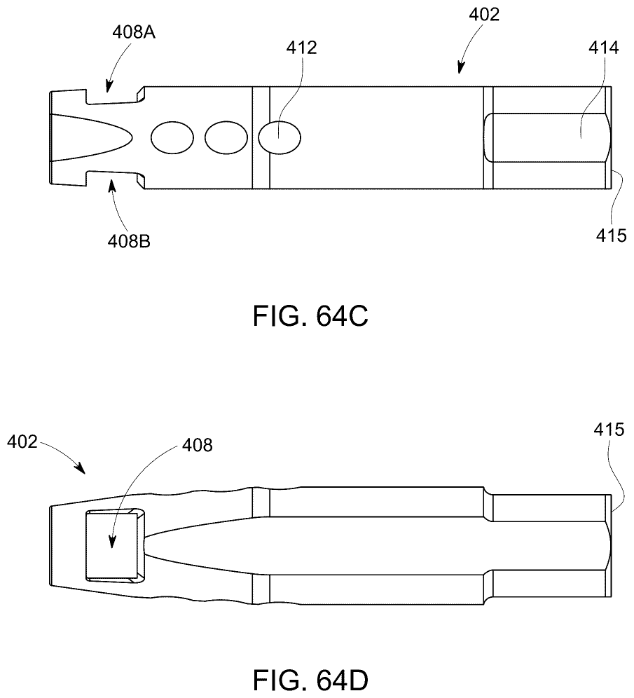

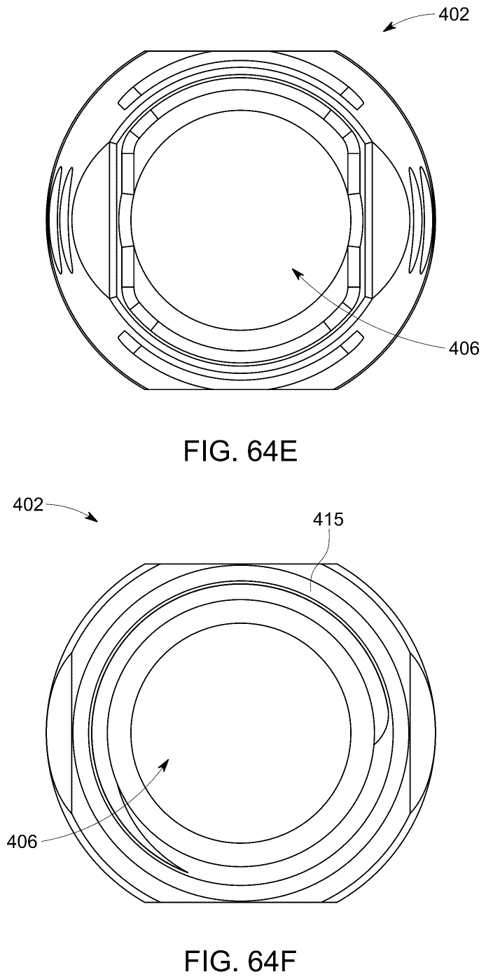

FIGS. 64A-66 illustrate the reducer clip 402 in isolation. The reducer clip 402 can be envisioned as a single-piece combination of the above-described distal sleeve 146 and proximal sleeve 148 of the reducer clip 108. For example, the reducer clip 402 includes a lumen 406 extending therethrough and a distal portion of the reducer clip includes recesses 408A, 408B formed in a sidewall thereof that can be configured to receive the threads 134 and/or flange 135 formed on an external surface of each extended tab 128A, 128B. Further, a proximal portion of the reducer clip 402 can include threads 410 configured to interface with the reducer handle 112. An external surface of the reducer clip 402 can include one or more features to facilitate grasping or manipulating the reducer clip and/or other components coupled thereto. Any of a variety (and number) of surface features can be utilized, including features 412 that facilitate a user directly grasping the reducer clip 402 (e.g., texturing, knurling, finger-shaped recesses, etc.), as well as features 414 that facilitate doing so using an intermediate tool (e.g., flat surfaces for applying a wrench or other tool, etc.).

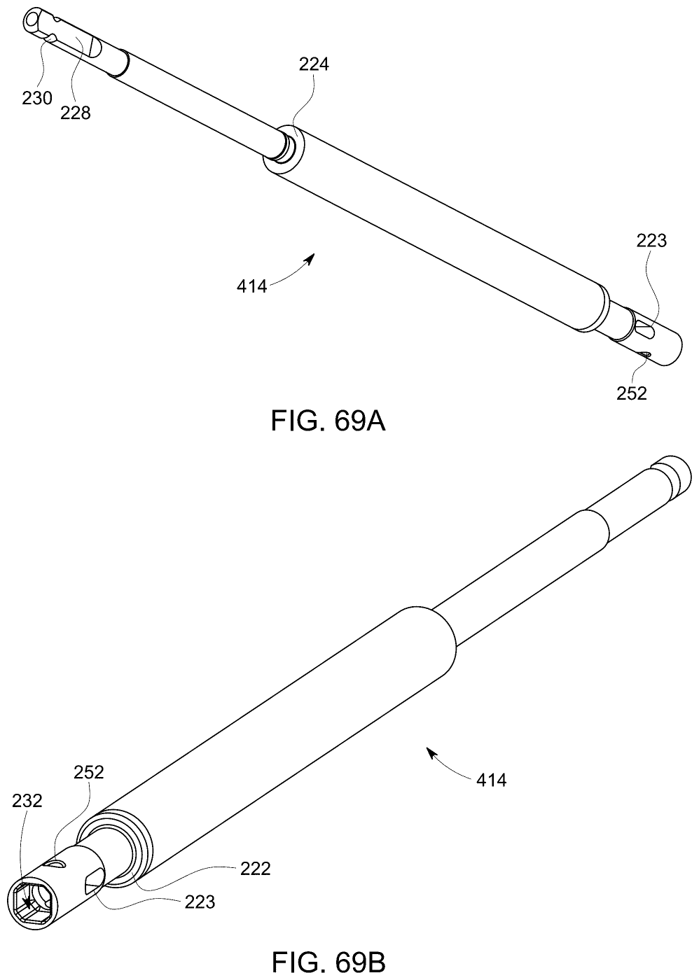

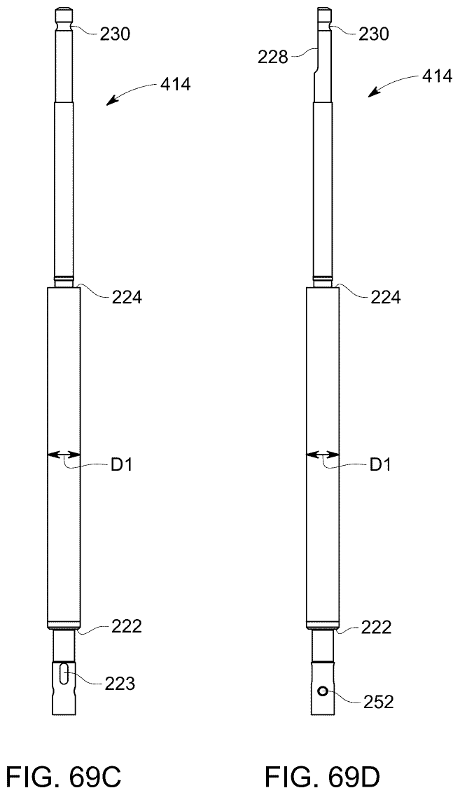

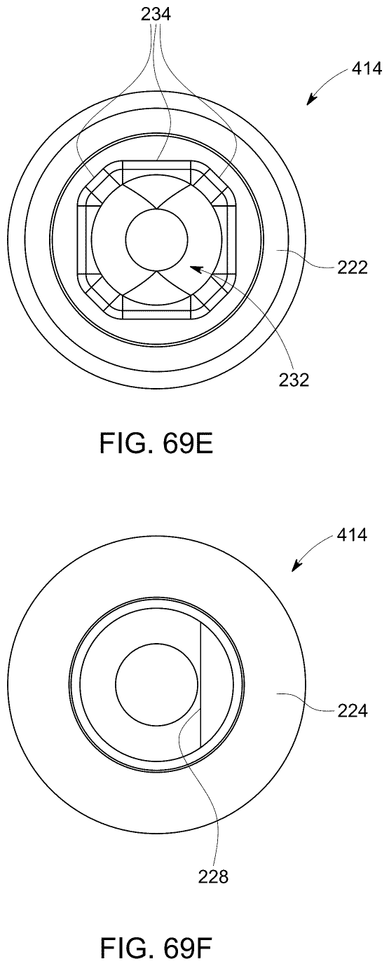

FIGS. 67A-67F illustrate the inserter-reducer shaft 404 in isolation. The inserter-reducer shaft 404 can be largely similar to the inserter-reducer shaft 110 described above, including utilizing the same distal end components that provide a spring-loaded sleeve 218 to bias a setscrew distally during use. The primary difference relative to the inserter-reducer shaft 110 is the different shape of the elongate body 414, shown in isolation in FIGS. 69A-69F.

The elongate body 414 can have similar proximal and distal end geometries, including the use of one or more flats 228 and one or more at least partial circumferential grooves 230 along a proximal portion thereof, as well as a distal-facing recess 232 with one or more flats 234, along with an elongate slot 223 and through-hole 252 along a distal portion thereof. The elongate body 414 can also include a distal-facing shoulder 222 along a distal portion thereof, and a proximal-facing shoulder 224 along a proximal portion thereof.

An intermediate portion of the elongate body 414 extending between the two shoulders 222, 224, however, can have an enlarged diameter D₁ relative to the elongate body 215 of the inserter-reducer shaft 110 described above. The enlarged diameter D₁ can be, for example, similar to the diameter of the shoulders 222, 224, such that the intermediate portion of the elongate body 414 extending between the two shoulders can have a constant diameter in some embodiments. The diameter D₁ can be selected to match the nominal inner diameter between the opposed extended tabs 128A, 128B in a resting position, as explained in more detail below.

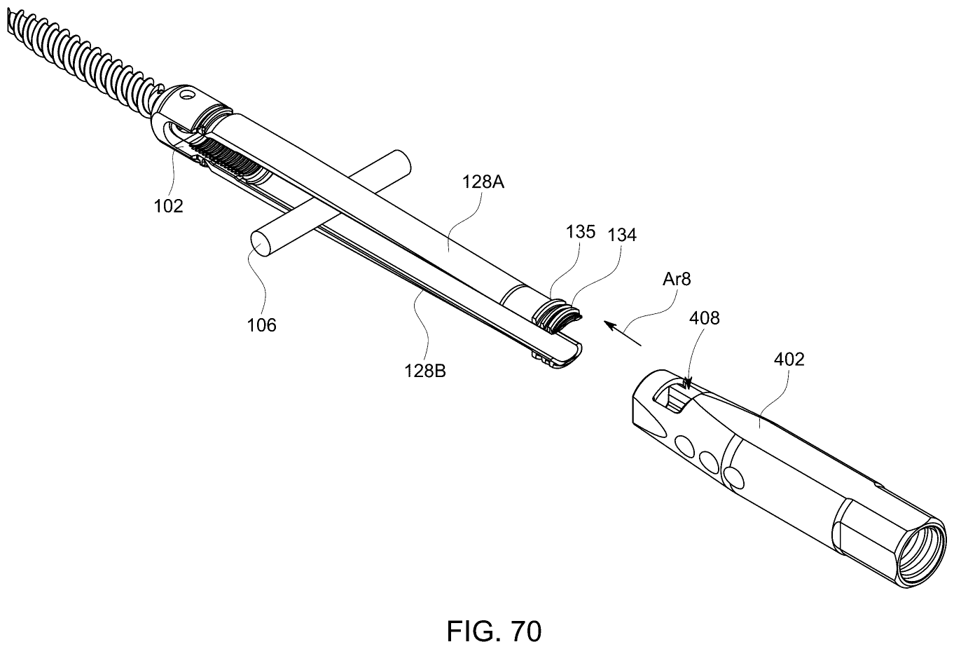

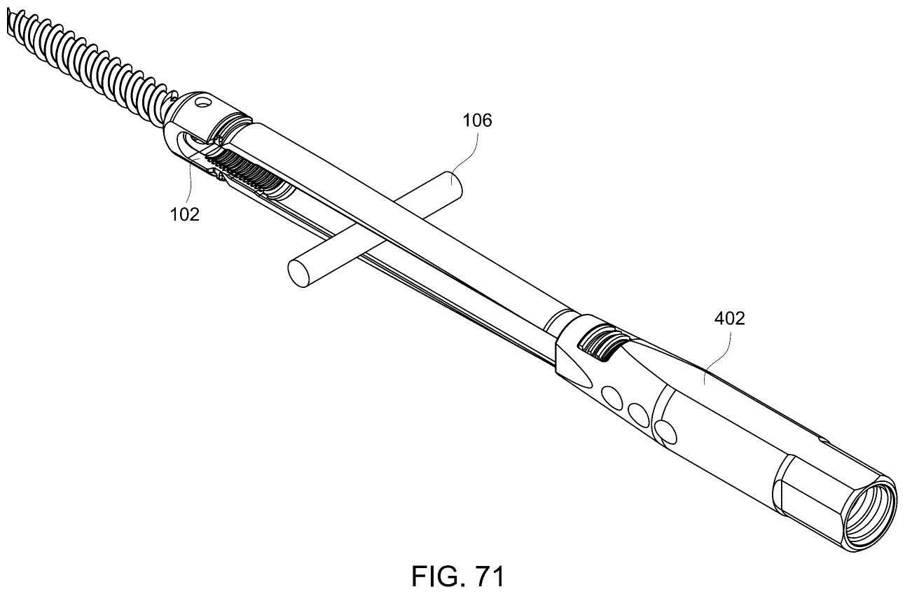

FIGS. 70-79 illustrate one example method of utilizing the instrument 400. As shown in FIG. 70, a pedicle screw 102 can be aligned with a spinal fixation rod 106 such that the rod is disposed between the opposed extended tabs 128A, 128B of the pedicle screw. The reducer clip 402 can be brought near a proximal end of the pedicle screw 102. The reducer clip 402 can be moved distally, as shown by arrow Ar8 in FIG. 70, from the position of FIG. 70 to the position of FIG. 71. In connection with advancing the reducer clip 402 distally, the extended tabs 128A, 128B of the pedicle screw 102 can be squeezed together or moved radially inward toward a longitudinal axis Ax2 (see FIG. 62A) to permit the proximal ends of the extended tabs to pass into the lumen of the reducer clip 402.

Once the threads 134 and/or flange 135 are aligned with the recesses 408 formed in the reducer clip 402, the extended tabs 128A, 128B can be allowed to return radially outward to their normal resting position. In doing so, the threads 134 and/or flange 135 can enter the recesses 408 to couple the reducer clip 402 to the pedicle screw 102, as shown in FIG. 71.

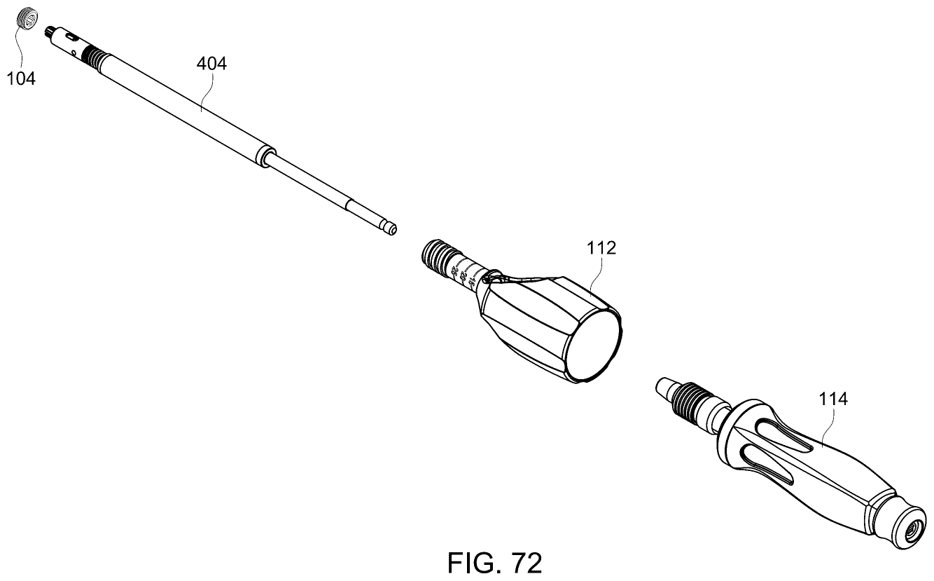

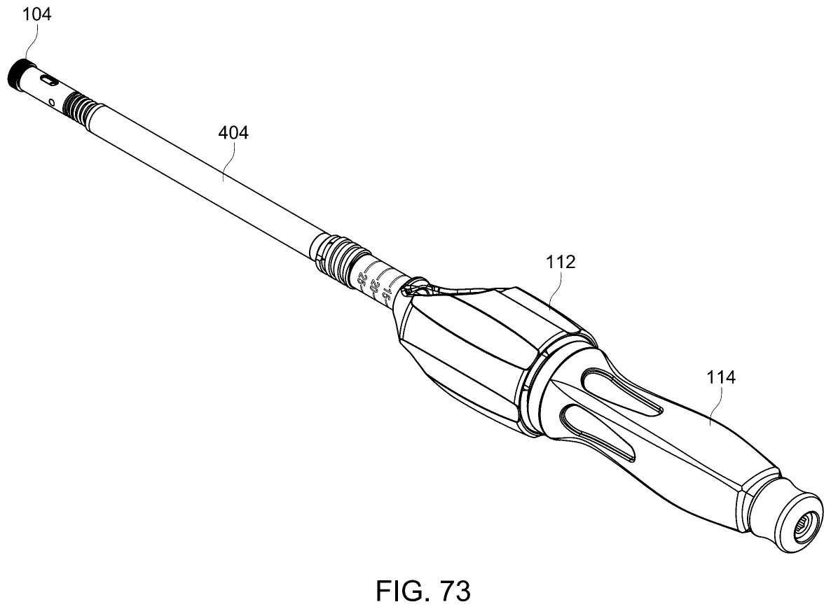

As shown in FIG. 72, the various other components of the instrument 400 can be assembled and readied for use with the reducer clip 402. For example, a setscrew 104 can be loaded onto the distal portion 216 of the inserter-reducer shaft 404. In addition, a proximal end of the inserter-reducer shaft 404 can be passed proximally through the lumen of the reducer handle 112 and passed into the distal-facing recess of the driver handle 114. FIG. 73 shows the components of FIG. 72 assembled together for use.

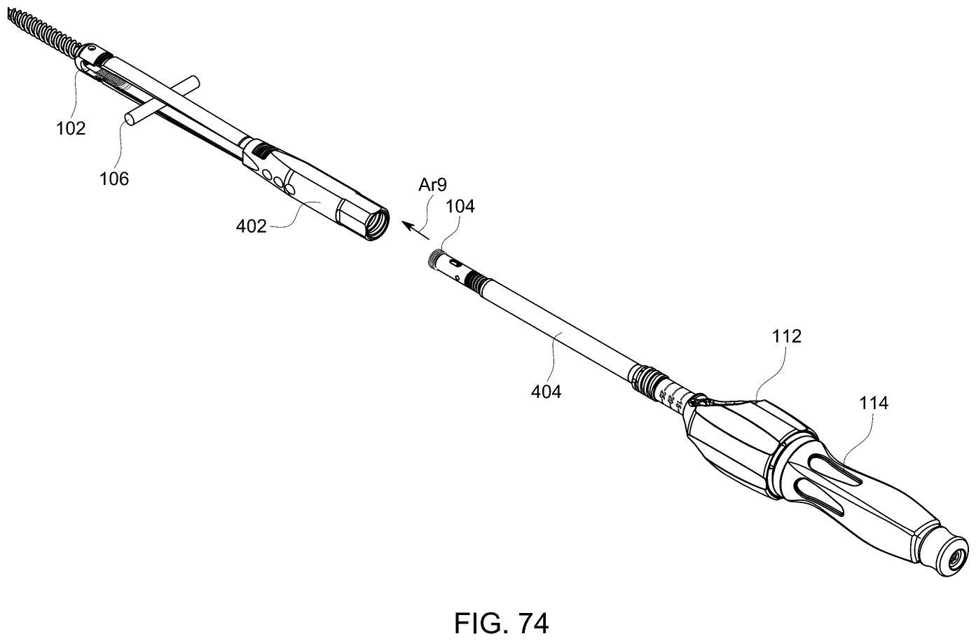

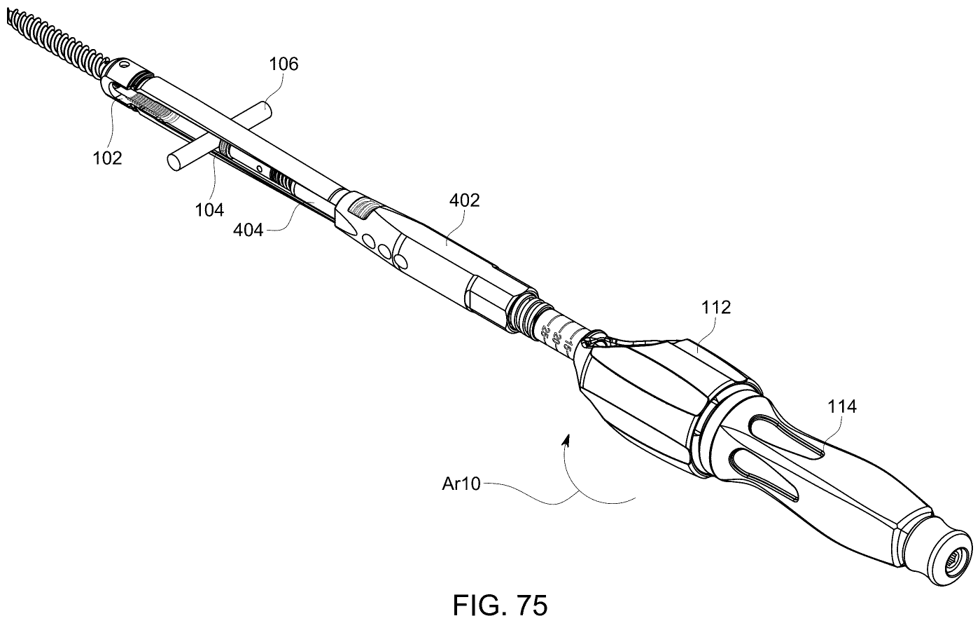

As shown in FIG. 74, the assembled components of FIG. 73 can be disposed near a proximal end of the reducer clip 402 and advanced distally, as shown by arrow Ar9 to the position shown in FIG. 75 when the threads 180 of the reducer handle 112 contact the threads 410 of the reducer clip 402. Note that, in this configuration, the intermediate portion of the inserter-reducer shaft 404 can be disposed through the lumen of the reducer clip 402, especially over a distal portion thereof where the recesses 408 are disposed. As noted above, the outer diameter D₁ of the shaft 404 in this area can substantially match the inner diameter or distance between the opposed tabs 128A, 128B. As a result, the presence of the shaft 404 can prevent the tabs 128A, 128B from moving radially inward and thereby inadvertently separating the pedicle screw 102 from the reducer clip 402.

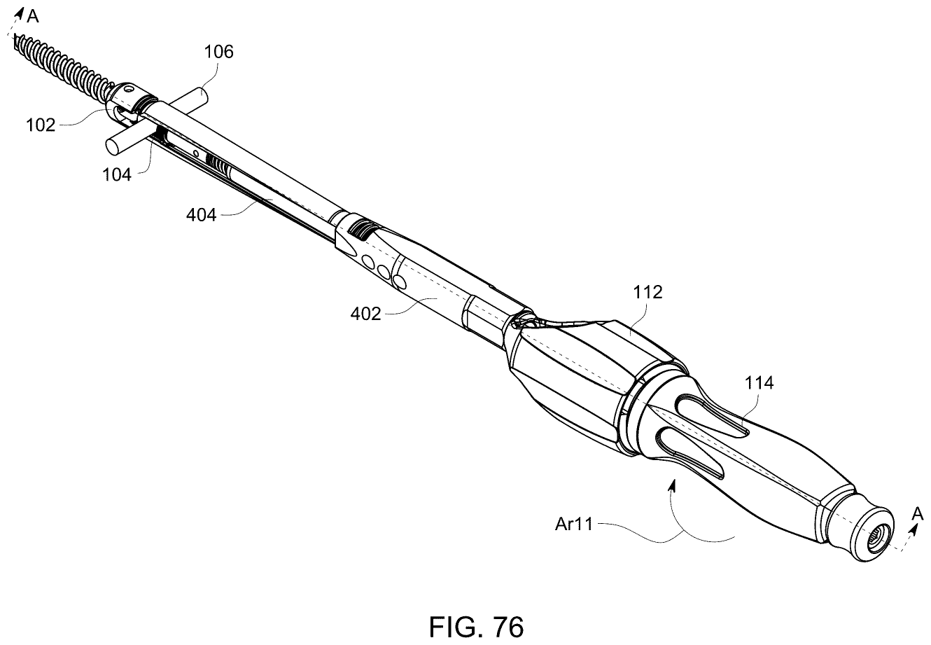

To reduce the spinal fixation rod 106 from the configuration shown in FIG. 75, the reducer handle 112 can be rotated, as shown by arrow Ar10. The interaction of the threads 180 on the reducer handle 112 with the threads 410 of the reducer clip 402 can cause distal advancement of the inserter-reducer shaft 404 and any rod 106 that it is contacting. The reducer handle 112 can be rotated until the threads bottom out and/or the shoulder 188 on the reducer handle contacts the proximal end surface 415 of the reducer clip 402, as shown in FIG. 76.

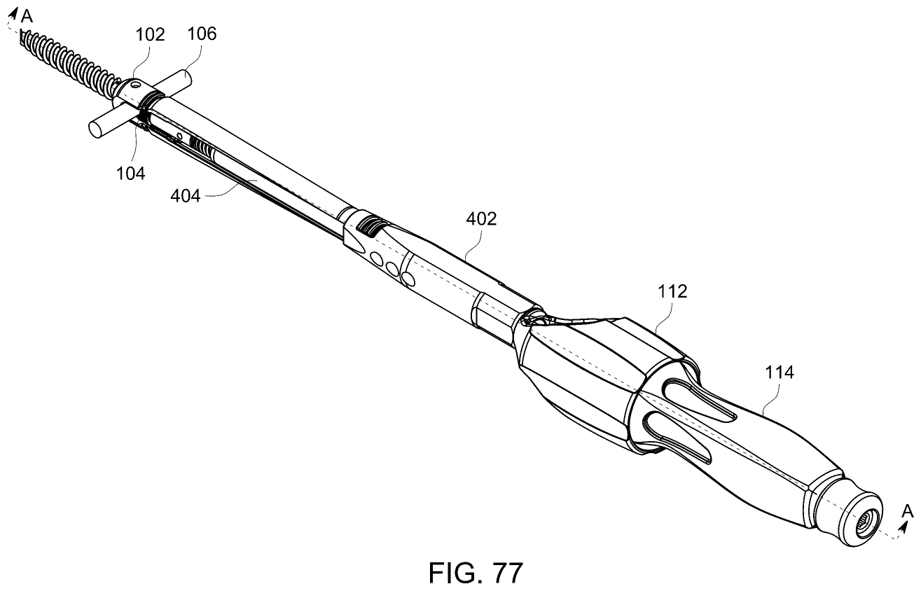

As explained above in connection with instrument 100, at the position shown in FIG. 76, the setscrew 104 can be positioned in contact with the threads 132 of the pedicle screw receiver member 118. The spring-loaded sleeve 218 can allow proximal movement of the setscrew 104 along the distal portion 216 of the inserter-reducer shaft during reduction to prevent unintentional damage by, e.g., advancing the setscrew too far distally and pressing it into the threads 132. A user can engage the setscrew 104 with the threads 132 by turning the driver handle 114, as shown by arrow Ar11 in FIG. 76. This rotation can be continued to the configuration shown in FIG. 77, wherein the setscrew 104 has completed final reduction and securement of the spinal fixation rod 106 relative to the pedicle screw 102.

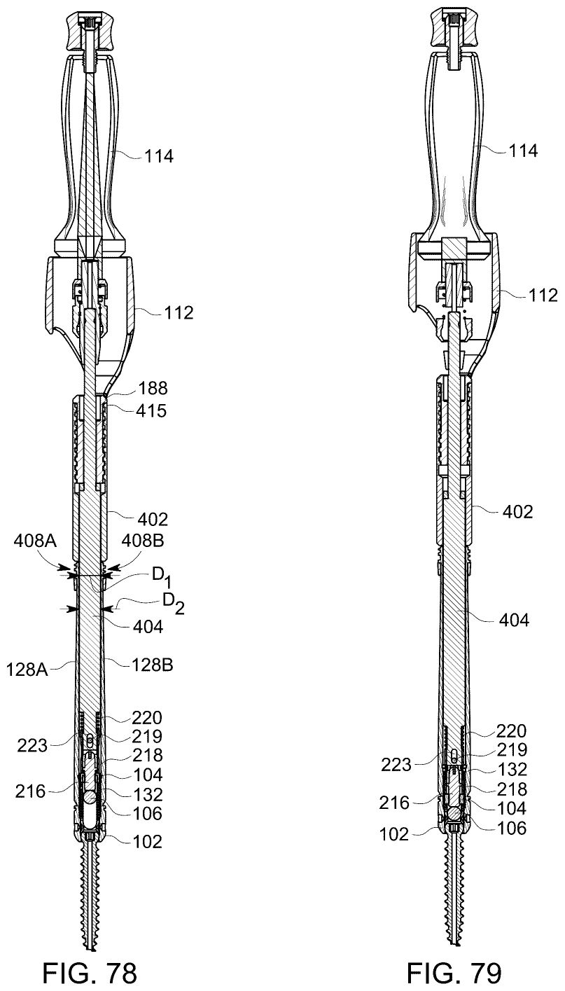

FIGS. 78 and 79 illustrate the configurations of FIGS. 76 and 77 in cross-section. In these views, the operation of the sleeve 218 and spring 220 can be seen. In particular, in the view of FIG. 76, where the reducer handle 112 has been rotated until the shoulder 188 contacts the proximal surface 415 of the reducer clip 402, shows the setscrew 104 in contact with a proximal end of the threads 132 on the pedicle screw 102. Instead of continuing to advance distally during reduction using the reducer handle 112, the setscrew 104 has translated proximally along the distal portion 216 of the inserter-reducer shaft 404 after contacting the threads 132. In doing so, it has moved the sleeve 218 proximally relative to the remainder of the shaft 404 against the bias of the spring 220. The spring 220 and sleeve 218 maintain distal pressure (of a desired magnitude) against the setscrew 104 such that it will engage with the threads 132 of the pedicle screw 102 as soon as a user begins rotating the driver handle 114.

Also shown in FIG. 78 is the diameter D₁ of the inserter-reducer shaft 404 along an intermediate portion thereof that is aligned with a distal portion of the reducer clip 402 containing the recesses 408A, 408B. As shown in the figure, the diameter D₁ substantially matches the distance or diameter D₂ between the extended tabs 128A, 128B at this position, such that the tabs cannot deflect radially inward to allow separation of the reducer clip 402 from the pedicle screw 102 as long as the shaft 404 is present.

FIG. 79 illustrates a configuration in which the driver handle 114 is being rotated to engage the threads of the setscrew 104 with the threads 132 of the pedicle screw 102. As the driver handle 114 is rotated, the setscrew 104 can move distally along the distal portion 216 of the shaft 404. As the setscrew 104 advances distally, the sleeve 218 can advance distally with it, as allowed by the pin 219 traveling within the elongate slot 223, and the spring 220 can return to a more relaxed state. In some embodiments, the distal advancement of the setscrew 104 can include overcoming the biasing force of the spring clip 229 and separating from the shaft 404 entirely. In other embodiments, the setscrew 104 can overcome the biasing force of the spring clip 229 and separate from the shaft 404 when the instrument 100 is being removed.

The reducer clip 402 described above can be more simple to manufacture when compared to the reducer clip 108, e.g., because it can be formed as a unitary or monolithic component. It contrast, however, it can require insertion and/or removal of the instrument 400 as separate components because there is no ability to separate the reducer clip 402 from the pedicle screw 102 when the inserter-reducer shaft 404 is disposed through the lumen of the reducer clip. With the reducer clip 108 and shaft 110 described above, the reduced diameter of the shaft along an intermediate portion thereof can allow for the insertion and/or removal of the instrument 100 together as a single unit.

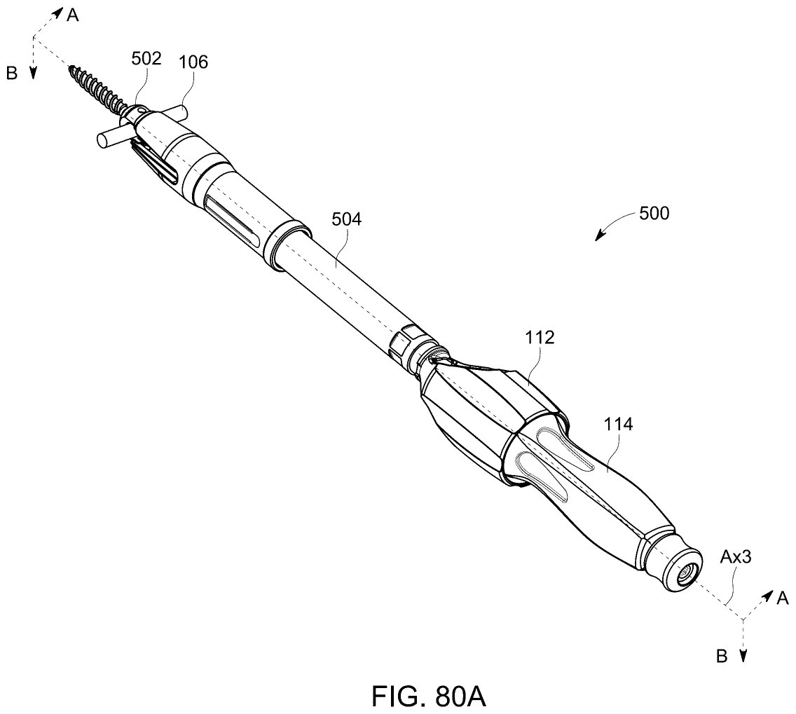

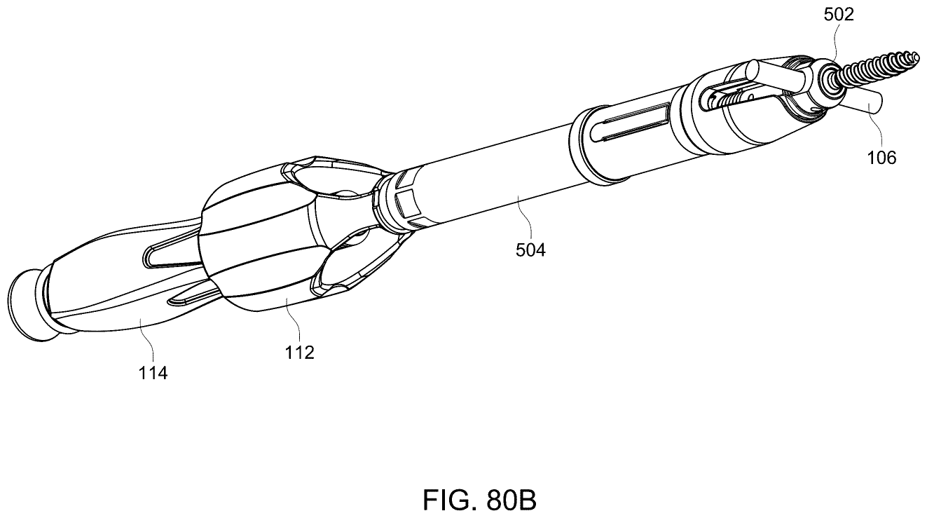

FIGS. 80A-96 illustrate another embodiment of an assembly including a pedicle screw reducer instrument 500 according to the present disclosure. The instrument 500 can be similar to the instrument 100 described above and a detailed description of every component is therefore omitted for brevity. For example, the instrument 500 can include the same inserter-reducer shaft 110, reducer handle 112, and driver handle 114 as the instrument 100 described above. In addition, the instrument 500 can be utilized in connection with the same setscrew 104 and spinal fixation rod 106 as the instrument 100 described above. The main differences relative to the instrument 100 can be its use in connection with a different pedicle screw 502 that lacks extended tabs 128A, 128B and a reducer clip in the form of a pedicle screw extension 504.

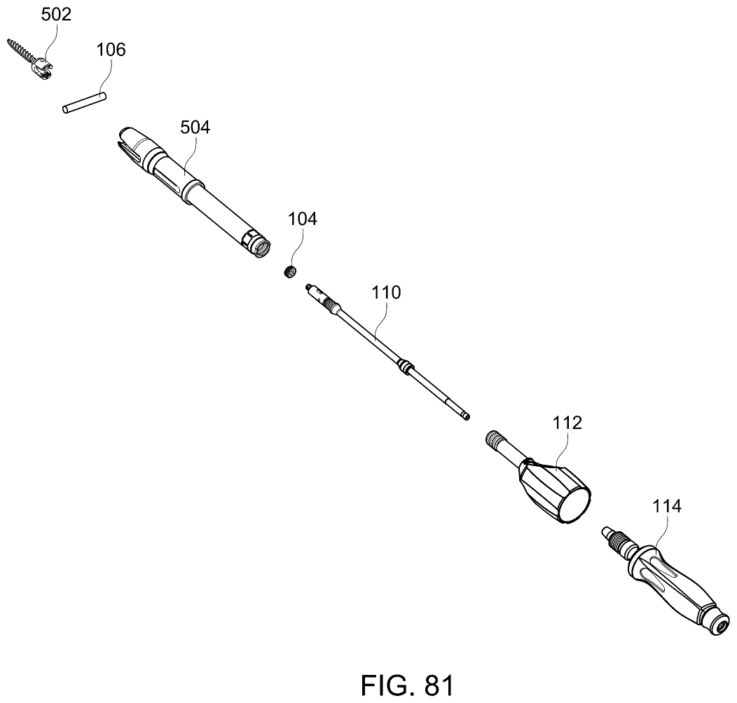

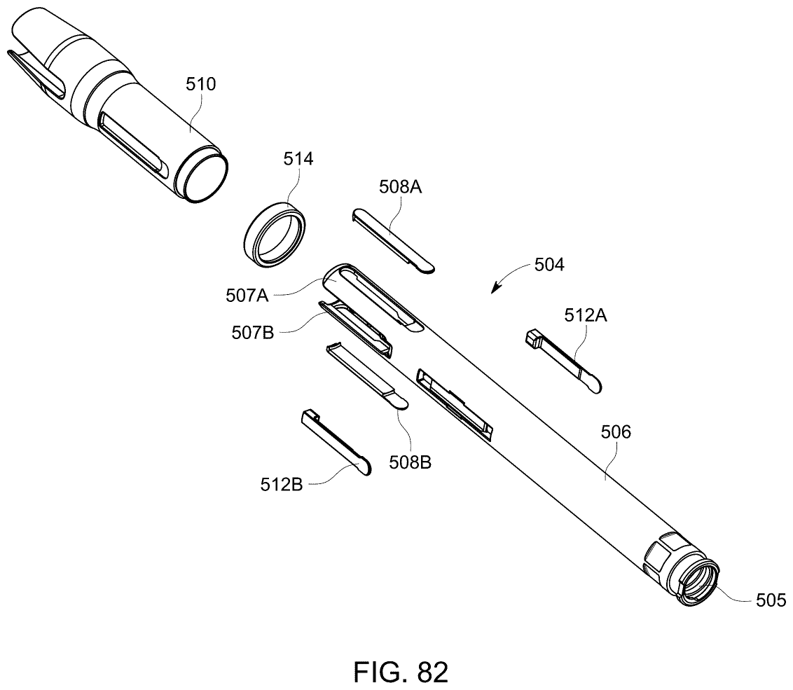

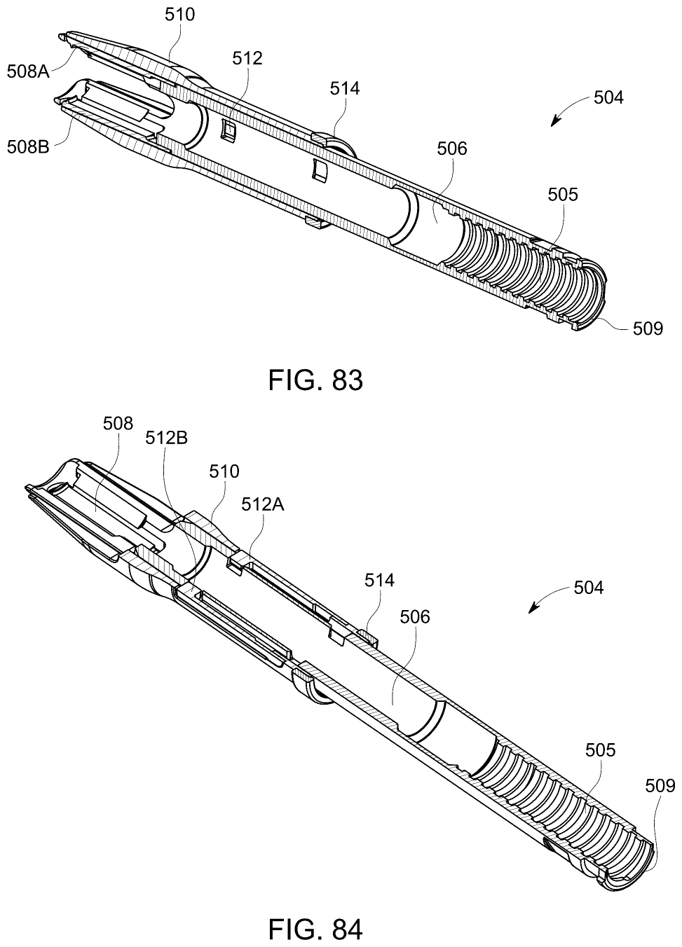

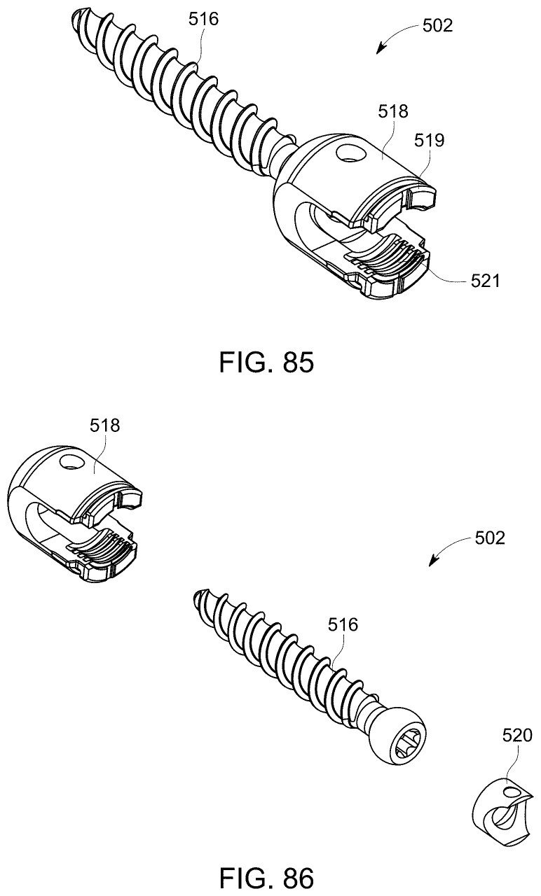

FIG. 81 illustrates an exploded view of the components in the assembly of FIGS. 80A, 80B, while FIG. 82 illustrates the components of the pedicle screw extension 504 in greater detail, while FIGS. 83 and 84 illustrate cross-sectional views of the pedicle screw extension. As shown in the figures, the pedicle screw extension 504 can include an inner sleeve 506 having a lumen formed therethrough. The inner sleeve 506 can have threads 505 formed on an inner surface along a proximal portion thereof, as well as opposed arms 507A, 507B formed at a proximal end thereof. The extension 504 can also include resilient arms 508A, 508B disposed in recessed formed in each arm 507 of the inner sleeve 506. The arms 508A, 508B can be configured to extend into the inner lumen of the sleeve 506 and engage with a feature formed on an external surface of the pedicle screw 502, such as the groove or notch 519 (see FIG. 85).

The pedicle screw extension 504 can further include an outer sleeve 510 disposed over the inner sleeve 506. The outer sleeve 510 can be configured to translate over a portion of the inner sleeve 506 and can selectively lock the extension to a pedicle screw by preventing radially outward movement of the resilient arms 508A, 508B. The outer sleeve 510 can house resilient arms 512A, 512B in recesses formed therein. These arms can extend into recesses, slots, or tracks formed in the inner sleeve 506 to define the movement of the outer sleeve 510 relative to the inner sleeve, as well as lock the outer sleeve in a proximal and/or distal position. Further, a ring 514 can be disposed over a proximal portion of the outer sleeve 510.

FIGS. 85 and 86 illustrate the pedicle screw 502 in isolation. The pedicle screw 502 can be similar to the pedicle screw 102 described above, but can lack the extended tabs 128A, 128B of the pedicle screw 102. The pedicle screw 502 can include a distally-extending implantable shank 516, a proximal receiver member 518 that polyaxially couples with a proximal head of the shank 516, and a compression member 520 disposed within the receiver member proximal to the head of the shank 516.

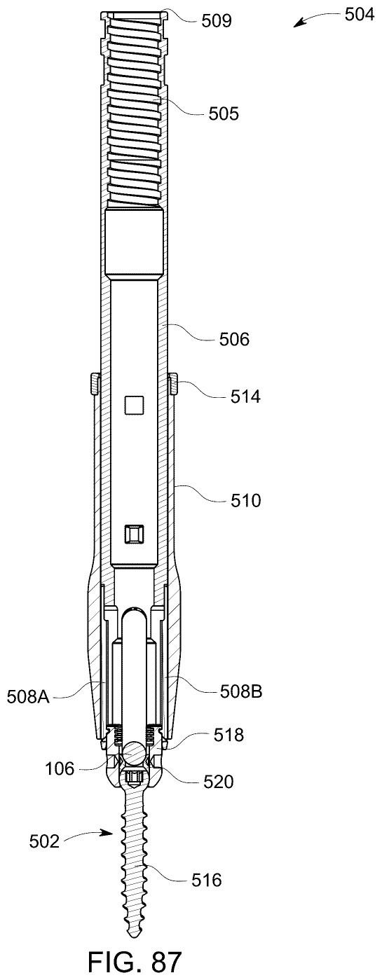

FIG. 87 illustrates the pedicle screw extension 504 coupled to the pedicle screw 502. As shown in the figure, the inner sleeve extends over a proximal portion of the pedicle screw receiver member 518. Distal portions of the resilient arms 508A, 508B extend through the recesses formed in the opposed arms 507A, 507B of the inner sleeve 506 and into the groove or notch 519 formed in an external surface of the receiver member 518. The outer sleeve 510 is shown advanced into a distal position in which it blocks any radially-outward movement of the resilient arms 508A, 508B relative to a longitudinal axis Ax3 (see FIG. 80A). In such a configuration, the arms 508A, 508B cannot move radially outward to clear the groove or notch 519, effectively locking the pedicle screw extension 504 to the pedicle screw 502.

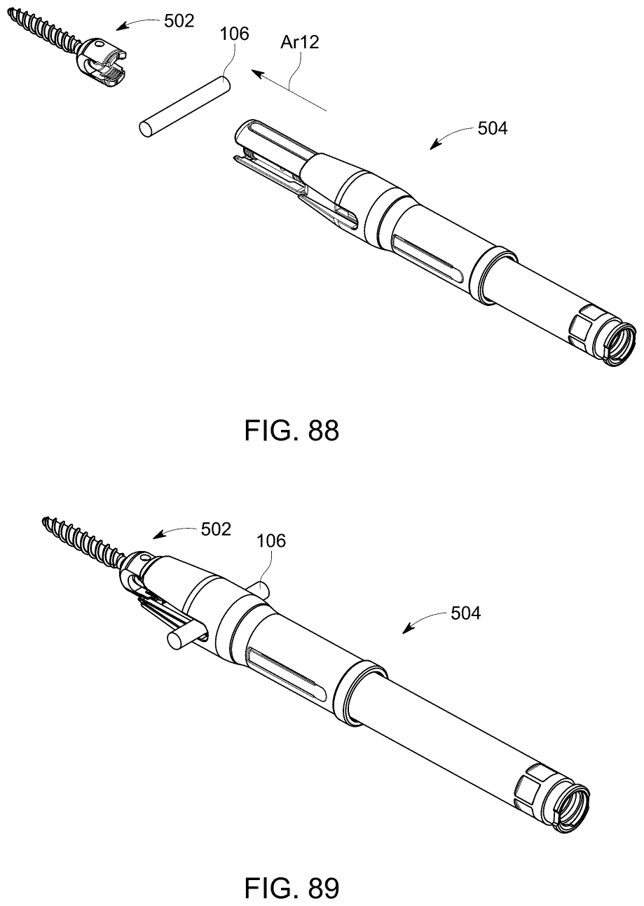

FIGS. 88-96 illustrate one method of use of the instrument 500. The method can include coupling the pedicle screw extension 504 to the pedicle screw 502 and aligning a spinal fixation rod 106 with opposed arms of the pedicle screw receiver member 518. To do this, the outer sleeve 510 can be withdrawn proximally, as shown in FIG. 88, to allow the resilient arms 508A, 508B to move radially outward as a proximal portion of the pedicle screw receiver member 518 passes between the opposed arms 507A, 507B of the inner sleeve 506. When a distal portion of the resilient arms 508A, 508B is aligned with the groove or notch 519 formed in the receiver member 518, the arms can move radially inward such that outer surfaces thereof can be flush with an outer surface of the inner sleeve 506. The outer sleeve 510 can then be advanced distally, in the direction of Ar12 in FIG. 88, to lock the pedicle screw extension 504 relative to the pedicle screw receiver member 518, as shown in FIG. 89.

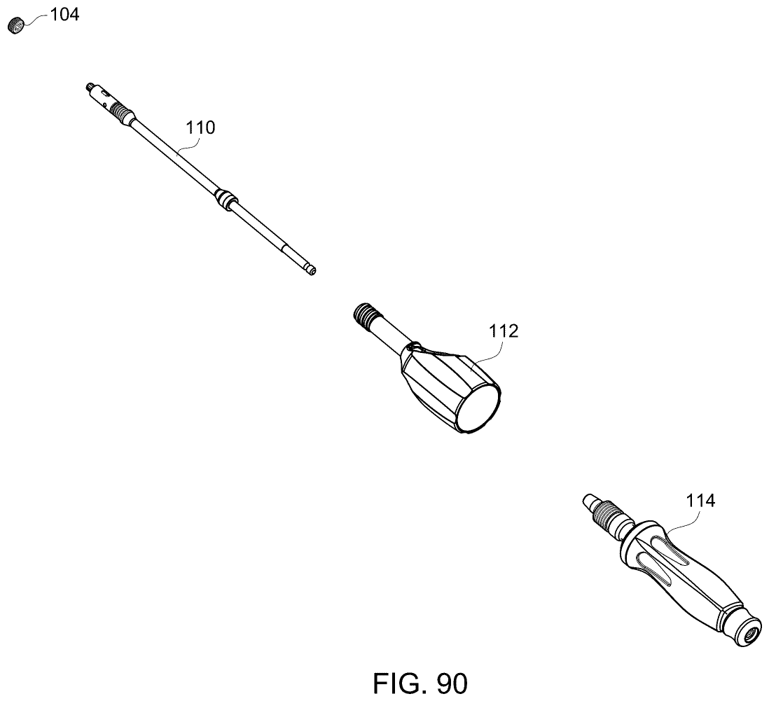

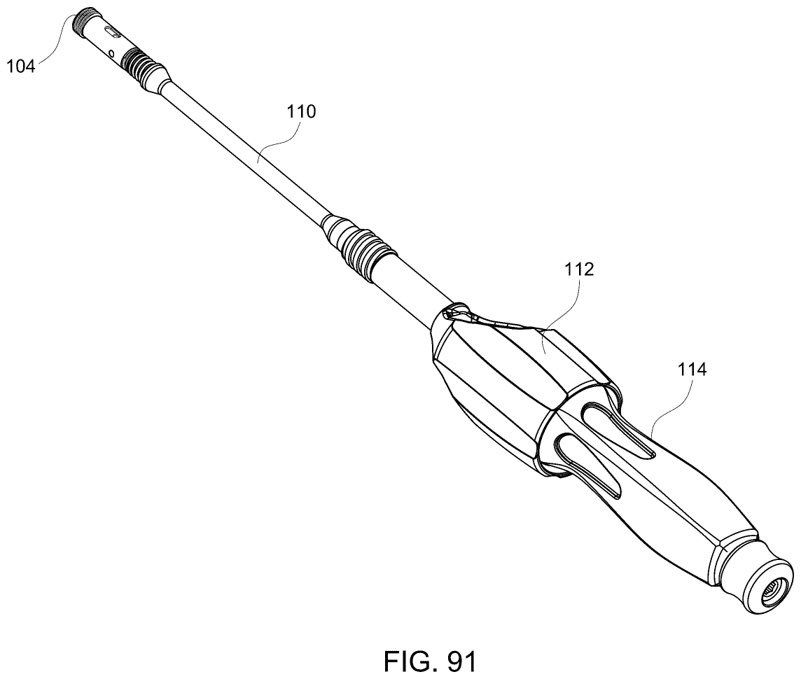

FIG. 90 illustrates the assembly of the remaining components of the instrument 500. In particular, a setscrew 104 can be loaded on a distal portion of the inserter-reducer shaft 110, and a proximal end of the shaft can be passed proximally through the lumen of the reducer handle 112. The driver handle 114 can be coupled to the proximal end of the shaft 110 that extends proximally through the reducer handle 112 to form the assembly shown in FIG. 91.

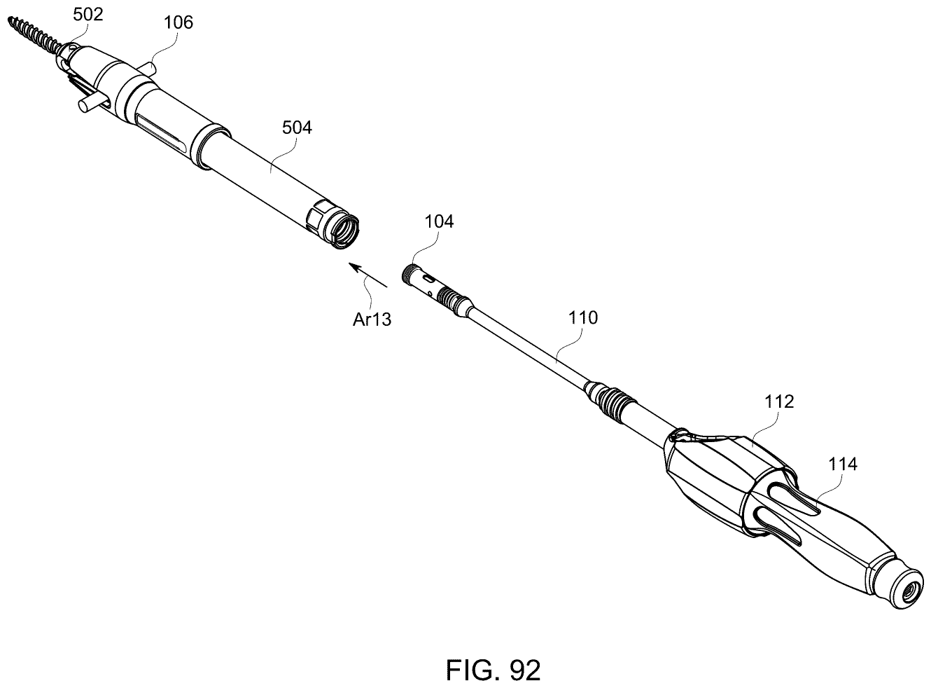

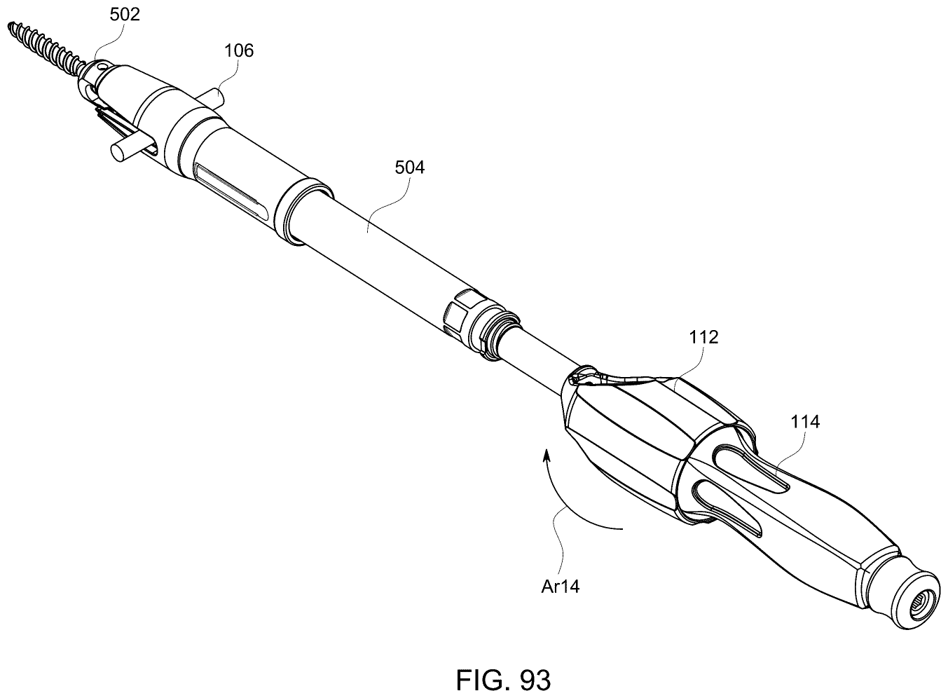

FIG. 92 illustrates a step wherein the assembly of FIG. 91 is disposed near a proximal end of the pedicle screw extension 504. The setscrew 104 and associated components can be advanced distally relative to the pedicle screw extension 504, as shown by arrow Ar13 in FIG. 92, to the configuration of FIG. 93 wherein the threads of the reducer handle 112 contact the threads 505 of the pedicle screw extension 504.

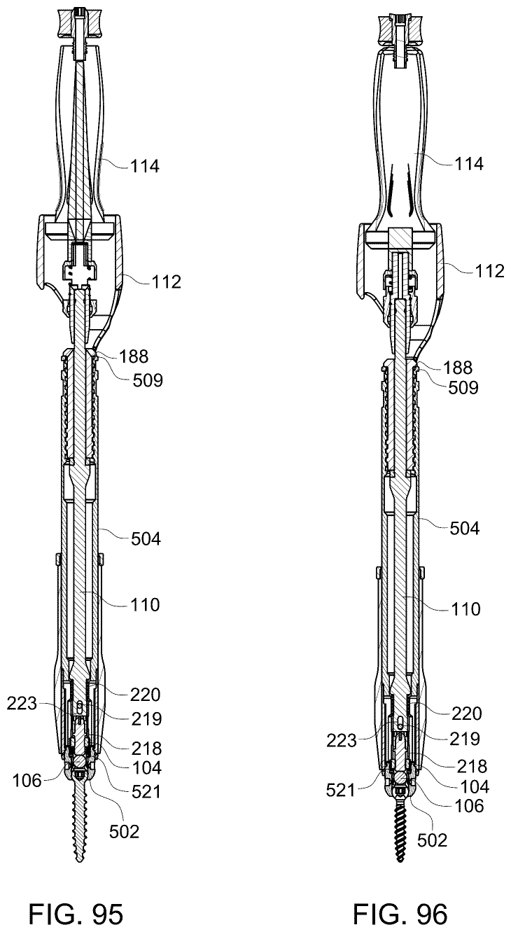

The reducer handle 112 can be rotated, as shown by arrow Ar14 in FIG. 93, to distally advance the reducer handle 112, shaft 110, and setscrew 104 relative to the pedicle screw extension 504. This can also distally reduce the rod 106 in contact with a distal end of the shaft 110. As noted above, the reducer handle 112 can be rotated until the distal-facing shoulder 188 contacts a proximal surface 509 of the pedicle screw extension 504 or, in some embodiments, until the rod 106 is fully reduced into the receiver member 518 (which may occur before the distal-facing shoulder 188 contacts the proximal surface 509). At such a point, as shown in FIG. 95, the setscrew 104 can be in contact with the threads 521 of the pedicle screw receiver member 518. As noted above, the spring-loaded sleeve 218 of the shaft 110 can exert a distally-directed force on the setscrew 104 to maintain it in contact with the threads 521 of the receiver member 518 while allowing the setscrew to move proximally along a distal portion of the shaft to prevent damage by over-advancing the setscrew during reduction via the reducer handle 112.

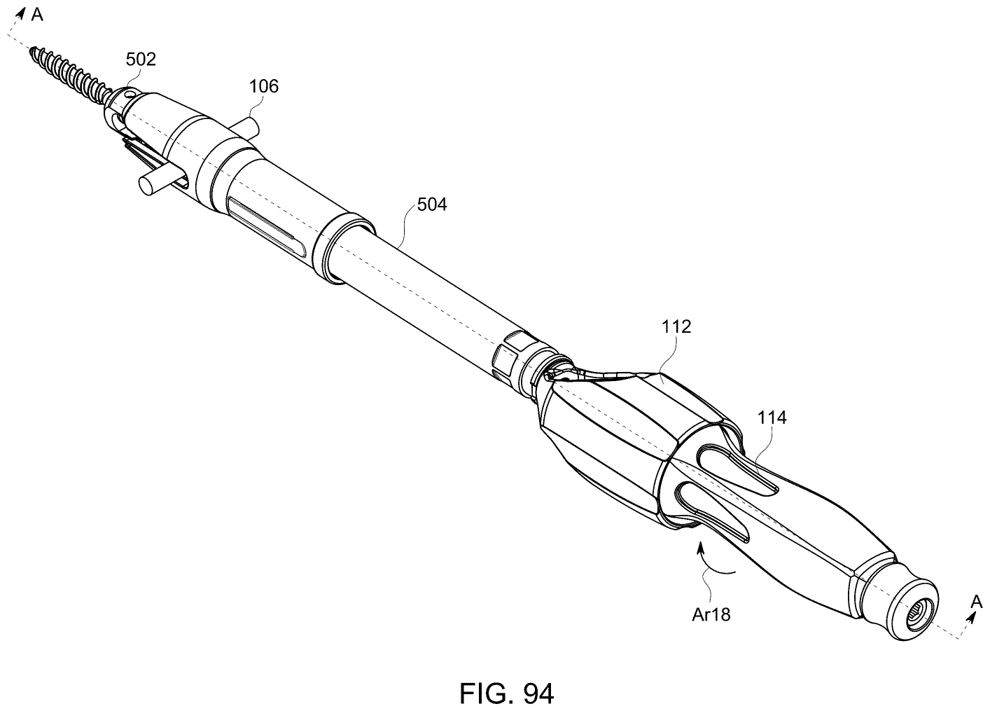

The setscrew 104 can be engaged with the threads 521 of the pedicle screw receiver member 518 by rotating the driver handle 114, as shown by arrow Ar18 in FIG. 94. This can advance the setscrew 104 distally along the shaft 110, as shown in FIG. 96, to perform final reduction and securing of the spinal fixation rod 106 relative to the pedicle screw 502.

Note that the above-described inserter-reducer shaft, which utilizes the spring-loaded sleeve to distally bias a setscrew disposed along a distal portion thereof while allowing proximal translation against the biasing force, can be utilized in a variety of embodiments with pedicle screws that include or lack extended tabs. Such a configuration of setscrew inserter can allow for some play when introducing the setscrew and facilitate easier starting of a threaded engagement between the setscrew and the pedicle screw. In addition, utilizing a single shaft for both reduction and setscrew deployment can minimize the amount and bulk of instrumentation, thereby improving various aspects of a procedure, such as the ability for a user to visualize a surgical site, a position of the setscrew and operation of the instrument, etc. For example, separate rod reduction instruments can contact a rod at positions outboard from the setscrew, thereby blocking the ability for a user to directly visualize the setscrew during delivery.

Further, the above-described configurations can be utilized in a variety of reducer instruments, including instruments where a reducer handle may or may not bottom out against a reducer clip to achieve maximum reduction of a spinal fixation rod. As noted above, in certain embodiments the distal portion of an inserter-reducer shaft can extend distally beyond a setscrew disposed thereon and contact a rod directly. In some embodiments, distal advancement of the shaft can achieve full or maximum reduction of the rod, pressing it fully into a rod-receiving seat of a pedicle screw. The reducer instruments can be configured such that, having achieved full reduction, the setscrew is in contact with the threads of the pedicle screw (with the spring-loaded sleeve urging it distally) and can be threaded into engagement with the pedicle screw by rotating the inserter-reducer shaft.

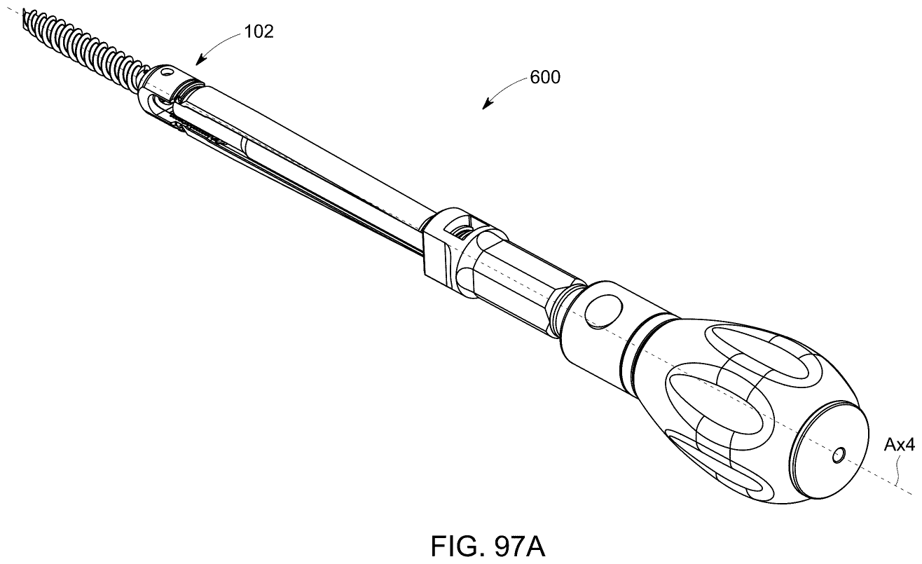

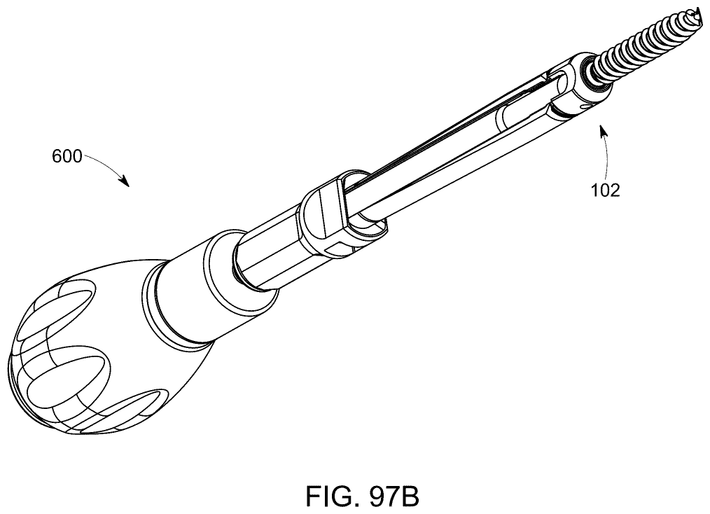

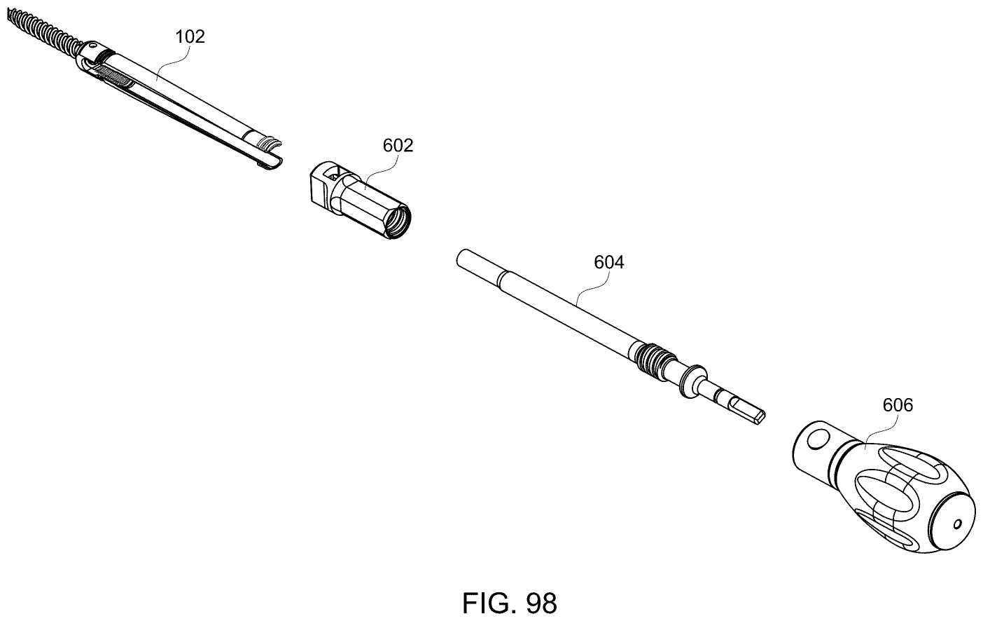

FIGS. 97A-110 illustrate another embodiment of an assembly including a pedicle screw reducer instrument 600 according to the present disclosure. The instrument 600 can be similar to the other instruments described above and a detailed description of every component is therefore omitted for brevity. For example, the instrument 600 can be utilized in connection with the same pedicle screw 102 and spinal fixation rod 106 as the instrument 100 described above. The instrument 600, however, can be configured for use in reducing a spinal fixation rod 106 at an “adjacent level” to facilitate the introduction of a setscrew 104 using another instrument disposed nearby. For example, in a spinal fixation construct utilizing pedicle screws implanted in a plurality of vertebra or vertebral levels, the instrument 600 can be utilized to reduce or distally advance a rod into a pedicle screw at a first vertebra or level, which can facilitate the introduction of a setscrew into a pedicle screw at an adjacent, second vertebra or level to capture, reduce, and/or secure the rod. FIG. 98 illustrated the components of the instrument 600 in an exploded view. The instrument 600 can include a reducer clip 602, a reducer shaft 604, and a reducer handle 606.

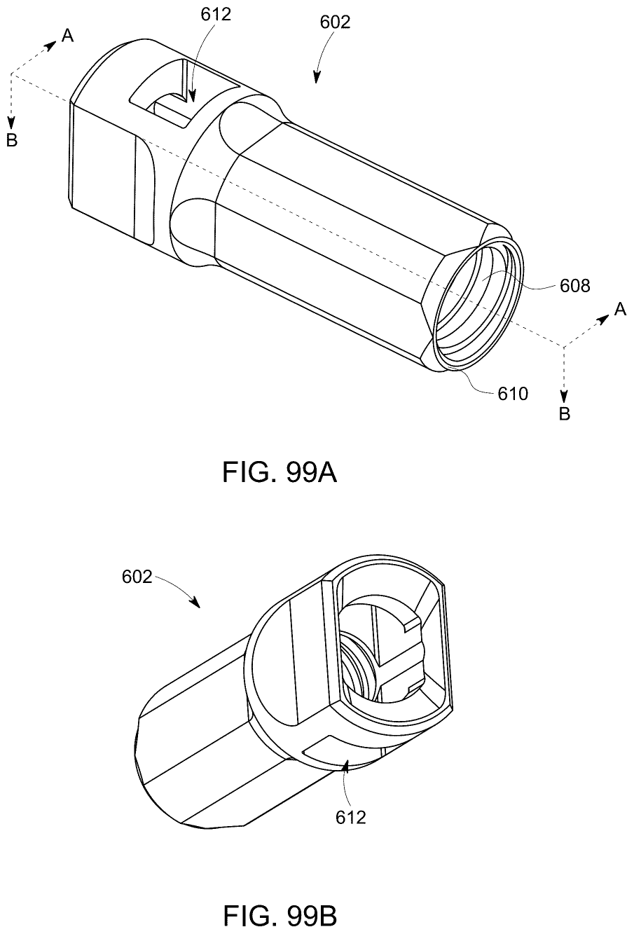

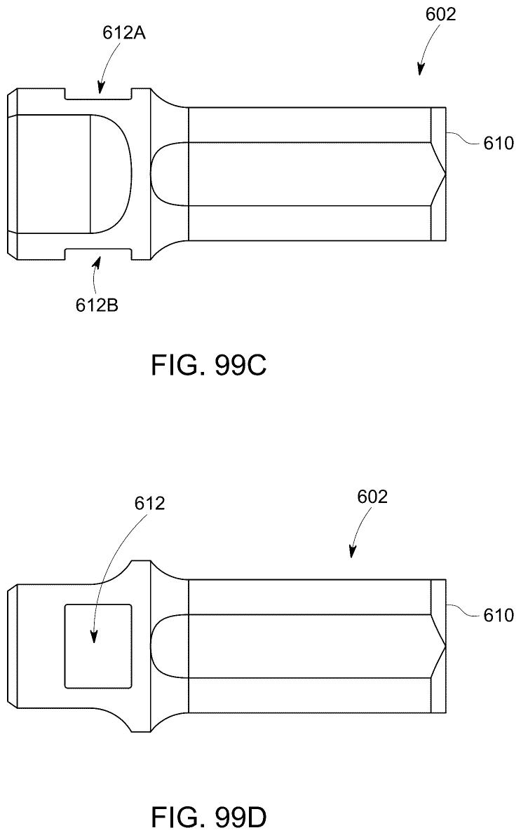

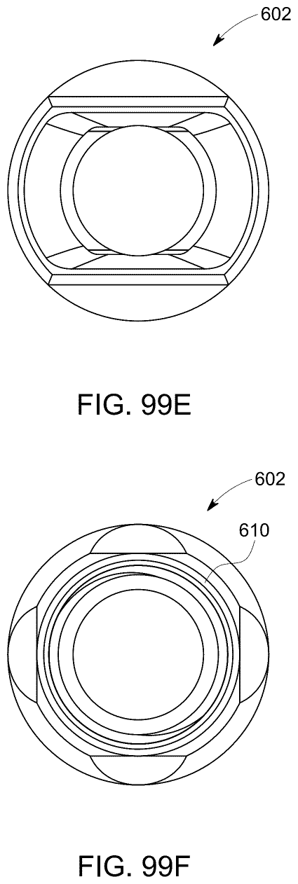

FIGS. 99A-101 illustrate the reducer clip 602 in isolation. The reducer clip 602 can be similar to the reducer clip 402 described above. For example, the reducer clip 602 can be a unitary or monolithic component with a lumen extending therethrough having threads 608 that extend from a proximal end 610 thereof and recesses 612A, 612B formed in a sidewall along a distal portion thereof. The recesses 612A, 612B can be configured to receive the threads 134 and/or flange 135 formed on an external surface of a proximal portion of the extended tabs 128A, 128B of the pedicle screw 102.

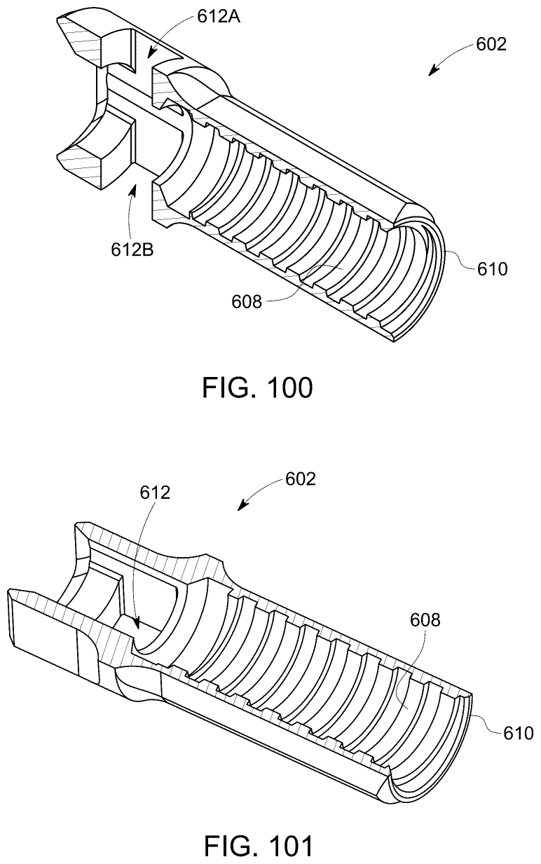

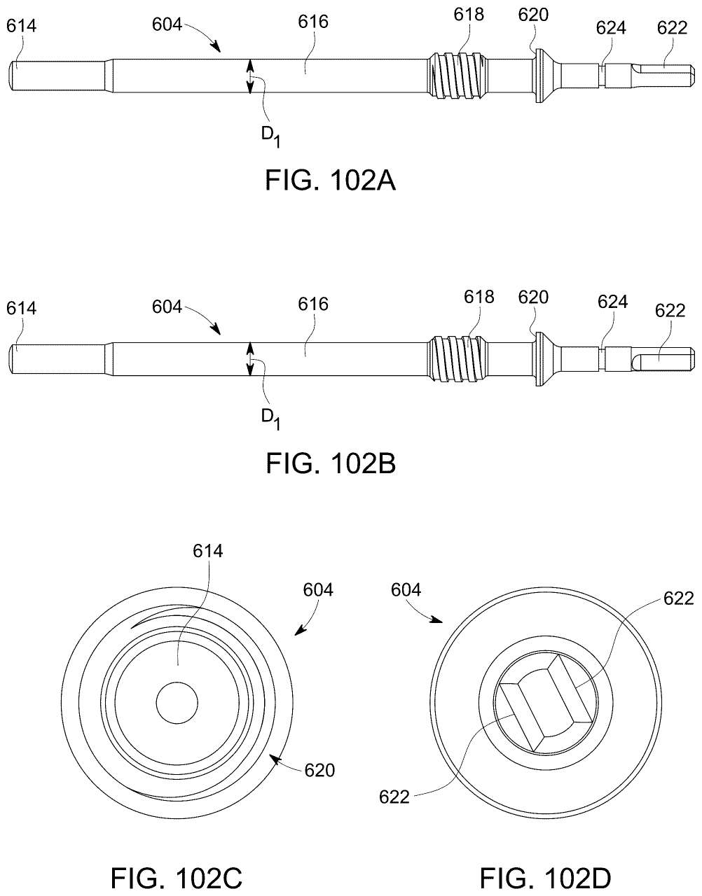

FIGS. 102A-102D illustrate the reducer shaft 604 in isolation. Given that the reducer shaft 604 does not introduce or deploy a set screw and instead only pushes on a rod 106 to provisionally reduce it, the shaft can have a simpler, blunt distal end 614 without the spring-loaded sleeve 218 and other components found on the inserter-reducer shafts described above. The shaft 604 can also include an intermediate portion 616 with an outer diameter D₁ that is substantially the same as an inner diameter or distance between the opposed extended tabs 128A, 128B of the pedicle screw 102. In this manner, the presence of the shaft 604 disposed between the extended tabs 128A, 128B can prevent the radially-inward movement of the tabs that might allow separation of the reducer clip 604 from the pedicle screw 102, similar to the inserter-reducer shaft 404 and reducer clip 402 described above.

The reducer shaft 604 can also include threads 618 formed along a portion thereof that can be configured to interface with the threads 608 of the reducer clip 602 to perform rod reduction. A distal-facing shoulder 620 can be formed proximal to the threads 618 and can be configured to contact a proximal end 610 of the reducer clip 602 to stop any further reduction. A proximal portion of the shaft 604 can include any combination of surfaces configured to facilitate coupling to the reducer handle 606, such as one or more flats 622 and/or one or more at least partial circumferential grooves 624.

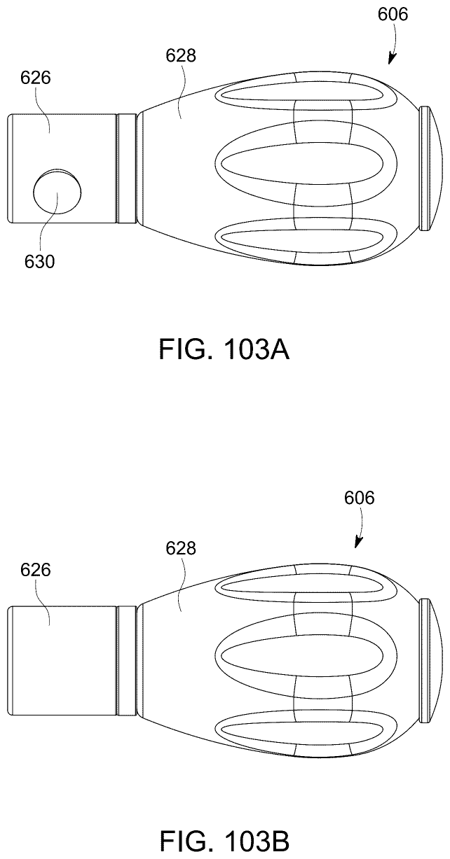

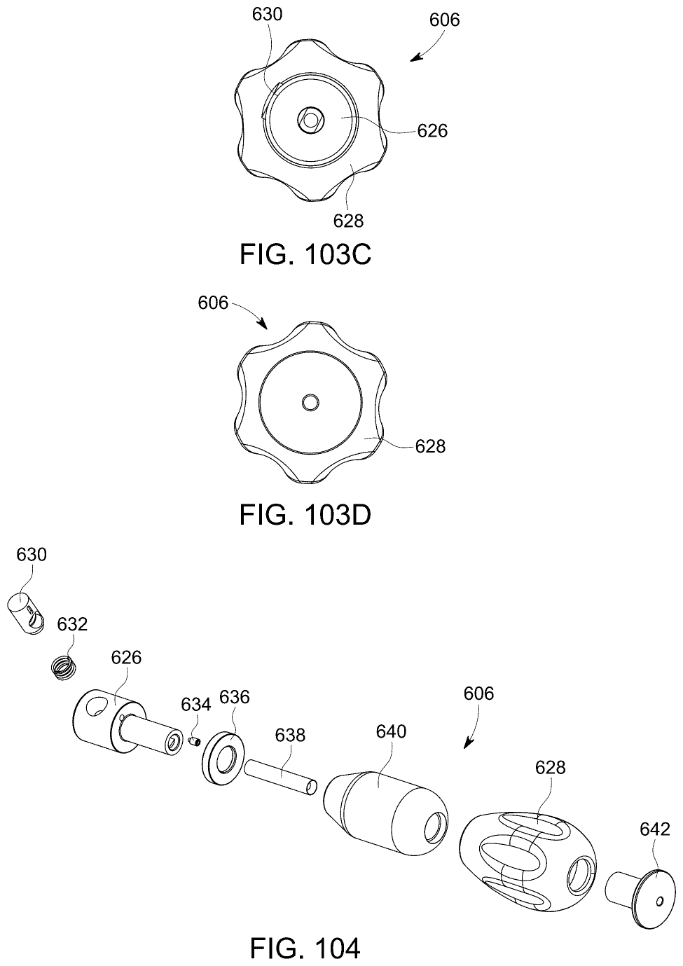

FIGS. 103A-104 illustrate the reducer handle 606 in isolation. The reducer handle 606 can include a distal body 626 with a distal-facing recess configured to receive a proximal portion of the reducer shaft 604, as well as a proximal handle 628 coupled thereto. The reducer handle 606 can also include a spring-loaded button 630 that can be utilized to selectively lock the reducer handle against separation from the reducer shaft 604.

The exploded view of FIG. 104 illustrates additional components of the reducer handle 606, including a spring 632 to bias the button 630, as well as a pin 634 that can be disposed within a bore formed in the body 626 and can extend into an elongate slot formed in the button 630 to limit its travel and prevent ejection from the body 626 by the force of the spring 632. A washer 636 can retain the pin 634 within the body 626. Also shown is a shaft 638, core 640, and cap 642 that can form components of the proximal handle 628.

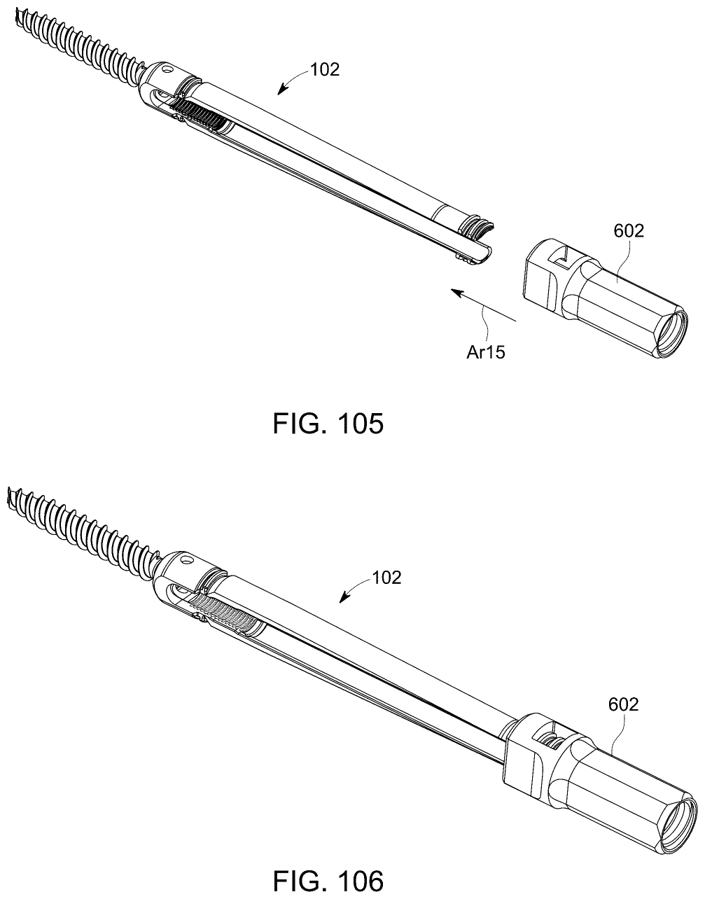

FIGS. 105-110 illustrate a method of operation of the reducer instrument 600. In FIG. 105, the reducer clip 602 can be positioned near a proximal end of the pedicle screw 102. The reducer clip 602 can be advanced distally, in the direction of arrow Ar15, to pass a distal portion of the reducer clip 602 over a proximal portion of the pedicle screw 102. To do this, proximal ends of the extended tabs 128A, 128B of the pedicle screw 102 can be squeezed together or moved radially inward relative to a longitudinal axis Ax4 (see FIG. 97A) to allow them to pass into the lumen of the reducer clip 602. When the threads 134 and/or flange 135 formed on the external surface of the extended tabs 128A, 128B are aligned with the recesses 612A, 612B of the reducer clip 602, the extended tabs 128A, 128B can move radially outward and return to their resting position, thereby coupling the reducer clip to the pedicle screw, as shown in FIG. 106.

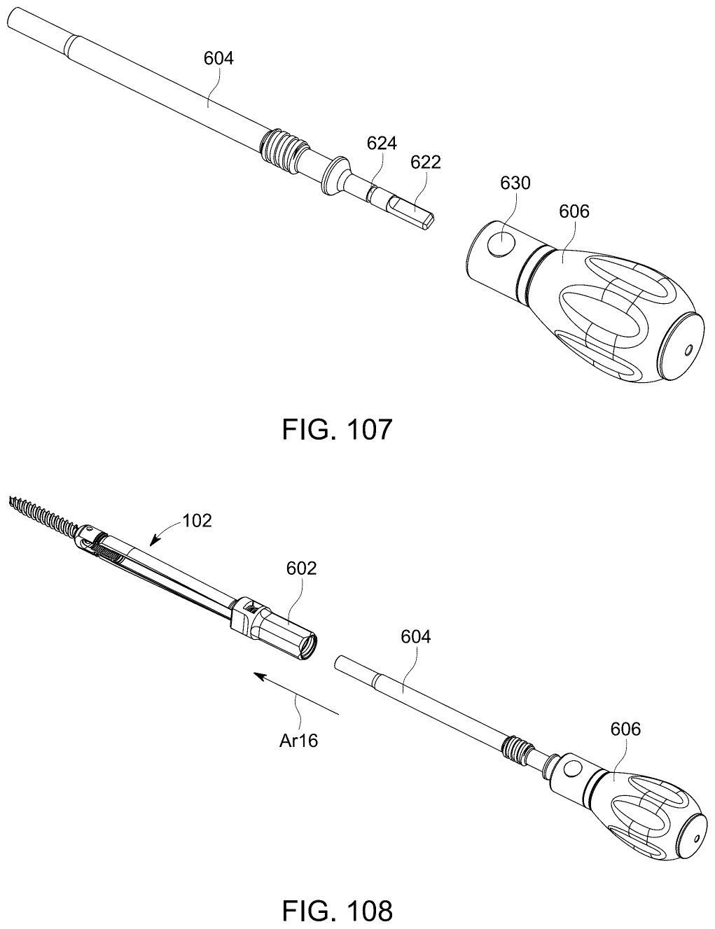

As shown in FIG. 107, the reducer shaft 604 can be coupled to the reducer handle 606 by inserting a proximal end of the shaft into the distal-facing recess of the handle. The spring-loaded button 630 can seat itself in the at least partial circumferential groove 624 to selectively lock the two components together, and a flat surface of the recess in the handle can interface with the flat surface 622 of the shaft to prevent relative rotation between the shaft and handle.

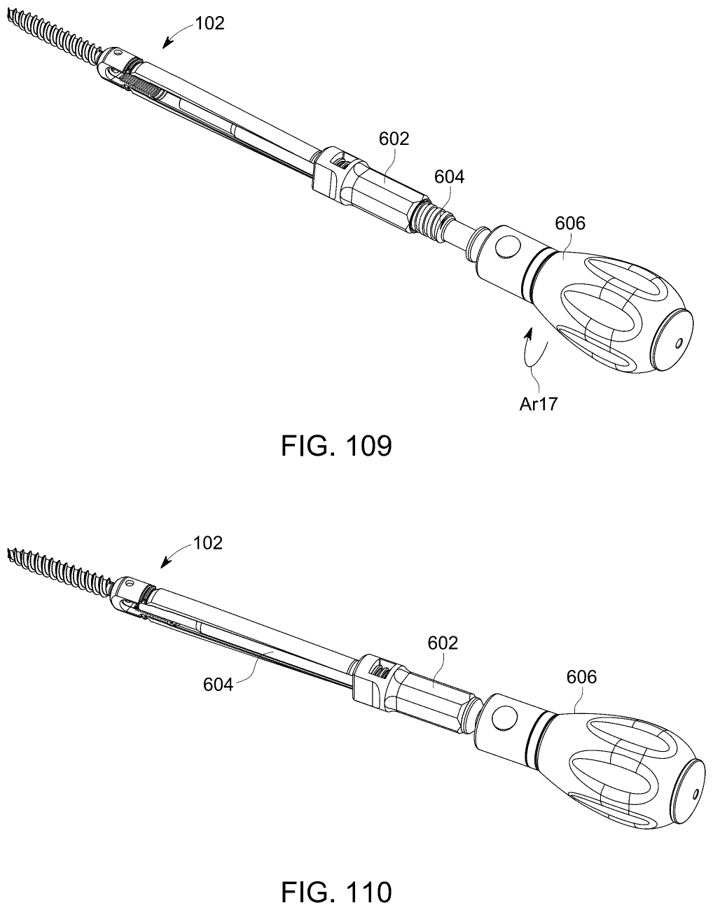

As shown in FIG. 108, the shaft 604 can be positioned near a proximal end of the reducer clip 602 and advanced distally relative thereto, as shown by arrow Ar16. The distal end of the shaft 604 can be inserted through the lumen of the reducer clip 602 until threads 618 of the shaft contact the threads 608 of the reducer clip, as shown by FIG. 109. From here, the reducer handle 606 can be rotated, as shown by arrow Ar17, to engage the threads 618 of the shaft 604 with the threads 608 of the reducer clip 602 and thereby distally advance the shaft, along with any rod in contact with its distal end, distally relative to the reducer clip and pedicle screw 102. The reducer handle 606 can be rotated until the distal-facing shoulder 620 contacts the proximal end 610 of the reducer clip 602.

As noted above, the “adjacent level” reducer instrument 600 can be utilized in connection with a pedicle screw implanted at a first vertebra or vertebral level in order to provisionally reduce a rod relative to the pedicle screw. This can have the effect of also reducing the rod relative to a pedicle screw implanted at an adjacent, second vertebra or vertebral level. Reducing the rod in this manner can facilitate a user introducing a setscrew into the pedicle screw implanted at the second vertebra or vertebral level in order to further reduce and securely capture the rod. Once the setscrew is utilized to maintain a position of the rod relative to the second pedicle screw, the instrument 600 can be removed from the first pedicle screw and a setscrew can be inserted into the first pedicle screw. Removing the instrument 600 can require the separate removal of the reducer shaft 604 and reducer clip 602. This can be because the presence of the reducer shaft 604, with its enlarged diameter D₁ along an intermediate portion thereof, between the extended tabs 128A, 128B of the pedicle screw can prevent radially-inward movement of the extended tabs that can be necessary to separate the pedicle screw from the reducer clip 602.

Various devices and methods disclosed herein can be used in minimally invasive surgery and/or open surgery. While various devices and methods disclosed herein are generally described in the context of surgery on a human patient, the methods and devices disclosed herein can be used in any of a variety of surgical procedures with any human or animal subject, or in non-surgical procedures.

Various devices disclosed herein can be constructed from any of a variety of known materials. Example materials include those that are suitable for use in surgical applications, including metals such as stainless steel, titanium, titanium nitride, nickel, cobalt, chrome, cobalt-chromium, or alloys and combinations thereof, polymers such as PEEK, ceramics, carbon fiber, and so forth. The various components of the devices disclosed herein can be rigid or flexible. In addition, one or more of the components or devices disclosed herein can be formed as monolithic or unitary structures, e.g., formed from a single continuous material, or can be formed from separate components coupled together in a variety of manners that either facilitate or discourage subsequent separation. One or more components or portions of the device can be formed from a radiopaque material to facilitate visualization under fluoroscopy and other imaging techniques, or from a radiolucent material so as not to interfere with visualization of other structures. Example radiolucent materials include carbon fiber and high-strength polymers. Further, various methods of manufacturing can be utilized, including 3D printing or other additive manufacturing techniques, as well as more conventional manufacturing techniques, including molding, stamping, casting, machining, etc.

Various devices or components disclosed herein can be designed to be disposed of after a single use, or they can be designed to be used multiple times. In either case, however, various devices or components can be reconditioned for reuse after at least one use. Reconditioning can include any combination of the steps of disassembly, followed by cleaning or replacement of particular pieces, and subsequent reassembly. In particular, a device or component can be disassembled, and any number of the particular pieces or parts thereof can be selectively replaced or removed in any combination. Upon cleaning and/or replacement of particular parts, the device or component can be reassembled for subsequent use either at a reconditioning facility, or by a surgical team immediately prior to a surgical procedure. Reconditioning of a device or component can utilize a variety of techniques for disassembly, cleaning/replacement, and reassembly. Use of such techniques, and the resulting reconditioned device or component, are within the scope of the present disclosure.

Various devices or components described herein can be processed before use in a surgical procedure. For example, a new or used device or component can be obtained and, if necessary, cleaned. The device or component can be sterilized. In one sterilization technique, the device or component can be placed in a closed and sealed container, such as a plastic or TYVEK bag. The container and its contents can be placed in a field of radiation that can penetrate the container, such as gamma radiation, x-rays, or high-energy electrons. The radiation can kill bacteria on the device or component and in the container. The sterilized device or component can be stored in the sterile container. The sealed container can keep the device or component sterile until it is opened in the medical facility. Other forms of sterilization are also possible, including beta or other forms of radiation, ethylene oxide, steam, or a liquid bath (e.g., cold soak). Certain forms of sterilization may be better suited to use with different devices or components, or portions thereof, due to the materials utilized, the presence of electrical components, etc.

In this disclosure, articles “a” and “an” are used to refer to one or to more than one (i.e., at least one) of the grammatical object of the article. By way of example, “an element” means at least one element and can include more than one element. The term “about” is used to provide flexibility to a numerical range endpoint by providing that a given value may be “slightly above” or “slightly below” the endpoint without affecting the desired result. The use herein of the terms “including,” “comprising,” or “having,” and variations thereof, is meant to encompass the elements listed thereafter and equivalents thereof, as well as additional elements. As used herein, “and/or” refers to and encompasses any and all possible combinations of one or more of the associated listed items, as well as the lack of combinations where interpreted in the alternative (“or”). Further, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B,” “one or more of A and B,” and “A and/or B” are each intended to mean “A alone, B alone, or A and B together.” A similar interpretation is also intended for lists including three or more items. For example, the phrases “at least one of A, B, and C,” “one or more of A, B, and C,” and “A, B, and/or C” are each intended to mean “A alone, B alone, C alone, A and B together, A and C together, B and C together, or A and B and C together.” In addition, use of the term “based on,” is intended to mean, “based at least in part on,” such that an un-recited feature or element is also permissible.

To the extent that linear, circular, or other dimensions are used in the description of the disclosed devices and methods, such dimensions are not intended to limit the types of shapes that can be used in conjunction with such devices and methods. Equivalents to such dimensions can be determined for different geometric shapes, etc. Further, like-numbered components of the embodiments can generally have similar features. Still further, sizes and shapes of the devices, and the components thereof, can depend at least on the anatomy of the subject in which the devices will be used, the size and shape of objects with which the devices will be used, and the methods and procedures in which the devices will be used.

The figures provided herein are not necessarily to scale. Still further, to the extent arrows are used to describe a direction of movement, these arrows are illustrative and in no way limit the direction that the respective component can or should be moved. Other movements and directions may be possible to create the desired result in view of the present disclosure.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

Further features and advantages based on the above-described embodiments are possible and within the scope of the present disclosure. Accordingly, the disclosure is not to be limited by what has been particularly shown and described. All publications and references cited herein are expressly incorporated herein by reference in their entirety, except for any definitions, subject matter disclaimers, or disavowals, and except to the extent that the incorporated material is inconsistent with the express disclosure herein, in which case the language in this disclosure controls.

Examples of the above-described embodiments can include the following:
1. A surgical system, comprising:
   a reducer clip having a lumen extending from a proximal end to a distal end thereof, a recess formed in a sidewall of the reducer clip along a distal portion thereof, and threads formed in the sidewall of the reducer clip along a proximal portion thereof;
   a shaft disposed through the lumen of the reducer clip;
   a reducer handle having a lumen extending from a proximal end to a distal end thereof and threads formed along an outer distal portion thereof, the reducer handle threads interfacing with the threads formed along the proximal portion of the reducer clip; and
   a driver handle coupled to a proximal portion of the shaft extending through the reducer handle.
2. The system of example 1, wherein the driver handle extends proximal to the reducer handle.
3. The system of any of examples 1 to 2, wherein the reducer clip is a monolithic component.
4. The system of any of examples 1 to 2, wherein the reducer clip further comprises a locking sleeve with a locking tab at a distal end thereof that is configured to translate relative to another portion of the reducer clip.
5. The system of example 4, wherein the reducer clip further comprises a biasing element that urges the locking sleeve distally relative to another portion of the reducer clip.
6. The system of any of examples 1 to 5, wherein the reducer clip further comprises opposed tabs extending radially away from a longitudinal axis of the reducer clip.
7. The system of any of examples 1 to 6, wherein the reducer clip further comprises a flat formed on an outer proximal portion thereof.
8. The system of any of examples 1 to 7, wherein the shaft further comprises a distal portion configured to rotationally drive a setscrew, a sleeve disposed around the shaft over at least part of the distal portion thereof, and a biasing element disposed proximal to the sleeve and configured to urge the sleeve distally relative to the shaft.
9. The system of any of examples 1 to 8, wherein the shaft further comprises a proximal-facing shoulder.
10. The system of any of examples 1 to 9, wherein the shaft further comprises a flat formed along a proximal portion thereof.
11. The system of any of examples 1 to 10, wherein the reducer handle further comprises a distal-facing shoulder disposed proximal to the threads that is configured to contact a proximal-facing portion of the reducer clip and prevent further distal advancement of the reducer handle relative to the reducer clip.
12. The system of any of examples 1 to 11, wherein the reducer handle further comprises a cylindrical proximal portion defining a proximal-facing recess.
13. The system of any of examples 1 to 11, wherein the reducer handle further comprises opposed paddles extending radially away from a longitudinal axis of the reducer handle and defining a gap therebetween at a proximal end of the reducer handle.
14. The system of any of examples 1 to 13, wherein the reducer clip comprises a bone anchor extension having an inner sleeve with opposed distally-extending arms and a threaded proximal portion, as well as an outer sleeve disposed over the inner sleeve and configured to translate relative to the inner sleeve to selectively lock the bone anchor extension to a bone anchor.
15. The system of any of examples 1 to 14, further comprising a bone anchor, wherein the reducer clip is configured to couple to a proximal portion of the bone anchor.
16. A reducer clip, comprising:
   a distal sleeve having a lumen extending from a proximal end to a distal end thereof and a recess formed in a sidewall of the distal sleeve along a distal portion thereof;
   a proximal sleeve having a lumen extending from a proximal end to a distal end thereof and threads formed in a sidewall of the proximal sleeve along a proximal portion thereof; and
   a locking sleeve having a lumen extending from a proximal end to a distal end thereof and a locking tab at the distal end thereof;
   wherein a distal portion of the proximal sleeve is received within a proximal portion of the distal sleeve;
   wherein a distal portion of the locking sleeve is received within the proximal portion of the distal sleeve;
   wherein the locking sleeve is axially disposed between the distal sleeve and the proximal sleeve and configured to axially translate relative thereto.
17. The reducer clip of example 16, further comprising a biasing element disposed between the locking sleeve and the proximal sleeve that is configured to distally bias the locking sleeve relative to the proximal sleeve.
18. The reducer clip of any of examples 16 to 17, wherein the distal sleeve includes opposed recesses formed in the sidewall of the distal sleeve along a distal portion thereof and the locking sleeve includes opposed locking tabs at the distal end thereof.
19. The reducer clip of any of examples 16 to 18, wherein the locking sleeve further comprises opposed tabs extending radially away from a longitudinal axis of the reducer clip.
20 The reducer clip of any of examples 16 to 19, wherein a proximal portion of the locking sleeve surrounds a portion of the proximal sleeve.
21. The reducer clip of any of examples 16 to 20, wherein the proximal sleeve further comprises a flat formed on an outer proximal portion thereof.
22. The reducer clip of any of examples 16 to 21, wherein the distal sleeve is configured to receive a proximal portion of a bone anchor within the lumen thereof.
23 An inserter-reducer shaft, comprising:
   an elongate body having a proximal portion with a flat formed thereon, a distal portion configured to rotationally drive a setscrew, a first, distal-facing shoulder formed proximal to the distal portion, and a second, proximal-facing shoulder formed proximal to the first shoulder and distal to the proximal portion;
   a sleeve disposed around the elongate body distal to the first shoulder; and
   a biasing element disposed between the sleeve and the first shoulder and configured to bias the sleeve distally relative to the elongate body.
24. The shaft of example 23, wherein the sleeve is constrained against translating relative to the elongate body beyond a distal end of the portion configured to rotationally drive a setscrew.
25. The shaft of any of examples 23 to 24, wherein the biasing element is a coil spring disposed around the elongate body between a proximal end of the sleeve and the first shoulder.
26 The shaft of any of examples 23 to 25, wherein a portion of the elongate body extending between the first shoulder and the second shoulder has an outer diameter that is less than a diameter of one or more of the first shoulder and the second shoulder.
27. The shaft of any of examples 23 to 26, wherein a portion of the elongate body extending between the first shoulder and the second shoulder has an outer diameter equal to the diameter of one or more of the first shoulder and the second shoulder.
28 The shaft of any of examples 23 to 27, wherein an outer diameter of the first shoulder and an outer diameter of the second shoulder are equal.
29 The shaft of any of examples 23 to 28, further comprising a spring clip configured to aid retention of a setscrew along the distal portion of the elongate body.
30 The shaft of any of examples 23 to 29, further comprising a thrust bearing disposed around the elongate body proximal to the second shoulder.
31. The shaft of any of examples 23 to 30, wherein the distal portion of the elongate body further comprises an insert received within a distal-facing recess of the elongate body.
32. A surgical method, comprising:
   coupling a reducer clip to a proximal end of a bone anchor;
   passing a distal portion of a shaft having a setscrew disposed thereon through a lumen of the reducer clip until a distal threaded portion of a reducer handle disposed around the shaft contacts a proximal threaded portion of the reducer clip;
   rotating the reducer handle to engage the threads of the reducer handle with the threads of the reducer clip and advance the shaft distally;
   rotating a driver handle coupled to a proximal end of the shaft extending through the reducer handle to engage threads on an outer surface of the setscrew with threads formed on an inner surface of the bone anchor.
33. The method of example 32, further comprising passing a proximal end of the shaft through a lumen of the reducer handle.
34 The method of any of examples 32 to 33, further comprising coupling the driver handle to the proximal end of the shaft extending through the reducer handle.
35. The method of any of examples 32 to 34, further comprising loading the setscrew onto a distal end of the shaft.
36. The method of example 35, wherein loading the setscrew onto the distal end of the shaft includes contacting the setscrew against a distally biased sleeve disposed around the shaft.
37. The method of any of examples 32 to 36, wherein coupling the reducer clip to the bone anchor further comprises squeezing opposed arms of the bone anchor radially inward toward one another while passing a distal end of the reducer clip over the proximal end of the bone anchor.
38. The method of example 37, wherein coupling the reducer clip to the bone anchor further comprises releasing the opposed arms of the bone anchor after passing the distal end of the reducer clip over the proximal end of the bone anchor such that opposed portions of the opposed arms of the bone anchor extend into opposed recesses formed in a sidewall of the reducer clip.
39. The method of any of examples 32 to 38, wherein coupling the reducer clip to the bone anchor further comprises proximally withdrawing a locking sleeve of the reducer clip while passing a distal portion of the reducer clip over the proximal end of the bone anchor and distally advancing the locking sleeve after the distal portion of the reducer clip is positioned over the proximal end of the bone anchor.
40. The method of any of examples 32 to 39, wherein rotating the reducer handle further comprises contacting a shoulder formed on the reducer handle against a proximal end of the reducer clip to prevent further distal advancement of the reducer handle relative to the reducer clip.
41. The method of any of examples 32 to 40, further comprising separating the reducer clip, the shaft, the reducer handle, and the driver handle as a single unit from the bone anchor after rotating the driver handle to engage the threads on the setscrew with the threads on the bone anchor.

What is claimed is:

1. A surgical system, comprising:
a reducer clip having a lumen extending from a proximal end to a distal end thereof, a recess formed in a sidewall of the reducer clip along a distal portion thereof, and threads formed in the sidewall of the reducer clip along a proximal portion thereof;
a shaft disposed through the lumen of the reducer clip;
a reducer handle having a lumen extending from a proximal end to a distal end thereof and threads formed along an outer distal portion thereof, the reducer handle threads interfacing with the threads formed along the proximal portion of the reducer clip; and
a driver handle coupled to a proximal portion of the shaft extending through the reducer handle.

1. The system of claim 1, wherein the driver handle extends proximal to the reducer handle.

2. The system of claim 1, wherein the reducer clip is a monolithic component.

3. The system of claim 1, wherein the reducer clip further comprises a locking sleeve with a locking tab at a distal end thereof that is configured to translate relative to another portion of the reducer clip.

4. The system of claim 4, wherein the reducer clip further comprises a biasing element that urges the locking sleeve distally relative to another portion of the reducer clip.

5. The system of claim 1, wherein the reducer clip further comprises opposed tabs extending radially away from a longitudinal axis of the reducer clip.

6. The system of claim 1, wherein the reducer clip further comprises a flat formed on an outer proximal portion thereof.

7. The system of claim 1, wherein the shaft further comprises a distal portion configured to rotationally drive a setscrew, a sleeve disposed around the shaft over at least part of the distal portion thereof, and a biasing element disposed proximal to the sleeve and configured to urge the sleeve distally relative to the shaft.

8. The system of claim 1, wherein the shaft further comprises a proximal-facing shoulder.

9. The system of claim 1, wherein the shaft further comprises a flat formed along a proximal portion thereof.

10. The system of claim 1, wherein the reducer handle further comprises a distal-facing shoulder disposed proximal to the threads that is configured to contact a proximal-facing portion of the reducer clip and prevent further distal advancement of the reducer handle relative to the reducer clip.

11. The system of claim 1, wherein the reducer handle further comprises a cylindrical proximal portion defining a proximal-facing recess.

12. The system of claim 1, wherein the reducer handle further comprises opposed paddles extending radially away from a longitudinal axis of the reducer handle and defining a gap therebetween at a proximal end of the reducer handle.

13. The system of claim 1, wherein the reducer clip comprises a bone anchor extension having an inner sleeve with opposed distally-extending arms and a threaded proximal portion, as well as an outer sleeve disposed over the inner sleeve and configured to translate relative to the inner sleeve to selectively lock the bone anchor extension to a bone anchor.

14. The system of claim 1, further comprising a bone anchor, wherein the reducer clip is configured to couple to a proximal portion of the bone anchor.

15. A surgical method, comprising:
coupling a reducer clip to a proximal end of a bone anchor;
passing a distal portion of a shaft having a setscrew disposed thereon through a lumen of the reducer clip until a distal threaded portion of a reducer handle disposed around the shaft contacts a proximal threaded portion of the reducer clip;
rotating the reducer handle to engage the threads of the reducer handle with the threads of the reducer clip and advance the shaft distally;
rotating a driver handle coupled to a proximal end of the shaft extending through the reducer handle to engage threads on an outer surface of the setscrew with threads formed on an inner surface of the bone anchor.

16. The method of claim 16, further comprising passing a proximal end of the shaft through a lumen of the reducer handle.

17. The method of claim 16, further comprising coupling the driver handle to the proximal end of the shaft extending through the reducer handle.

18. The method of claim 16, further comprising loading the setscrew onto a distal end of the shaft.

19. The method of claim 19, wherein loading the setscrew onto the distal end of the shaft includes contacting the setscrew against a distally biased sleeve disposed around the shaft.

20. The method of claim 16, wherein coupling the reducer clip to the bone anchor further comprises squeezing opposed arms of the bone anchor radially inward toward one another while passing a distal end of the reducer clip over the proximal end of the bone anchor.

21. The method of claim 21, wherein coupling the reducer clip to the bone anchor further comprises releasing the opposed arms of the bone anchor after passing the distal end of the reducer clip over the proximal end of the bone anchor such that opposed portions of the opposed arms of the bone anchor extend into opposed recesses formed in a sidewall of the reducer clip.

22. The method of claim 16, wherein coupling the reducer clip to the bone anchor further comprises proximally withdrawing a locking sleeve of the reducer clip while passing a distal portion of the reducer clip over the proximal end of the bone anchor and distally advancing the locking sleeve after the distal portion of the reducer clip is positioned over the proximal end of the bone anchor.

23. The method of claim 16, wherein rotating the reducer handle further comprises contacting a shoulder formed on the reducer handle against a proximal end of the reducer clip to prevent further distal advancement of the reducer handle relative to the reducer clip.

24. The method of claim 16, further comprising separating the reducer clip, the shaft, the reducer handle, and the driver handle as a single unit from the bone anchor after rotating the driver handle to engage the threads on the setscrew with the threads on the bone anchor.
